(19) United States
(12) Patent Application Publication
(10) Pub. No.: US 2026/0215726 A1
(43) Pub. Date: Jul. 30, 2026

(54) KNITTED STRAIN SENSOR SYSTEM USED FOR PHARYNGEAL REHABILITATION

(71) Applicant: Board of Regents, The University of Texas System, Austin, TX (US)

(72) Inventors: Jonathan Chen, Austin, TX (US); James Sulzer, Austin, TX (US); Corinne Jones, Austin, TX (US); David Zhang, Katy, TX (US); Drake Rowland, Austin, TX (US); Wei Li, Austin, TX (US)

(21) Appl. No.: 19/152,087

(22) PCT Filed: Feb. 1, 2024
(86) PCT No.: PCT/US2024/014008

§ 371 Date: Jul. 30, 2025

Related U.S. Application Data
(60) Provisional application No. 63/482,635, filed on Feb. 1, 2023.

(51) Int. Cl. A61B 5/00

(52) U.S. Cl. CPC A61B 5/4205; A61B 5/6804; A61B 5/6822; A61B 5/7264; A61B 2562/0261

(57) ABSTRACT

Aspects of the present invention relate to a band configured to be positioned around the neck of a subject, including a strip of knitted elastic fabric having a length and a height, one or more conductive fibers stitched or knitted onto a portion of the band and arranged into at least one knitted strain sensor, and one or more lead fibers stitched or knitted onto a portion of the band, each connected at a first end to the at least one knitted strain sensor, and at a second end to a connector.

### CROSS-REFERENCE TO RELATED APPLICATIONS

This application claims priority to U.S. Provisional Application No. 63/482,635 filed Feb. 1, 2023, incorporated herein by reference in its entirety.

### BACKGROUND OF THE INVENTION

Difficulty swallowing, known as dysphagia, affects 500,000 children and 9 million adults in the US annually [Bhattacharyya N. The prevalence of dysphagia among adults in the United States. Otolaryngology—Head and Neck Surgery. 2014; 151(5):765-9; Borowitz K C, Borowitz S M. Feeding problems in infants and children: assessment and etiology. Pediatric clinics. 2018; 65(1):59-72; Bhattacharyya N. The prevalence of pediatric voice and swallowing problems in the United States. Laryngoscope. 2015; 125:746-50]. Dysphasia can result from damage to the elaborate swallowing process that involves 40 muscles and multiple brain areas [Perlman A L, Palmer P M, McCulloch T M, Vandaele D J. Electromyographic activity from human laryngeal, pharyngeal, and submental muscles during swallowing. J Appl Physiol. 1999; 86:1663-9; Sörös P, Inamoto Y, Martin R E. Functional brain imaging of swallowing: An activation likelihood estimation meta-analysis. Hum Brain Mapp. 2009; 30:2426-39]. Dysphasia can result in aspiration, the leading cause of death in children with cerebral palsy, and even in adults with stroke and Parkinson's Disease [Troche M S, et al. Aspiration and swallowing in Parkinson disease and rehabilitation with EMST: a randomized trial. Neurology. 2010; 75(21): 1912-9; Heuschmann P U, et al. Predictors of in-hospital mortality and attributable risks of death after ischemic stroke: the German stroke registers study group. Arch Intern Med. 2004; 164(16): 1761-8]. The need to monitor dysphasia was inspired by one of the inventors' (Sulzer) five-year-old daughter who suffered a severe traumatic brain injury that has left her cognitively and motorically incapacitated with cervical dystonia that forces her head to the left [Sulzer J, Karfeld-Sulzer L S. Our child's TBI: a rehabilitation engineer's personal experience, technological approach, and lessons learned. Journal of NeuroEngineering and Rehabilitation. 2021 Dec. 7; 18(1). 10.1186/s12984-021-00862-y]. The inventor's daughter is similar to thousands of people with dysphagia who are constantly at risk of aspiration.

In rehabilitation settings, swallowing is monitored without instrumentation, with a therapist most often visually searching for movement of the larynx or using three fingers along the larynx for haptic perception of a swallow. This palpation suffers from lack of agreement with airway protection measured by x-ray video [Brates D, Molfenter S M, Thibeault S L. Assessing Hyolaryngeal Excursion: Comparing Quantitative Methods to Palpation at the Bedside and Visualization During Videofluoroscopy. Dysphagia. 2019 Jun. 15; 34(3): 298-307]. Additionally, for a caregiver such as a parent, detecting a swallow in someone with poor head control and a small neck is exceedingly difficult.

Swallowing is an intricate physiological process vital to human nutrition and health. Dysphagia, or difficulty swallowing, is a pervasive condition affecting around 1 in 25 adults annually, with approximately 9.4 million adults suffering from this issue in the United States alone [Bhattacharyya, N., The prevalence of dysphagia among adults in the United States. Otolaryngology—Head and Neck Surgery, 2014. 151(5): p. 765-769]. Swallowing disorders can arise due to a variety of causes, including neurological damage from cerebral palsy, stroke, Parkinson's disease, or traumatic brain injury (TBI) [Daniels, S. K., Neurological disorders affecting oral, pharyngeal swallowing. GI Motility online, Dysphagia can have severe consequences such as aspiration, malnutrition, dehydration, and even death, highlighting the importance of early detection and monitoring [Rofes, L., et al., Prevalence, risk factors and complications of oropharyngeal dysphagia in stroke patients: A cohort study. Neurogastroenterology & Motility, 2018. 30(8): p. e13338; Marik, P. E. and D. Kaplan, Aspiration pneumonia and dysphagia in the elderly. Chest, 2003. 124(1): p. 328-336]. The conventional approach to evaluating swallowing in clinical settings involves visual examination or manual palpation, but these methods can be inaccurate and are subject to variability between clinicians [Akai, M., Dysphagia rehabilitation manual. Japan: National Rehabilitation Center for Persons with Disabilities, 2015]. As a result, more objective, non-invasive techniques have been sought, such as videofluoroscopy and surface electromyography (sEMG), although these are expensive, time-consuming, and often inaccessible in non-clinical environments [Kenny, C., Dysphagia in Solid Malignancies outside the Head, Neck, and Upper Gastrointestinal Tract. 2019, University of Dublin].

In recent years, advances in wearable technologies have shown promise in addressing these limitations, particularly in biosensing for health monitoring. Wearable devices offer several advantages, including continuous and real-time data collection, improved patient compliance, and a greater capacity for remote monitoring and personalized care [Patel, S., et al., A review of wearable sensors and systems with application in rehabilitation. Journal of neuroengineering and rehabilitation, 2012. 9(1): p. 1-17]. Additionally, usability and adaptability are critical parameters, as devices must be affordable, comfortable, durable, easy to use, and integrate seamlessly into daily activities [Sulzer, J. and L. S. Karfeld-Sulzer, Our child's TBI: a rehabilitation engineer's personal experience, technological approach, and lessons learned. Journal of neuroengineering and rehabilitation, 2021. 18: p. 1-12].

Swallowing integrates the central nervous system and upper aerodigestive tract muscles; progressing through three distinct phases: oral, pharyngeal, and esophageal. Disruptions in these phases, or dysphagia, can derive from factors like neurological diseases and structural anomalies, posing risks like aspiration pneumonia and malnutrition and compromising quality of life [Logemann, J., Evaluation and treatment of swallowing disorders. NSSLHA Journal, 1984(12): p. 38-50]. Developing a wearable device detecting swallowing abnormalities requires extreme sensitivity to pivotal biomechanical events throughout swallowing, such as laryngeal elevation and upper esophageal sphincter (UES) opening [Pearson Jr, W. G., et al., Evaluating swallowing muscles essential for hyolaryngeal elevation by using muscle functional magnetic resonance imaging. International Journal of Radiation Oncology* Biology* Physics, 2013. 85(3): p. 735-740]. Careful consideration of sensor placement, signal-to-noise ratio, and data preprocessing is crucial to ensure accurate, reliable readings. Additionally, selecting optimal machine learning models for pattern recognition enhances the device's efficacy in distinguishing between swallowing and non-swallowing events, thereby playing a vital role in swallow classification and management across varied age groups. An in-depth comprehension of swallowing biomechanics and dysphagia-contributing factors is vital for developing a device that can adeptly classify swallowing patterns and potentially aid in early disorder management.

A novel wearable system for monitoring swallowing has the potential to help both children and adults, and any subject including animals and pets. Thus, there is a need in the art for systems and methods for monitoring swallowing. The present invention meets this need.

### SUMMARY OF THE INVENTION

Aspects of the present invention relate to a band configured to be positioned around the neck of a subject, including a strip of knitted elastic fabric having a length and a height, one or more non-conductive fibers stitched or knitted onto a portion of the band and arranged into a base structure, one or more conductive fibers stitched or knitted onto a portion of the base structure and arranged into at least one knitted strain sensor, and one or more lead fibers stitched or knitted onto a portion of the band, each connected at a first end to the at least one knitted strain sensor, and at a second end to a connector.

In some embodiments, the at least one knitted strain sensor includes a first, second and third knitted strain sensor. In some embodiments, each strain sensor is arranged horizontally along the length of the band. In some embodiments, each knitted strain sensor has a dimension along the length of the band of between 40 mm and 70 mm, and a dimension along the height of between 5 mm and 20 mm. In some embodiments, a distance between each knitted strain sensor is between 1 mm and 20 mm. In some embodiments, each knitted strain sensor is formed in a shape selected from: rectangle, oval, circle, and ellipse.

In some embodiments, the one or more non-conductive fibers are formed from a textured filament nylon yarn interwoven with nylon-covered spandex yarn, the one or more conductive fibers are formed from a coated nylon filament yarn, and the one or more lead fibers are formed from a coated nylon filament yarn. In some embodiments, the coating for the conductive fibers and lead fibers is selected from: silver, carbon nanotube, graphene, conductive materials, non-conductive materials, and combinations thereof.

In some embodiments, the system further includes adjustable fasteners positioned at each end of the length of the band configured to form the band into a loop. In some embodiments, the band is integrated into a garment.

Aspects of the present invention relate to a garment configured to be worn on a subject, including a garment having a knitted elastic fabric, one or more non-conductive fibers stitched or knitted onto a portion of the garment and arranged into a base structure, one or more conductive fibers stitched or knitted onto a portion of the base structure and arranged into at least one knitted strain sensor, and one or more lead fibers stitched or knitted onto a portion of the garment, each connected at a first end to the at least one knitted strain sensor, and at a second end to a connector.

In some embodiments, the at least one knitted strain sensor comprises a first, second and third knitted strain sensor. In some embodiments, each knitted strain sensor has a length of between 40 mm and 70 mm, and a height of between 5 mm and 20 mm. In some embodiments, a distance between each knitted strain sensor is between 1 mm and 20 mm. In some embodiments, each knitted strain sensor is formed in a shape selected from: rectangle, oval, circle, and ellipse.

In some embodiments, the one or more non-conductive fibers comprise textured filament nylon yarn interwoven with nylon-covered spandex yarn, the one or more conductive fibers comprise a coated nylon filament yarn, and the one or more lead fibers comprise a coated nylon filament yarn. In some embodiments, the coating for the conductive fibers and lead fibers is selected from: silver, carbon nanotube, graphene, conductive materials, non-conductive materials, and combinations thereof.

In some embodiments, the garment comprises a turtle-neck or sweater with a collar, and the at least one knitted strain sensor is stitched or knitted into a portion of the neck of the turtle-neck, or collar of the sweater, wherein the at least one knitted strain sensor is at least partially positioned over the laryngeal prominence of the subject.

Aspects of the present invention relate to a system for measuring swallowing performance in a subject, including a knitted strain sensor system having a band or a garment having at least one knitted strain sensor, a first computing device connected to the at least one knitted strain sensor, configured to receive signals from the at least one knitted strain sensor, and a non-transitory computer-readable medium with instructions stored thereon, which when executed by a processor, perform steps including collecting data from the at least one knitted strain sensor over a time period, calculating, from the collected data, the likelihood that a swallow was recorded during the time period, and calculating a swallow score from the collected data when the likelihood exceeds a predetermined threshold.

In some embodiments, the first computing device is positioned on the band or the garment. In some embodiments, the first computing device further comprises a wireless transceiver and the instructions further comprise collecting data from the at least one knitted strain sensor and transmitting the data via a wireless communication interface to a second computing device. In some embodiments, the instructions further comprise calculating a swallow score using a machine learning algorithm, and wherein the machine learning algorithm comprises the Chi-Squared (Chi2) algorithm.

Aspects of the present invention relate to a method of measuring swallowing performance in a subject, including: fitting a subject a knitted strain sensor system having a band or a garment with at least one knitted strain sensor, collecting strain data from the at least one knitted strain sensor over a time period, calculating a likelihood that a swallow was recorded during the time period, and calculating a swallow score from the collected data when the likelihood exceeds a predetermined threshold.

In some embodiments, the at least one knitted strain sensor comprises a first, second, and third knitted strain sensor. In some embodiments, the method further includes the step of positioning the first knitted strain sensor above the laryngeal prominence of the subject, the second knitted strain sensor on the laryngeal prominence, and the third knitted strain sensor below the laryngeal prominence. In some embodiments, the swallow score is calculated using a machine learning algorithm, and wherein the machine learning algorithm comprises the Chi-Squared (Chi2) algorithm.

In some embodiments, the method further includes the step of designating at least a portion of the time period as an event, and comparing the data collected during the portion of the time period to a set of labeled sensor data to identify the event as a swallow, a cough, breathing, a head movement, or speech. In some embodiments, the method further includes the step of transmitting the swallow score and at least a subset of the strain data to a clinician. In some embodiments, the method further includes the step of displaying strain data and swallow score from the subject to a visual display.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A and FIG. 1B depict exemplary knitted strain sensor systems according to aspects of the present invention. FIG. 1C depicts an exemplary knitted strain sensor system.

FIG. 2A and FIG. 2B depict exemplary knitted strain sensor systems according to aspects of the present invention.

FIG. 3 depicts an exemplary knitted strain sensor system according to aspects of the present invention.

FIG. 4A and FIG. 4B show two views of a subject's neck highlighting an array of numbered positions on the subject's neck including the laryngeal prominence.

FIG. 5A and FIG. 5B depict exemplary layouts for a knitted strain sensor system according to aspects of the present invention. FIG. 5A depicts an exemplary knitted strain sensor system with supporting hardware. FIG. 5B depicts an exemplary structure for a knitted strain sensor with leads and connectors.

FIG. 6 depicts a hardware diagram for an embodiment of an exemplary knitted strain sensor system according to aspects of the present invention.

FIG. 7 depicts an illustrative computer architecture for a computer for practicing the various embodiments of the invention

FIG. 8 depicts a Graphical User Interface (GUI) window for data logging of a knitted strain sensor.

FIG. 9 shows the measurement results for signal vs time for a cough, swallow and vocalization as captured by a knitted strain sensor system of the present invention.

FIG. 10 shows results from measurements as captured by a knitted strain sensor of the present invention. Graph (a) shows the results from swallowing. Graph (b) shows the results from talking. Graph (c) shows the results from coughing. Graph (d) shows the results from coughing and swallowing.

FIG. 11A-FIG. 11L show the measurement results for a knitted strain sensor system comprising a top, middle and bottom knitted strain sensor placed on a subject. FIG. 11A shows the results for a saliva swallow. FIG. 11B shows the results for swallowing 5 ml of water. FIG. 11C shows the results for swallowing 20 ml of water. FIG. 11D shows the results for swallowing a sip of water with effortful swallow. FIG. 11E shows the results for swallowing a sip of water through a straw. FIG. 11F shows the results for swallow with a head rotation to the right. FIG. 11G shows the results for a swallow with a head rotation to the left. FIG. 11H shows the results for a swallow with a chin tuck. FIG. 11I shows the results for a swallow with a cough. FIG. 11J shows the results for a swallow with an inhale and exhale. FIG. 11K shows the results for a swallow with the subject's tongue pressed against the roof of their mouth. FIG. 11L shows the results for a swallow while the subject says “ah”.

FIG. 12 depicts an exemplary method of processing swallowing data according to aspects of the present invention.

FIG. 13 depicts a comparison between a healthy and unhealthy swallow in a subject.

FIG. 14 depicts three stages of swallowing: (1) Oral Phase, (2) Pharyngeal Phase, and (3) Esophageal Phase.

FIG. 15 shows a Two-Dimensional Tensile Strain-Resistance Relationship of an exemplary knitted strain sensor for a knitted strain sensor system.

FIG. 16A, FIG. 16B, and FIG. 16C show the results for a Uniaxial Tensile Test of an exemplary knitted strain sensor system. FIG. 16A shows Resistance Trial 1, FIG. 16B shows Resistance Trial 2, and FIG. 16C shows Resistance Trial 3.

FIG. 17 shows an exemplary experimental setup and process for calibration of an exemplary knitted strain sensor system.

FIG. 18 shows an exemplary experimental setup and process for bench testing of an exemplary knitted strain sensor system.

FIG. 19 shows the results for a comparison between a 5 ml drink of water and a cough measured from a subject using a knitted strain sensor system vs sEMG data output measured from the subject.

FIG. 20 shows the results for coughing trials measured using a knitted strain sensor system comprising a bottom, middle and top sensor (in some examples referred to as first, second and third knitted strain sensors). The results indicate repeatability in an action within a subject.

FIG. 21 shows a signal comparison between unfiltered vs filtered signals measured from a subject using a knitted strain sensor system.

FIG. 22 shows the results for a coughing event measured from a subject using a knitted strain sensor system including a visualization of Standard Deviation for each curve.

FIG. 23 shows the results of a coughing event measured using a knitted strain sensor system including a visualization of Largest Dip Amplitude.

FIG. 24 shows the results of a coughing event measured using a knitted strain sensor system including a visualization of Time Duration.

FIG. 25 shows the results of a coughing event measured using a knitted strain sensor system including a visualization of Mean Crossing Rate.

FIG. 26 shows the results of a coughing event measured using a knitted strain sensor system including a visualization of Skewness and Kurtosis.

FIG. 27 shows the results of a coughing event measured using a knitted strain sensor system including a visualization of Dip and Slope.

FIG. 28 shows the results of a coughing event measured using a knitted strain sensor system including a visualization of Entropy Rate, Wavelet Entropy, Bandwidth, and Spectral Centroid.

FIG. 29 shows feature importance scores (sorted using Chi2 Algorithm) for Top Sensor (Blue), Middle Sensor (Red), Bottom Sensor (Green) for an exemplary method of measuring swallowing performance in a subject using a knitted strain sensor system according to aspects of the present invention.

FIG. 30A, FIG. 30B, and FIG. 30C show an exemplary Hyperparameter Optimization Process for an exemplary method of measuring swallowing performance in a subject using a knitted strain sensor according to aspects of the present invention. FIG. 30A shows Tuning of Model Types with exemplary presets. FIG. 30B shows Tuning of Number of Neighbors including exemplary Number of Neighbors. FIG. 30C shows Tuning of Distance Metric including an exemplary Distance Metric.

FIG. 31 shows the results for One vs Two vs Three Sensors Analysis.

FIG. 32 shows the results for gender-specific analysis.

FIG. 33A through FIG. 33F show the results for non-swallowing vs swallowing comparison. FIG. 33A shows a non-swallowing vs swallowing comparison including coughing, 20 ml water, left head rotation, and effortful swallow. FIG. 33B shows a non-swallowing vs swallowing comparison including chin tuck, 5 ml water, inhale and exhale, and sip water through straw. FIG. 33C shows a non-swallowing vs swallowing comparison including “say ah” and saliva swallow. FIG. 33D shows a non-swallowing vs swallowing comparison including coughing, 20 ml water, left head rotation, and effortful swallow. FIG. 33E shows a non-swallowing vs swallowing comparison including chin tuck, 5 ml water, inhale and exhale, and sip water through straw. FIG. 33F shows a non-swallowing vs swallowing comparison including “say ah” and saliva swallow.

FIG. 34A, FIG. 34B, and FIG. 34C show the results for measurements captured using a knitted strain sensor. FIG. 34A shows the results for a 5 ml swallow,

FIG. 34B shows the results for a 20 ml swallow, and FIG. 34C shows the results for an effortful swallow.

FIG. 35A, FIG. 35B, and FIG. 35C show images of a region of interest on a subject showing pre-swallow (FIG. 35A), mid-swallow (FIG. 35B), and post-swallow (FIG. 35C) for the subject.

FIG. 36A through FIG. 36H show the results for measurements captured using a knitted strain sensor system, as well as the analysis of the results. FIG. 36A, FIG. 36B and FIG. 36C show the results and analysis for a 5 ml swallow, 20 ml swallow and effortful swallow. FIG. 36D, FIG. 36E, show the results for KS02 5 ml swallow, KS02 effortful swallow, KS08 5 ml swallow, and KS08 effortful swallow. FIG. 36F, FIG. 36G and FIG. 36H show the results and analysis for a 5 ml swallow, 20 ml swallow, and effortful swallow.

FIG. 37 shows a comparison between video-fluoroscopy (VFSS), flexible endoscopy (FEES), surface electromyography (sEMG) microphones, and an exemplary knitted strain sensor system.

FIG. 38A through FIG. 38H show the results for measurements captured from a subject with a knitted strain sensor device vs EMG. FIG. 38A and FIG. 38B show the results for KS02 cough trial 1. FIG. 38C and FIG. 38D show the results for KS02 sip water with effortful swallow trial 1. FIG. 38E and FIG. 38F show the results for KS02 sip water through straw trial 2. FIG. 38G and FIG. 38H show the results for KS02 20 ml water trial 2.

### DETAILED DESCRIPTION

It is to be understood that the figures and descriptions of the present invention have been simplified to illustrate elements that are relevant for a clear understanding of the present invention, while eliminating, for the purpose of clarity many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human, or a human child.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Historical and real-time physiological health information is vital for the healthcare industry. Functional fabrics, once converted into garment products, open up new opportunities to sense, monitor, and measure physical signals generated by body motions and physiological processes. Some signals are dramatic (such as gait kinematics) and some are subtle (e.g. respiration, often requiring a stethoscope). Aspects of the invention provide a sensing fabric optionally integrated into a collar, capable of monitoring swallowing behavior of individuals. In certain embodiments, the invention allows for the monitoring of swallowing behavior in subjects with swallowing dysfunction known as dysphagia. Dysphagia affects 1 in 25 adults annually in the US in addition to up to 80% of children with developmental delays.

In current dysphagia clinical practice, evaluations are invasive, requiring x-ray videos or endoscopes placed through the nose. In the past 10 years, novel non-invasive measurement of swallowing has been mainly based on an accelerometer placed on the thyroid notch to identify laryngeal movement. These devices are not commercially available, are conspicuous, are uncomfortable to wear, can be difficult to mount particularly for infants and children, provide little information about specifics of swallowing physiology, and require a large thyroid cartilage for accurate measurement.

The disclosed invention is a wearable system and method for swallow measurement. The system and method provide needed and attractive advantages for both healthcare customers and healthcare providers. The disclosed fabric sensing technology overcomes the disadvantages found in current dysphagia clinical practice and provides users with a comfortable garment for sensitive, unobtrusive swallow measurement and monitoring.

### Knitted Strain Sensor System

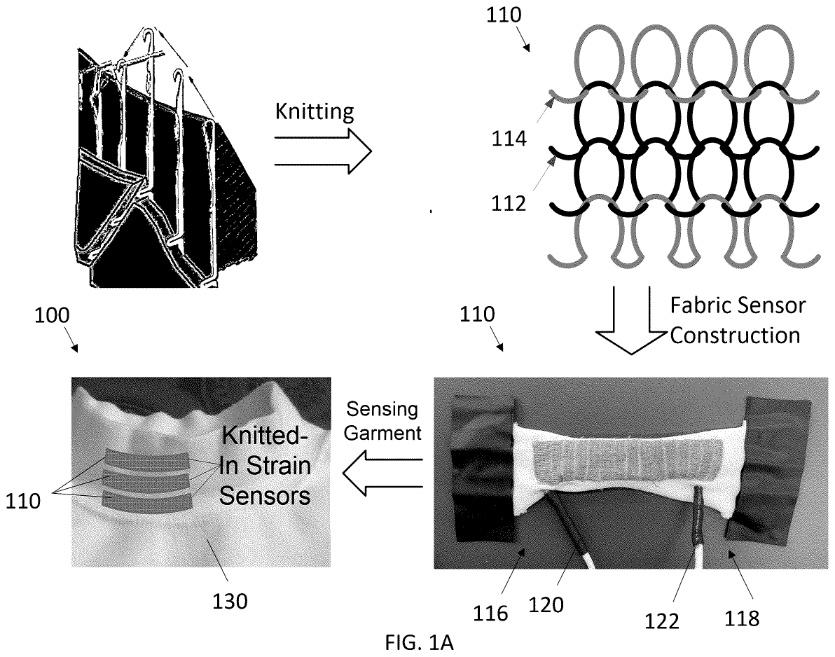

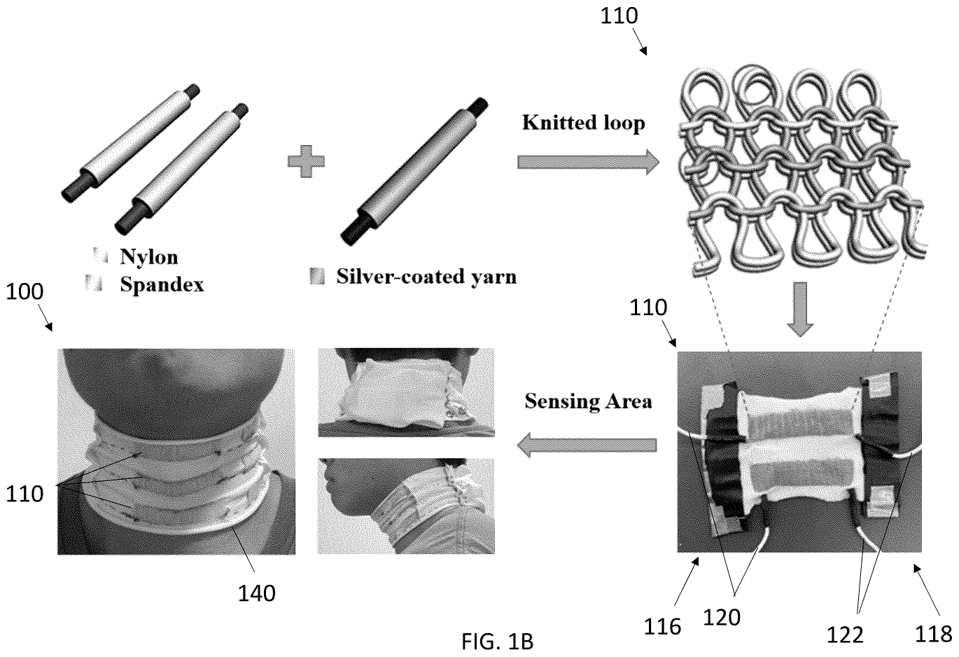

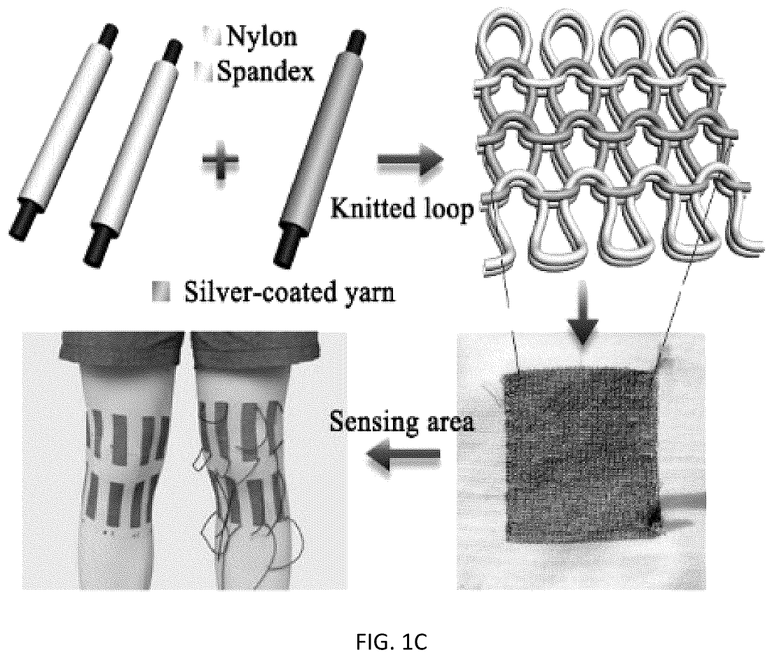

The disclosed knitted strain sensor system provides a comfortable fabric-based swallowing sensor usable on all subjects but especially on human adults and children. As contemplated herein, a knitted strain sensor system is now described. Referring now to FIG. 1A and FIG. 1B, shown are exemplary knitted strain sensor system 100 and fabrication methods thereof according to various aspects of the present invention. In some embodiments, system 100 comprises at least one knitted strain sensor 110 (in some examples referred to as a fabric sensor, knitted strain sensor, or sensing fabric) fixedly attached to, woven into, and/or embedded within, a garment 130 or band 140. Knitted strain sensor 110 comprises at least one structure of fibers comprising at least a first fiber 112 interwoven with a second fiber 114 forming at least one sensing area or region. In some embodiments, system 100 further comprises at least one conductive fiber forming one or more electrical leads connected to the at least one knitted strain sensor 110. In some embodiments, system 100 further comprises a computer 700 electronically connected to the at least one knitted strain sensor 110.

Knitted strain sensor 110 comprises a series of interconnecting loops formed by a conductive fiber or yarn that form a sensing area or region having at least a first end 116 and a second end 118. Without any external load, knitted strain sensor 110 is under a total relaxation condition to indicate its original lowest electrical resistance (R₀). Under a tensile deformation, knitted strain sensor 110 resistance increases in response to the tensile strain increase, because of a decrease in inter-loop contact area or force.

Knitted strain sensor 110 may comprise a separate fiber structure from garment 130 or band 140, or may comprise one or more fibers interwoven into an existing garment or band. In some embodiments, knitted strain sensor 110 comprises a coated conductive fiber applied to a subset of band 140 (e.g, a knitted elastic fabric), the subset arranged into at least one knitted strain sensor 110. In some embodiments, knitted strain sensor 110 comprises at least one coated conductive fiber applied or interwoven into to at least a portion of band 140 or garment 130. In some embodiments, a coated conductive fiber is arranged into at pattern in band 140 or garment 130 thereby forming a pattern or plurality of knitted strain sensor 110, in a continuous series of knitted strain sensors or connected in various configurations (parallel and/or serial).

Although certain embodiments are presented herein having interwoven fibers including a certain pattern, for example the weft knitting of FIG. 1A and FIG. 1B, it is understood that sensors as contemplated herein could be knitted using any suitable knitting stitch, including but not limited to a garter stitch, a stockinette stitch, a double stockinette stitch, a rib stitch, a seed stitch, a moss stitch, a brioche stitch, a broken rib stitch, or any other suitable stitching pattern or combination of these.

In some embodiment, knitted strain sensor 110 is positioned along a portion of band 140 and/or garment 130 wherein the at least one sensing area or region of the at least one knitted strain sensor 110 is positioned near or touching an area or region of interest on a subject. In some embodiments, the at least one knitted strain sensor 110 is positioned or configured in one or more positions or patterns on band 140 and/or garment 130. In some embodiments, the at least one knitted strain sensor 110 is arranged vertically along the height of band 140 and centered at an axis on the length. In some embodiments, at least one knitted strain sensor 110 comprises a first, second, and third knitted strain sensor arranged or patterned along at least a portion of garment 130 or band 140. In some embodiments, the knitted strain sensors are positioned in upper, middle, and lower positions on garment 130 or band 140. In some embodiments, system 100 comprises a plurality of knitted strain sensor 110. In some embodiments, the positions of the at least one knitted strain sensor 110 on band 140 and/or garment 130 are positioned such that the garment or band is positioned around the subject's neck, tightly covering the major area around the laryngeal prominence.

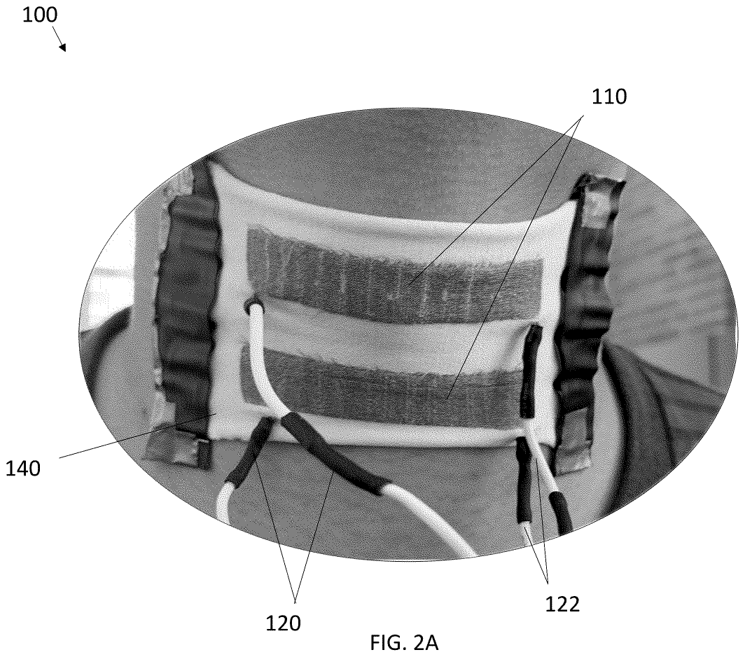

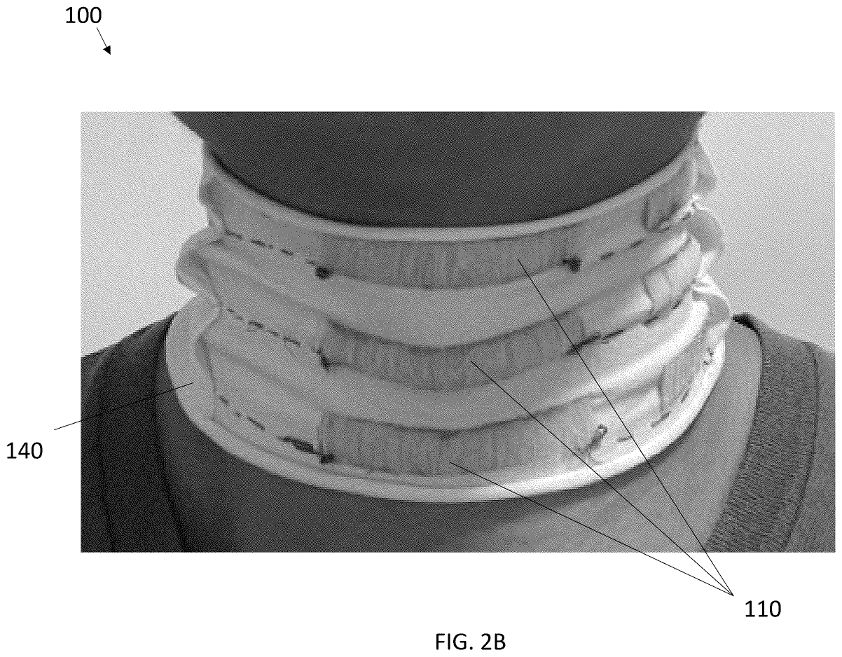

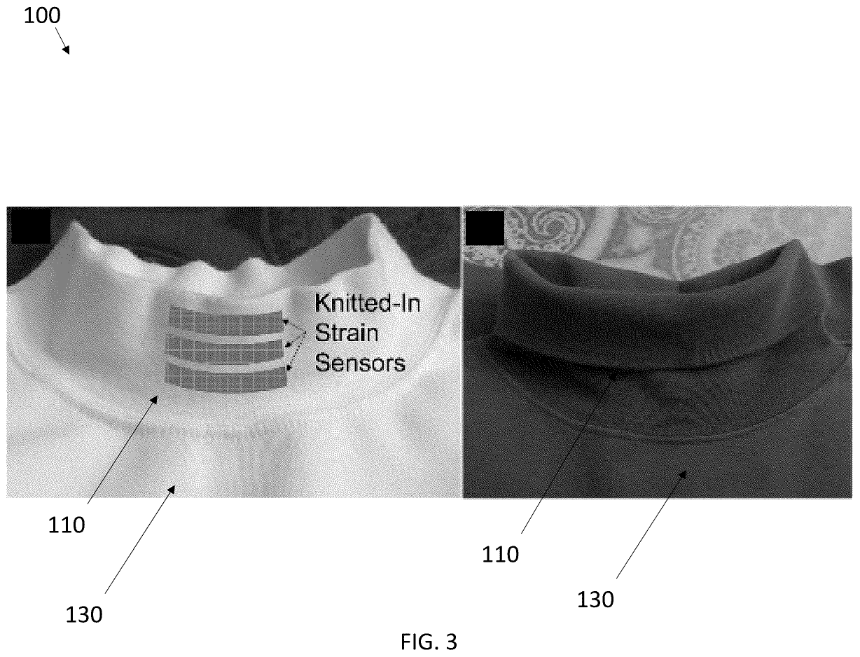

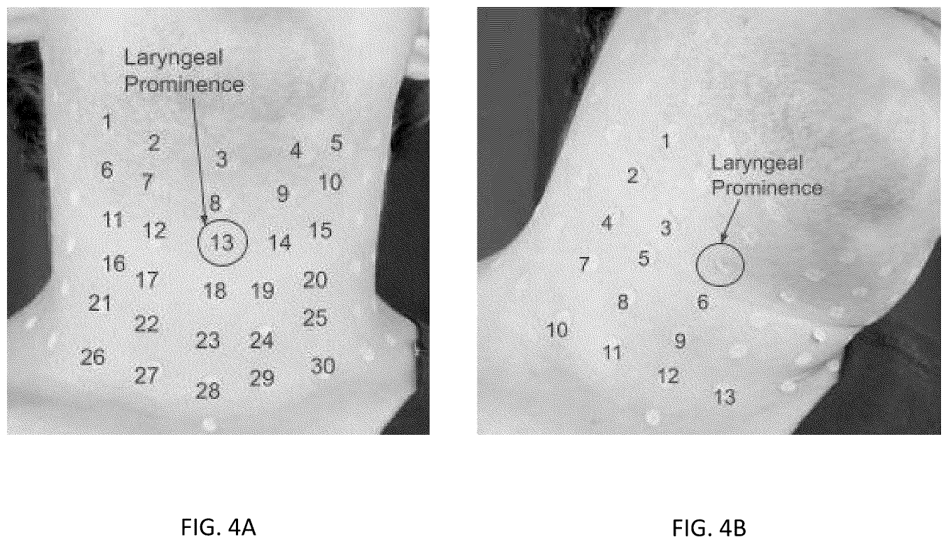

Referring now to FIG. 2A and FIG. 2B, system 100 is depicted having a knitted structure with a stable and stretchy fabric base (i.e., band 140) and a strain sensing area (i.e., knitted strain sensor 110). In some embodiments, system 100 is configured to be positioned around the neck of a subject (FIG. 2B). In some embodiments, system 100 can be produced as a medical collar or sewn into a t-shirt as a comfortable garment (FIG. 3).

In some embodiments, each knitted strain sensor 110 comprises at least one terminal, connector or electrical lead for connecting to a lead wire, another knitted strain sensor 110, circuit, and/or computing device (e.g., computer 700, laptop, tablet, desktop, and the like). In some embodiments, system 100 comprises at least one conductive knitted electrical lead connected to the at least one knitted strain sensor 110. In some embodiments, the knitted electrical lead is embedded or woven into garment 130 or band 140. In some embodiments, the electrical lead terminates with one or more terminals. In some embodiments, the one or more terminals comprise one or more connectors. It should be appreciated that any connector, including electrical connectors, may be used. In some embodiments, each knitted strain sensor 110 has a first and a second terminal. In some embodiments, each terminal may comprise a button, post, pole, contact, connector lead, or the like. In some embodiments, each terminal is a metal button for connecting a lead. In some embodiments, a first electrical lead 120 electrically and fixedly attaches to a position on first end 116 of knitted strain sensor 110, and a second electrical lead 122 attaches to a position on second end 118.

Aspects of the present invention relate to various materials and coatings for each fiber of the at least one knitted strain sensor 110. Each fiber may be formed from or comprise any suitable material as would be known by one of ordinary level of skill in the art, including, but not limited to, cotton, silk, linen, wool, polyester, nylon, rayon, spandex, conductive polymers, metal, metal alloys, super alloy, conductive materials, superconductive materials and combinations thereof.

Portions or all of system 100 may comprise any coating, including but not limited to conductive or non-conductive coatings, known by one of ordinary level of skill int the art. For example, in some embodiments, each fiber of knitted strain sensor 110 (e.g., first fiber 112, second fiber 114) comprises at least one coating on the fiber. The at least one coating may comprise any suitable coating as would be known by one of ordinary level of skill in the art, including, but not limited to, silver, copper, carbon nanotube, graphene, conductive materials, and any combinations thereof.

In some embodiments, each fiber comprises at least one knitted yarn and/or fabric. In some embodiments, the at least one fiber 112 comprises a textured nylon filament yarn (75 dtex, 36f) and/or a nylon-covered spandex yarn (50 dtex), and fiber 114 comprises a silver-coated nylon filament yarn (40 dtex, 12f) and/or nylon-covered spandex yarn. In some embodiments, each fiber comprises a core and a sheath, wherein the core may comprise a first fiber material, and the sheath comprises a second fiber material. In some embodiments, each of the core and sheath may comprise one or more coatings as contemplated herein. In some embodiments, the sheath discontinuously abuts the core along the length of each fiber.

In some embodiments, the fibers of knitted strain sensor 110 may comprise at least one conductive fiber. In some embodiments, the conductive fiber of knitted strain sensor 110 has at least one coating. In some embodiments, knitted strain sensor comprises a weft-knitting of a nylon/nylon-wrapped spandex/silver coated yarn. It should be appreciated that in various embodiments, first fiber 112 and/or second fiber 114 may comprise any conductive or non-conductive fiber, yarn or material known in the art to form the required sensing areas or regions of knitted strain sensor 110.

In some embodiments, garment 130 and/or band 140 may be formed or manufactured from any fiber, yarn, fabric or material known in the art. For example, in some embodiments, the at least one band 140 is formed of a strip of fabric, or a strip of knitted elastic fabric. It should be appreciated that knitted strain sensor 110 may be interwoven into existing garments (e.g., garment 130) or bands (e.g., band 140), or may form portions of, or the entire garment or band to form a system 100. In some embodiments, garment 130 is a sweater, a turtle-neck, a collared shirt, a jacket, a neck gaiter, a head sock, an arm-band, a leg-band, or the like.

In some embodiments, band 140 comprises at least one fastener positioned at each end of the length of band and configured to form band 140 into a loop. In other embodiments, band 140 comprises first and second ends, with adjustable fasteners positioned on the first and second ends configured to affix band 140 to the subject.

In some embodiments, band 140 is formed at least partially in the shape of a rectangle, square, or circle. In some embodiments, band 140 has an oval shape. In some embodiments, band 140 has a dimension along the length of between 100 mm and 1000 mm, and a dimension along the height of between 30 mm and 500 mm.

Each knitted strain sensor is at least partially formed in one or more shapes. For example, in some embodiments, each knitted strain sensor 110 has a rectangular shape, a circular shape, or an oval shape. In some embodiments, each knitted strain sensor 110 has a dimension along the length of between 40 mm and 100 mm, and a dimension along the height of between 5 mm and 20 mm. In some embodiments, each knitted strain sensor 110 has a diameter ranging from 50 mm to 250 mm. In some embodiments, each knitted strain sensor 110 has a circumference ranging from 250 mm to 600 mm. In some embodiments, a distance between each knitted strain sensor 110 in the at least one knitted strain sensor 110 or plurality of knitted strain sensor 110 is between 1 mm and 20 mm.

In some embodiments, an exemplary fabrication method comprises knitting a knitted strain sensor 110 comprising at least a first fiber 112 interwoven with second fiber 114, attaching electrical leads to knitted strain sensor 110, and attaching knitted strain sensor 110 to at least one garment 130 forming an exemplary system 100 comprising a wearable garment.

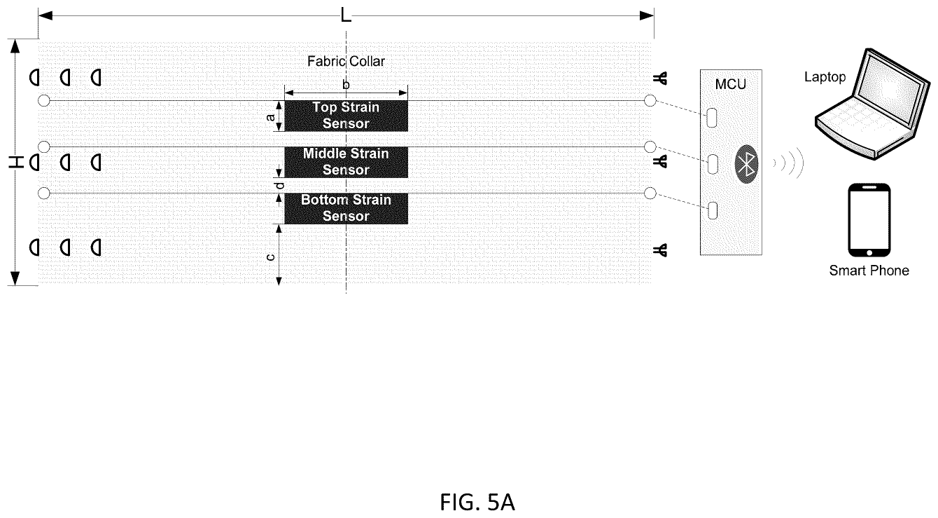

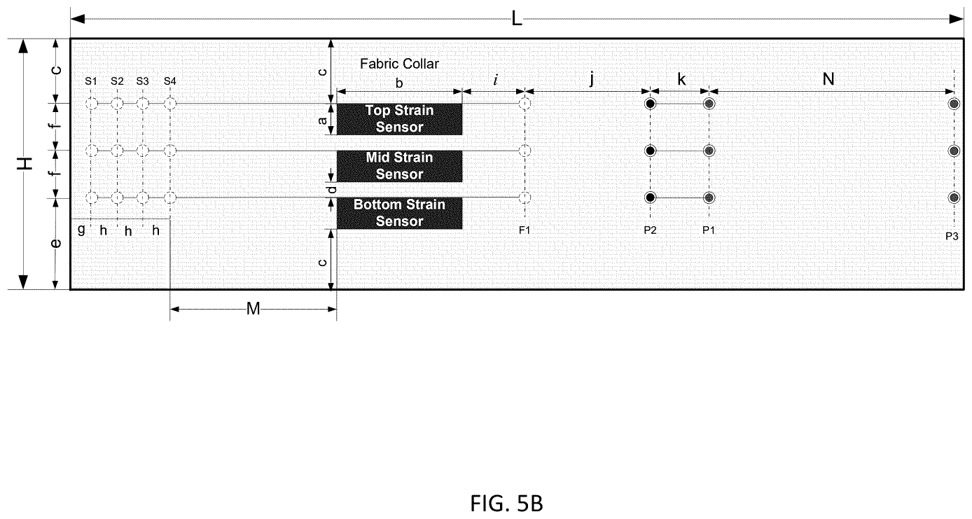

In alternative embodiments, system 100 comprises a filling-knitted fabric with two structures: base structure and sensing structure. In some embodiments, the base structure comprises a textured nylon filament yarn (75 dtex, 36f) and a nylon-covered spandex yarn (50 dtex). In some embodiments, the sensing structure comprises a silver-coated nylon filament yarn (40 dtex, 12f) and the same nylon-covered spandex yarn. In some embodiments, the fabric comprises a single sensing area (single sensor) or multiple sensing areas (multiple sensors). In some embodiments, for the swallowing measurement, three sensor areas are designed, each having two leads for hardware connection. In some embodiments, these leads are the same conductive yarn used in the sensing area but knitted in the fabric base structure as a long single course of loops. In some embodiments, metal snap buttons are attached onto each end of the leads as connecting points for the MCU unit (FIG. 5B). In some embodiments, the sensing fabric is produced using a single circular knitting machine with 28E machine gauge.

Aspects of the present invention relate to qualities and usability of system 100 according to aspects of the present invention. Usability is a broad concept, composed of affordability, comfort, robustness, ease of use and especially the ability to integrate the device into daily activities [Sulzer J, Karfeld-Sulzer L S. Our child's TBI: a rehabilitation engineer's personal experience, technological approach, and lessons learned. Journal of NeuroEngineering and Rehabilitation. 2021 Dec. 7; 18(1). 10.1186/s12984-021-00862-y]. In some embodiments, system 100 is affordable, comfortable to wear, has a robust design, and is easy to use. In some embodiments, system 100 is hypoallergenic. In some embodiments, system 100 is non-invasive, stable, tough, and secure. In some embodiments, system 100 has fabric that is soft, stretchy and/or breathable. In some embodiments, system 100 is comfortable to wear and thus tolerable to children. In some embodiments, system 100 is sufficiently low profile to be able to be integrated into existing clothing and thus into activities of daily living. In some embodiments, there is no need to carry the system or retrieve it for daily use. In some embodiments, system 100 is robust to aberrant head movement common in children with cerebral palsy.

In some embodiments, system 100 is applied to measure and monitor swallowing behavior in a subject. For example, in certain instances, system 100 is applied to measure and monitor the swallowing behavior in pediatric subjects, which is critical but very difficult to implement in current clinical practice. In some embodiments, swallowing signals from system 100 are collected by a data acquisition unit and subsequently displayed on a computing device (e.g., computer 700, laptop, tablet, desktop, and the like). In some embodiments, system 100 further comprises a swallow measurement device integrated with the knitted strain sensor and wearable MCU unit, and networked with application software (FIG. 5A).

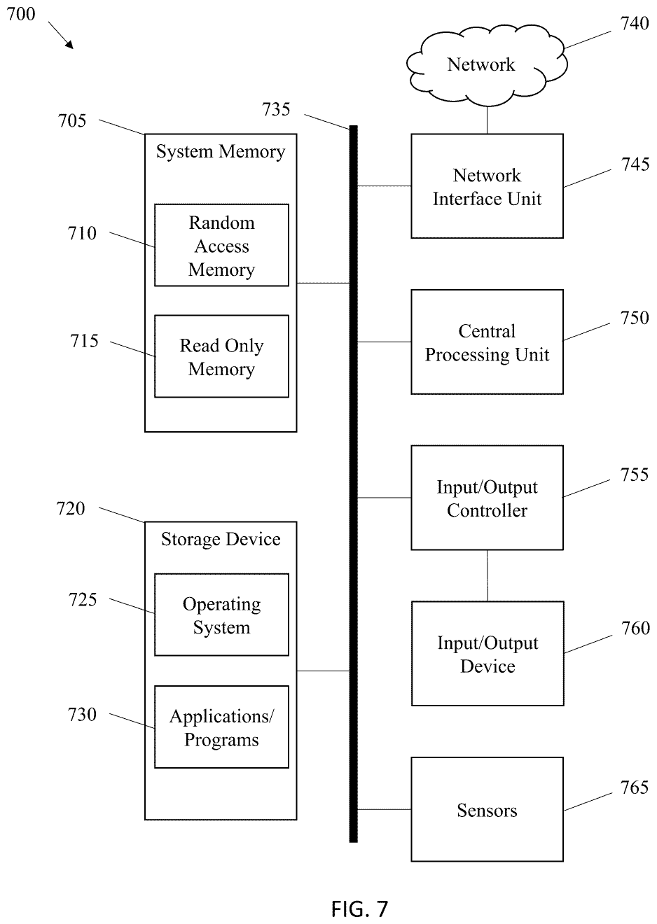

In some embodiments, system 100 comprises at least one computing device. In some embodiments, the at least one computing device is computer 700 as elaborated upon below and shown in FIG. 7. In some embodiments, system 100 comprises a non-transitory computer-readable medium with instructions stored thereon. In some embodiments, the plurality of knitted strain sensor 110 are connected to the computing device. In some embodiments, the computing device is configured to receive signals from the plurality of knitted strain sensor 110. In some embodiments, the first computing device is positioned on the band (e.g., band 140).

In some embodiments, system 100 comprises a first computing device connected to the plurality of knitted strain sensor 110. In some embodiments, the first computing sensor is configured to receive signals from the knitted strain sensor 110. In some embodiments, system 100 further comprises a non-transitory computer-readable medium with instructions stored thereon, which when executed by a processor, perform steps comprising collecting data from the knitted strain sensors over a time period, calculating, from the collected data, the likelihood that a swallow was recorded during the time period, and calculating a swallow score from the collected data when the likelihood exceeds a predetermined threshold.

In some aspects, the present invention relates to a system 100 comprising a Bluetooth-ready mini MCU that can be attached to the knitted strain sensor to form a complete wearable system. In some embodiments, system 100 will perform collection of the knitted strain sensor signals (e.g., signals from at least one knitted strain sensor 110), A/D conversion, and data transmission through an embedded Bluetooth module. In some embodiments, system 100 will involve a low power Bluetooth LE module that minimizes power consumption and can handle the small size of data (~1 Kb/s). In some embodiments, system 100 comprises a nRF51822 system-on-a-chip (Nordic Semiconductor) connected to a microcontroller (Arduino Pro Mini), and powered by a 400-hr battery. In some embodiments, data from system 100 will be acquired at 40 Hz. In some embodiments, system 100 connects to an Android smartphone via a wireless communication protocol, for example Bluetooth Low Energy (BLE) or Bluetooth, or any other wireless communication protocol. In some embodiments, the microcontroller will send the raw sensing data to the Android device or laptop receiver where the data will be processed. In some embodiments, system 100 will be smaller than a quarter, be unobtrusively worn in a shirt collar, and/or be configured to be easily removed for washing. In some embodiments, system 100 will be waterproof.

In some embodiments, the signal from system 100 will be processed at the receiver end starting with a three-point running average filter with a five-second sliding window for analysis. In some embodiments, data from system 100 will be processed in a machine learning classifier as described below.

### Computing Device

Aspects of the present invention relate to the system having at least one computing device. In some embodiments, the system comprises a software executing instructions provided may be stored on a non-transitory computer-readable medium, wherein the software performs some or all of the steps of the present invention when executed on a processor.

In some embodiments, the first computing device further comprises a wireless transceiver and the instructions further comprise collecting data from the sensors and transmit the data via a wireless communication interface to a second computing device. In some embodiments, the instructions further comprise calculating the swallow score using a machine learning algorithm. The algorithm can reside in the second computing device.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

FIG. 7 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 7 depicts an illustrative computer architecture for a computer 700 for practicing the various embodiments of the invention. The computer architecture shown in FIG. 7 illustrates a conventional personal computer, including a central processing unit 750 (“CPU”), a system memory 705, including a random access memory 710 (“RAM”) and a read-only memory (“ROM”) 715, and a system bus 735 that couples the system memory 705 to the CPU 750. A basic input/output system containing the basic routines that help to transfer information between elements within the computer, such as during startup, is stored in the ROM 715. The computer 700 further includes a storage device 720 for storing an operating system 725, application/program 730, and data.

The storage device 720 is connected to the CPU 750 through a storage controller (not shown) connected to the bus 735. The storage device 720 and its associated computer-readable media provide non-volatile storage for the computer 700. Although the description of computer-readable media contained herein refers to a storage device, such as a hard disk or CD-ROM drive, it should be appreciated by those skilled in the art that computer-readable media can be any available media that can be accessed by the computer 700.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 700 may operate in a networked environment using logical connections to remote computers through a network 740, such as TCP/IP network such as the Internet or an intranet. The computer 700 may connect to the network 740 through a network interface unit 745 connected to the bus 735. It should be appreciated that the network interface unit 745 may also be utilized to connect to other types of networks and remote computer systems.

The computer 700 may also include an input/output controller 755 for receiving and processing input from a number of input/output devices 760, including a keyboard, a mouse, a touchscreen, a camera, a microphone, a controller, a joystick, or other type of input device. Similarly, the input/output controller 755 may provide output to a display screen, a printer, a speaker, or other type of output device. The computer 700 can connect to the input/output device 760 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 720 and/or RAM 710 of the computer 700, including an operating system 725 suitable for controlling the operation of a networked computer. The storage device 720 and RAM 710 may also store one or more applications/programs 730. In particular, the storage device 720 and RAM 710 may store an application/program 730 for providing a variety of functionalities to a user. For instance, the application/program 730 may comprise many types of programs such as a word processing application, a spreadsheet application, a desktop publishing application, a database application, a gaming application, internet browsing application, electronic mail application, messaging application, and the like. According to an embodiment of the present invention, the application/program 730 comprises a multiple functionality software application for providing word processing functionality, slide presentation functionality, spreadsheet functionality, database functionality and the like.

The computer 700 in some embodiments can include a variety of sensors 765 for monitoring the environment surrounding and the environment internal to the computer 700. These sensors 765 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Aspects of the invention relate to machine learning executed on a computing device, wherein the computing device may resemble computer 700. Machine learning is a type of artificial intelligence (AI) that provides systems the ability to learn and improve from experience without being explicitly programmed. Machine learning utilizes algorithms to analyze data sets and identify correlations and patterns, and then uses those patterns to make predictions and decisions. In general, machine learning models fall into three primary categories: supervised machine learning, unsupervised machine learning and semi-supervised machine learning.

Supervised learning, is defined by its use of labeled datasets to train algorithms to classify data or predict outcomes accurately. As input data is fed into the model, the model adjusts its weights until it has been fitted appropriately. Some methods used in supervised learning include neural networks, naïve bayes, linear regression, logistic regression, random forest, and support vector machine (SVM).

Unsupervised learning, uses machine learning algorithms to analyze and cluster unlabeled datasets. These algorithms discover hidden patterns or data groupings without the need for human intervention. Principal component analysis (PCA) and singular value decomposition (SVD) are two common approaches for this. Other algorithms used in unsupervised learning include neural networks, k-means clustering, and probabilistic clustering methods.

Semi-supervised learning offers a medium ground between supervised and unsupervised learning. During training, semi-supervised learning uses a smaller labeled data set to guide classification and feature extraction from a larger, unlabeled data set.

Classification is a part of supervised learning (learning with labeled data) through which data inputs can be easily separated into categories. In machine learning, there can be binary classifiers with only two outcomes (e.g., spam, non-spam) or multi-class classifiers (e.g., types of books, animal species, etc.). A popular classification algorithm is a decision tree whereby repeated questions leading to precise classifications can build an “if-then” framework for narrowing down the pool of possibilities over time.

Clustering is a form of unsupervised learning (learning with unlabeled data) that involves grouping data points according to features and attributes. The most common kind of clustering is K-means clustering, which involves representing each cluster by a variable “k” and then defining the centroid of those clusters.

Regression is a type of structured machine learning algorithm where inputs and outputs can be labelled. Linear regression provides outputs with continuous variables (any value within a range), such as pricing data. Logistical regression is when variables are categorically dependent and the labeled variables are precisely defined. For example, you can classify whether a store is open as (1) or (0), but there are only two possibilities.

Deep learning is an application of machine learning that imitates the workings of the human brain. Deep learning networks interpret big data, both unstructured and structured, and recognize patterns. Neural networks are closely related to deep learning, they create sequential layers of neurons that deepen the understanding of data collected from a machine to provide an accurate analysis. A neural network consists of layers of nodes, having neurons, which receive stimulation from “trigger” data. This data then is assigned a weight through coefficients, as some data inputs may be more significant than others. Neurons normally come in three different layers: an input layer of data, a hidden layer with mathematical computations, and an output layer.

The Chi-Squared (Chi2) algorithm works by comparing observed data with expected data to assess how well they align. In the context of feature selection, it evaluates the independence between each feature and the class labels. The underlying principle is that features should be closely related to the class label and relatively independent from each other.

Mathematically, for each feature, the Chi2 statistic is calculated using the formula:

$${X}^{2} = \sum \frac{{( {O}_{ij} - {E}_{ij} )}^{2}}{{E}_{ij}} \qquad Equation 1$$

where Oᵢⱼ represents the observed frequency and Eᵢⱼ represents the expected frequency under the assumption of independence. A high Chi2 score implies that the feature and the class label are dependent, and thus the feature is important for classification. Conversely, a low Chi2 score indicates that the feature is likely not important, as its distribution does not depend on the class label.

The Chi2 algorithm was used to determine the independence between the extracted features from knitted strain sensor data and the class labels, which were whether the subject was swallowing or not. The features that showed the most dependency (and hence, had the highest Chi2 scores) were deemed to be the most significant in differentiating between swallowing and non-swallowing actions.

These top-ranked features for each sensor, assigned an importance score based on their Chi2 statistic, were then presented and analyzed in terms of their relevance to the mechanics of the swallowing action. This application of the Chi2 algorithm not only aids in understanding the relationship between the features and the swallowing process, but also serves as an effective feature selection method for machine learning tasks, particularly for the included dataset from healthy participants.

### Methods of Use

Aspects of the present invention relate to exemplary methods of use for any disclosed knitted strain sensor system. Various disclosed methods comprise one or more feature extractions from the data measured from the knitted strain sensors. In summary, the feature extraction methods disclosed herein aim to capture the essential characteristics of swallowing and non-swallowing actions through a combination of time-domain, frequency-domain, and statistical features. These features were selected based on their relevance to the physiological processes involved in swallowing and their potential to differentiate between swallowing and non-swallowing patterns. By analyzing the extracted features, healthcare professionals can gain valuable insights into the swallowing process, ultimately leading to improved assessment and management of dysphagia in patients.

Aspects of the present invention relate to a method of measuring swallowing performance in a subject. In some embodiments, the method comprises fitting a subject a knitted strain sensor system having a band or a garment with at least one knitted strain sensor, collecting strain data from the at least one knitted strain sensor over a time period, calculating a likelihood that a swallow was recorded during the time period, and calculating a swallow score from the collected data when the likelihood exceeds a predetermined threshold. It should be appreciated that the subject may be fitted with the fabric sensor system on various parts of the body, including, but not limited to, around the neck, in contact with the neck, surrounding at least a portion of the neck, over a least a portion of the laryngeal prominence, above, over and below the laryngeal prominence, over at least a portion of the larynx, over the throat, or any combination thereof. For example, in some embodiments, the middle sensor is placed directly on the larynx, the top sensor above the larynx but below the chin, and the bottom sensor below the larynx to capture head and body movements (FIG. 2B). Or in another example, the first knitted strain sensor is positioned above the laryngeal prominence of the subject, the second knitted strain sensor on the laryngeal prominence, and the third knitted strain sensor below the laryngeal prominence. Or in another example, a superior sensor: 1 cm above thyroid notch; middle sensor: at thyroid notch; inferior sensor: 1 cm below thyroid notch.

In some embodiments, the method further comprises the step of positioning the first knitted strain sensor above the laryngeal prominence of the subject, the second knitted strain sensor on the laryngeal prominence, and the third knitted strain sensor below the laryngeal prominence. In some embodiments, the swallow score is calculated using a machine learning algorithm, and wherein the machine learning algorithm comprises the Chi-Squared (Chi2) algorithm.

In some embodiments, the method further comprises the step of designating at least a portion of the time period as an event, and comparing the data collected during the portion of the time period to a set of labeled sensor data to identify the event as a swallow, a cough, breathing, a head movement, or speech. In some embodiments, the method further comprises the step of transmitting the swallow score and at least a subset of the strain data to a clinician. In some embodiments, the method further comprises the step of displaying strain data and swallow score from the subject to a visual display.

It should be appreciated that the disclosed systems and methods may be used on any subject or patient, including but not limited to, human subjects, non-human subjects, dogs, cats, horses, and other vertebrates. For example, but without limitation, the subject may be a human infant, a human child, a human adolescent, or a human adult.

In some aspects, the present invention relates to a method of measuring swallowing performance in a subject, comprising the steps of fitting a subject with a band having a plurality of knitted strain sensors, collecting strain data from the plurality of knitted strain sensors over a time period, calculating a likelihood that a swallow was recorded during the time period, and calculating a swallow score from the collected data when the likelihood exceeds a predetermined threshold. In some embodiments, the plurality of knitted strain sensors comprises three knitted strain sensors. In some embodiments, the swallow score is calculated using a machine learning algorithm. In some embodiments, the machine learning algorithm is the Chi-Squared (Chi2) algorithm.

In some embodiments, the method further comprises the steps of designating at least a portion of the time period as an event, and comparing the data collected during the portion of the time period to a set of labeled sensor data to identify the event as a swallow, a cough, breathing, a head movement, or speech. In some embodiments, the method further comprises the step of transmitting the swallow score and at least a subset of the strain data to a clinician. In some embodiments, the method further comprises the step of displaying real-time signals (i.e. strain data) and swallow score from the subject to a visual display.

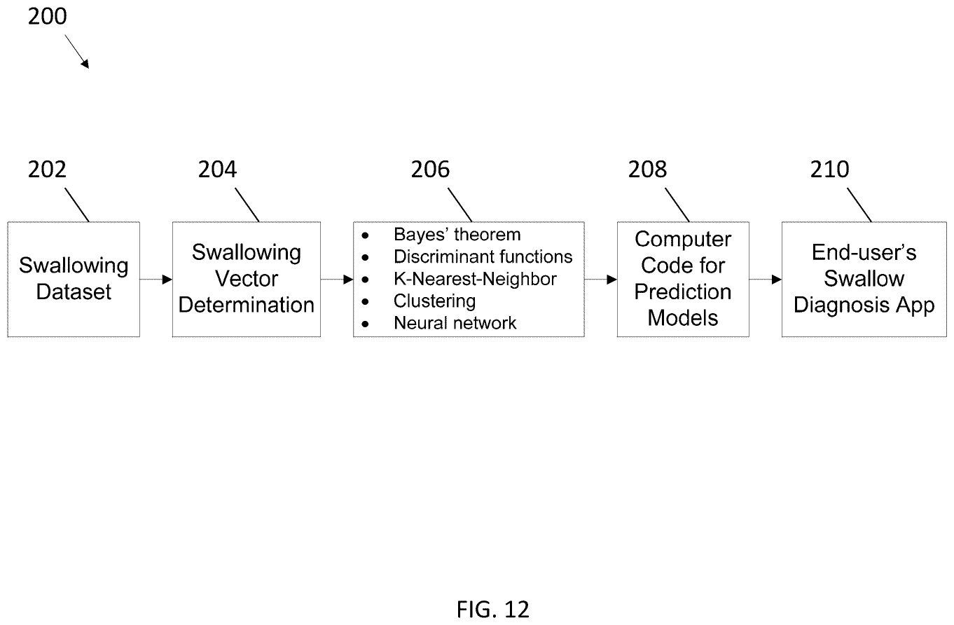

Aspects of the present invention relate to a method for processing swallowing data using machine learning. Referring now to FIG. 12, an exemplary method 200 for processing swallowing data is shown. In some embodiments, method 200 comprises the steps of: 202 recording a swallowing dataset, 204 determining vectors for the swallowing data, 206 employing machine learning processes, including, but not limited to: using Bayes' theorem, using discriminant functions, using K-nearest-neighbor, using clustering, and using a neural network, 208 providing a computer code for prediction models, and 210 displaying the processed swallowing data and/or swallow diagnosis in a UI.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Knitted Strain Sensor System

The disclosed system provides a comfortable fabric-based swallowing sensor usable in any subject, but is especially useful for children. This sensor was initially developed for the purpose of detecting human movement [Li Y, Miao X, Chen J Y, Jiang G, Liu Q. Sensing performance of knitted strain sensor on two-dimensional and three-dimensional surfaces. Materials & Design. 2021 January; 197. 10.1016/j.matdes.2020.109273]. It utilized weft-knitting of a nylon/nylon-wrapped spandex/silver coated yarn to form required sensing areas. The study indicated that the fabric strain sensor exhibited good sensing performance under both 2D and 3D fabric surface strains with corresponding time 400 ms and 350 ms respectively. Strain sensing sensitivity could reach 60% for a uniaxial tension in the 2D fabric surface, and 120% for all-direction tension in the 3D fabric surface (FIG. 1C). The disclosed system is sufficiently low profile to be able to be integrated into existing clothing and thus into activities of daily living. There is no need to remember to carry the device or retrieve it for daily use. The sensor is robust to aberrant head movement common in children with cerebral palsy. It is able to communicate wirelessly through smartphone apps. Most importantly, the disclosed device is comfortable to wear and thus tolerable to children.

The ultimate product from this project is a knitted swallow measurement device integrated with the proved fabric strain sensor and wearable MCU unit, and networked with application software. It can be produced as a medical collar or sewn into a t-shirt as a comfortable garment (FIG. 3). The value proposition of this transformative technology is that the fabric strain sensor device provides a non-invasive and comfortable testing tool for dysphagia patients of all ages, enabling a significant increase in device users and cost reduction for both healthcare customers and providers.

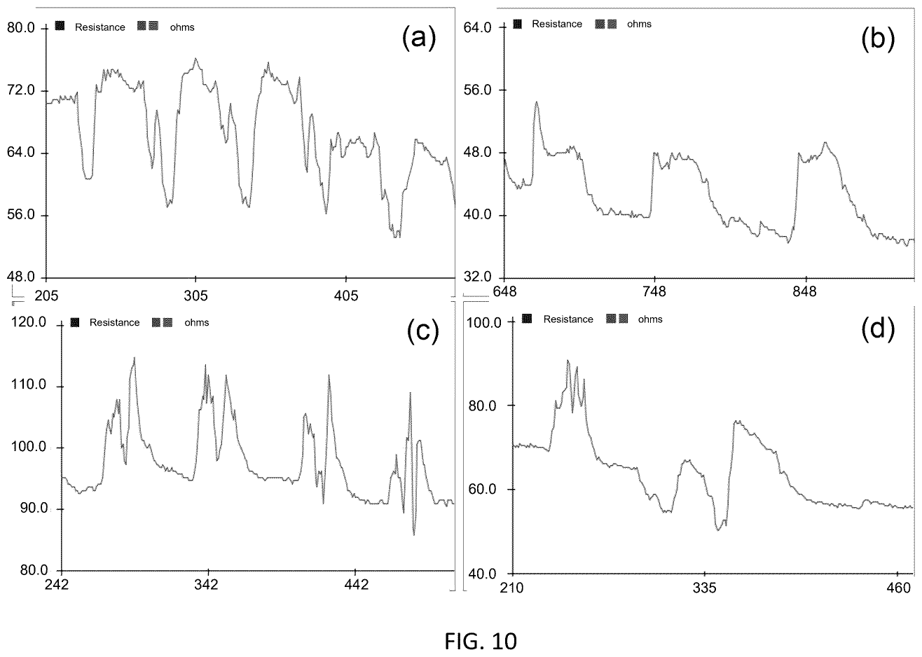

The creation of this fabric sensing technology includes the strain sensor fabric, mechatronic signal acquisition, testing procedure, and data analysis for parameter extraction and swallow decoding. The sensing fabric is made from a knitted structure composed of a stable and stretchy fabric base and a strain sensing area. The developed fabric strain sensor technology is applied to measure and monitor swallowing behavior that is critical to pediatric patients but very difficult to implement in current clinical practice. The fabric is draped around the subject's neck, tightly covering the major area around the laryngeal prominence. Swallowing signals are collected by a data acquisition unit and subsequently displayed on a laptop. The disclosed experimental data reveals that the fabric strain sensor can identify swallowing consistently in comparison to vocalization and coughing (FIG. 10).

Sensing Design and Fabrication: the fabric strain sensor is made of a filling-knitted fabric with two structures: a base structure and a sensing structure. The base structure is formed by a textured nylon filament yarn (75 dtex, 36f) and a nylon-covered spandex yarn (50 dtex). The sensing structure is composed of a silver-coated nylon filament yarn (40 dtex, 12f) and the same nylon-covered spandex yarn. The sensing area can be designed in a single area (single sensor) or multiple areas (multiple sensors). For the swallowing measurement, three sensor areas are designed, each having two leads for hardware connection. These leads are the same conductive yarn used in the sensing area but knitted in the fabric base structure as a long single course of loops. After making up a sensing collar through a cut-and-sew process, metal snap buttons are attached onto each end of the leads as connecting points for the MCU unit (FIG. 5A).

In some aspects, the present invention relates to a wearable and Bluetooth-ready mini MCU device that is attached to the sensing collar to form a complete wearable device. This hardware performs collection of the strain sensor signals, A/D conversion, and data transmission through an embedded Bluetooth module. The system involves a low power Bluetooth LE module that minimizes power consumption and can handle the small size of data (~1 Kb/s). A nRF51822 system-on-a-chip (Nordic Semiconductor) is connected to a microcontroller (Arduino Pro Mini), and powered by a 400-hr battery. Data is acquired at 40 Hz. An Android smartphone is used to collect the data over a Bluetooth LE connection. The microcontroller sends the raw sensing data to the Android device or laptop receiver where the data is processed. The system is smaller than a quarter, be unobtrusively worn in a shirt collar, and easily removed for washing.

The signal is processed at the receiver end starting with a three-point running average filter with a five-second sliding window for analysis. Data is processed in a machine learning classifier described below.

Fabric Sensor Performance Evaluation and Test Method Development: an engineering evaluation of the specifications of the sensor is performed. Battery life is evaluated under continuous use, repeatability is measured using a plunger normal to the surface of the sensor, cycle life is measured using the same plunger, and bandwidth is measured by vibrating the plunger with a voice coil.

Fabric Sensor Measurement Validation through Human Subject Study: Three (3) equal groups of 15 healthy individuals were recruited. One group (young children) was from 4-8 years old, another (young adult) was from 18-26 years old, and a third (older adults) was from 65-85 years old. A proportionate number of each gender and race/ethnicities for Austin were recruited. Due to the innovative nature of the signals from the fabric sensor, this preliminary validation data was used to perform power analyses to inform sample sizes for future research projects.

Each subject was fitted with one of the appropriately sized prototype shirts. The test was composed of numerous conditions in randomized order, including: drinking water from a cup or measured by a syringe, drinking thickened liquid from a cup or measured by a syringe, taking bites of applesauce, pudding, and/or graham crackers, clearing the throat, coughing, inhaling and exhaling deeply, moving the neck side-to-side and up-and-down, vocalizing “ahh” and reading a short paragraph. Instead of reading, young children were asked to recite a poem. The swallows were validated using submental electromyographic (EMG) activity measurement and nasal cannula measurement as ground truth.

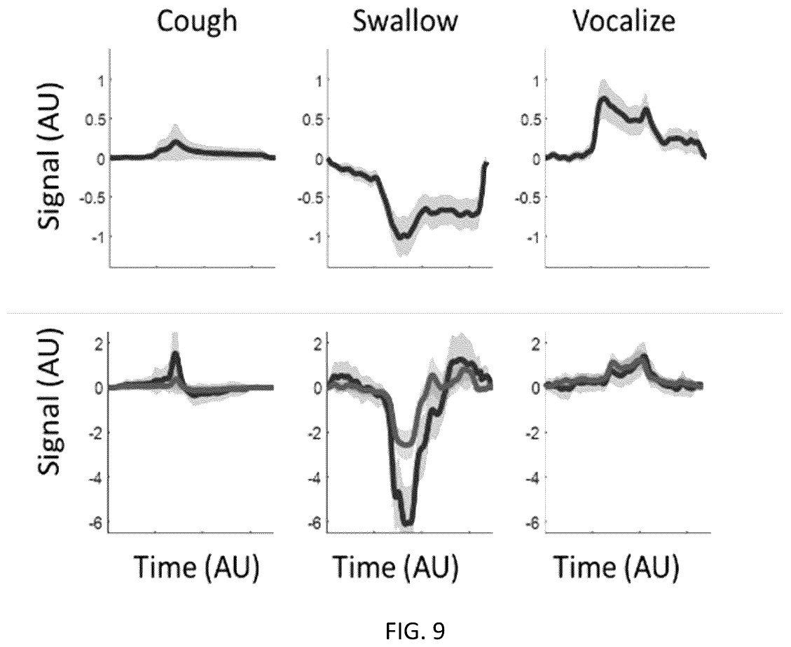

Based on previous work [Santoso L F, Baqai F, Gwozdz M, Lange J, Rosenberger M G, Sulzer J, et al. Applying Machine Learning Algorithms for Automatic Detection of Swallowing from Sound. In: 2019 41st Annual International Conference of the IEEE Engineering in Medicine and Biology Society (EMBC). IEEE; 2019], a neural network was used, using the scaled conjugate gradient (SCG) method [Moller M F. A scaled conjugate gradient algorithm for fast supervised learning, Neural Networks. 1993; 6(4): 525-33]. This method minimizes an error function by altering the weight vector of the neural network using negative first order gradient descent and zeroes the second order approximation. A hidden network of 100 nodes was used. 70% of the data was used for training, 15% for testing, and 15% for validation per fold, with a five-fold cross-validation. The main outcome measure was the mean accuracy of the classifier. The data was evaluated as a binary classifier (swallow vs. not swallow). FIG. 9 shows preliminary data based on a 23-year-old woman (top row) and a 61-year-old woman (bottom row) to illustrate the ability to differentiate swallowing from other activities. There is a clear distinction between swallowing (middle column) from coughing and vocalizing (left and right columns) using the methods described above.

Furthermore, each participant was engaged in a brief interview about their experience with wearing the prototype shirts. Such human factors data are used to improve upon the design of the sensor and sensor garments.

Performance and Reliability Metrics/Standards: the prototyped fabric sensor was characterized in terms of material durability, sensing performance related to sensitivity, retention and prediction accuracy, and comfort and aesthetic properties.

Mechanical Properties: the sensing fabric strength was tested using the ball burst test method defined by ASTM D3787. The sensor area abrasion resistance was evaluated in accordance with ASTM D3884.

Sensing Performance: the sensor linear correspondence vs. tensile strain was characterized using the grab test method for tensile strength and elongation of textile fabrics described in ASTM D5034. Repeated stretching and washing may affect the retention of the sensor sensitivity expressed by Gauge Factor (GF). Resulted variation from frequent tensile deformation is estimated by a cyclic tensile test based on the same instrumental setting of ASTM D5034. Retention of the sensor sensitivity subject to repeating wash is evaluated using a home laundering method defined in AATCC 135.

Sensing Reliability: measurement accuracy for swallow prediction was assessed in two phases. In the lab testing phase, it was estimated in a signal process by counting the percent of correctly identified swallow movements in a given range of signals. In the human subject test phase, it was evaluated by a parallel comparison between the data obtained from an EMG method and the results measured by the fabric sensor.

Comfort and Aesthetic Properties: wear comfortability and aesthetics of the fabric sensor device during swallowing measurement or monitoring were evaluated in the human subject test phase. A survey form was provided to participants in this test to collect their subjective feedback on the wearing comfort and aesthetic levels they experienced.

Key Target Specifications: important specifications for the design and prototype of the fabric sensor device are listed in the following Table 1.

TABLE 1
Key Target Specifications

| Component/Property | Requirement |
|---|---|
| Conductive Yarn | Silver-coated nylon filament 40 dtex, 12f; specific |
| | resistance 1250 Ω/m; resistivity 0.1 ± 0.05 Ω*m; |
| | tenacity 5.6 cN/dtex |
| Base fabric yarns | Texture nylon filament 75 dtex, 36f; spandex 50 |
| | dtex covered by 20 dtex nylon |
| Fabric loop density | 26 wales/cm; 17 courses/cm |
| Fabric burst strength | Ball burst test minimum force 30 lbf |
| Sensing collar dimension | L = 350 mm ; H = 100 mm |
| Sensor area and location | a = 15 mm; b = 50 mm; c = 110 mm; d = 5mm |
| Sensor specification | Initial resistance 5 Ω; impedance range 1-2000 Ω; |
| | sampling rate 10-40 Hz; transmitting rate 20/s |
| Sensor sensitivity & | GF ≥ 25; washing time ≥ 100; cyclic stretching |
| retention | time ≥3000 |
| Measurement accuracy | 95% |
| Comfort & aesthetics | Accepted |
| MCU device | Arduino Pro Mini with nRF51822 |

Risk Assessment and Mitigation Plan: based on the hands-on experience in the fabric sensor research, possible risks for this research investment could be envisaged in the follow aspects. Accordingly, alternative approaches to mitigate these risks are also proposed.

There is the potential that neck motion during swallowing could affect sensor accuracy. This is a critical issue for any swallowing measurement system. The disclosed system comprises a multi-sensor array, and so is configured to filter out neck motions by examining relative strain between sensors. There are also other design adaptations to create arrays that examine medial-lateral strain, which would also help filter out non-swallowing motions. Over repeated usage, the sensor and collar may in some embodiments show signs of wear and reduced elasticity. Aside from simply replacing the shirt or collar, an elastic drawstring may be used to modulate sensitivity. Furthermore, there is a potential risk that the fabric sensors will not be sensitive enough to register reliable signals on individuals with small larynges, such as children and/or women. In this case, the garments may be adjusted to fit each individual.

The experiment recruited small children who may have limited ability to follow instructions or patience to conduct a full experiment. The experiment was adapted as much as possible to accommodate the children's comfort, including using juice for swallowing and incorporating simple games to encourage vocalizing and coughing.

In the situations where algorithms insufficiently classify swallowing from non-swallowing behavior, in some embodiments, separate classifiers may be generated based on age group or based on one individual's swallowing signals.

The disclosed fabric strain sensor system was developed in a laboratory environment. The baseline of this technology departure is MRL/TRL 4. In the pilot study, the experimental fabric strain sensor was applied to successfully detect swallowing, coughing, and vocalizing from measured larynx movements. The disclosed fabric sensing technology has the potential to open up new opportunities to produce a transformative and cost-effective swallow measurement device ideally integrated into garment products.

With an MCU, the disclosed knitted strain sensor was used to measure throat area surface strain generated by three larynx movements: swallowing, coughing, and vocalizing. As shown in FIGS. 9 and 10, the tested results from a single fabric sensor clearly differentiated the three throat movements. The project report concluded that the knitted strain sensor was capable of detecting throat movements. Later, continuous work was carried on using two sensors simultaneously to measure laryngeal movement in swallowing in order to obtain as much data as possible, and on refining the MCU device and code to develop physical models for accurate swallow prediction based on machine learning algorithms.

The creation of this fabric sensing technology includes the strain sensor fabric, mechatronic signal acquisition, testing procedure, and data analysis for parameter extraction and swallow decoding. Pilot data reveals that the fabric strain sensor can identify swallowing consistently in comparison to vocalization and coughing (FIG. 10).

### Example 2: Knitted Strain Sensor Device Used for Pharyngeal Rehabilitation

The goal of the disclosed experiment was to develop a wearable sensing device able to diagnose and monitor swallowing behavior of individuals with swallowing dysfunction known as dysphagia. The invented technology comprises a fabric sensing device capable of measuring physiological signals generated by swallowing, coughing, and vocalizing. In some embodiments, the device includes a wearable fabric collar with knitted-in strain sensors, hardware for signal acquisition and wireless transmission, firmware for data logging and display, and a patient software app compatible with computers or smart phones for swallow monitoring, as shown in FIG. 5A. The sensing collar may be worn around a subject's neck, tightly covering the major area around the laryngeal prominence. Swallowing signals may be collected by a mini microcontroller unit (MCU), transmitted wirelessly by Bluetooth, and subsequently displayed on a laptop. Each device component is described individually as follows.

Fabric sensing collar: an exemplary fabric sensing collar is made from a knitted structure composed of a stable and stretchy fabric base and three strain sensing areas. The base structure is formed by a textured filament yarn and a nylon-covered spandex yarn. The sensing structure is composed of a silver-coated nylon filament yarn. Each of the three sensor areas has two leads for hardware connection. These leads are either knitted into or sewn into the base fabric with a conductive yarn. After fabricating a sensing collar through a cut-and-sew process, metal snap buttons may be attached onto each end of the leads as connecting points for a hardware unit.

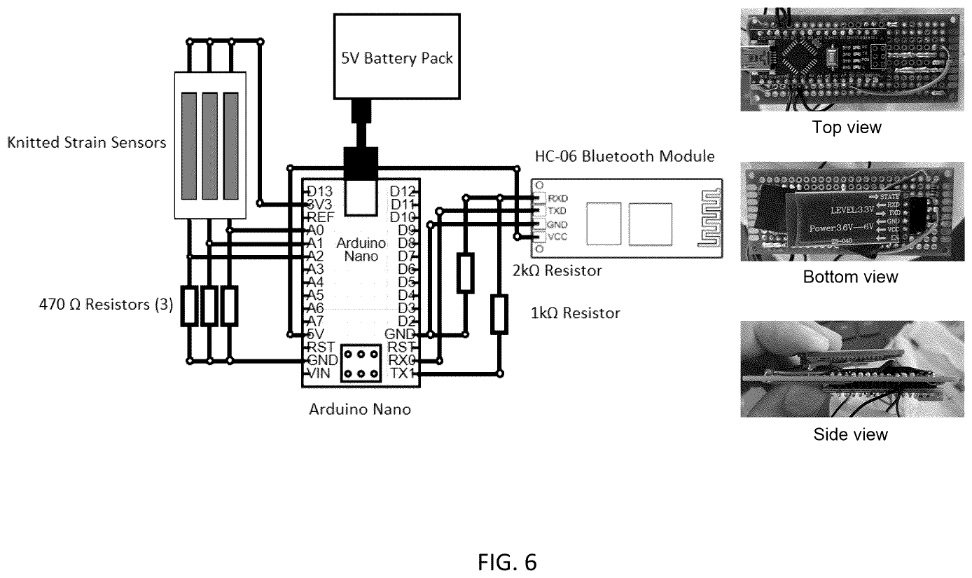

Hardware configuration: the device hardware of the depicted example is constructed as shown in FIG. 6. It includes an MCU, a Bluetooth module for wireless data transfer, and a 5V battery for a power supply. The MCU is a smallest and complete Elegoo Nano Board based on the ATmega328P and a mini USB cable, compatible with the Arduino Nano V3.0. The Bluetooth module is a HiLetgo HC-06 RS232 Wireless Bluetooth Serial RF Transceiver with a 4-pin bi-directional serial channel slave mode for Arduino. It works with any USB Bluetooth adapters. It features a small size, low power consumption, and high sensitivity for sending and receiving signals. The Bluetooth version is V2.0+EDR. The operating voltage is 3.3V.

System Description: Power Supply: 5V Battery Pack, Arduino Nano: The Nano can be powered via USB connection, 6-12V unregulated external power supply (VIN pin), or 5V regulated external power supply (5V pin). HC-06 Bluetooth module: The HC-06 will work with a supply voltage of 3.6V to 6V, however, the logic level of the RX pin is 3.3V and is not 5V tolerant.

Knitted Strain Sensors System: Components: Arduino Nano: The depicted example system used the Arduino's multi-channel, 10-bit analog-to-digital converter to map input voltages between 0V and the operating voltage (3.3V) into integer values between 0 and 1023. The analog-to-digital converter is used to read in the variable resistances of the knitted strain sensors. Resistors: To turn each knitted strain sensor's variable resistance into a readable voltage, a static 4700 resistor is included to create a voltage divider. Voltage divider: In the example system, a varying voltage was produced from a varying resistance with a voltage divider composed of the varying resistance (knitted strain sensor) and a fixed resistance (47002 resistor).

By observing the voltage at the circuit node (Arduino's analog pins) where the two resistors are connected together, a varying integer serial output is seen, and with some math, the corresponding variable resistance of the strain sensor is calculated. Knitted Strain Sensors: An output voltage is created by the variable stretching of each knitted strain sensor to allow for the Arduino IDE to process the real-time voltage signals during testing.

Connection (Knitted Strain Sensors to Arduino Nano): with reference to FIG. 6, in the depicted example, the system was configured with the following electrical connections: Top sensor (variable resistor): 3.3V—top sensor—A0 pin—470 Ω—GND, Middle sensor (variable resistor): 3.3V—middle sensor—A1 pin—470 Ω—GND, Bottom sensor (variable resistor): 3.3V—bottom sensor—A2 pin—470 Ω—GND.

Bluetooth Connection System: Components: HC-06 Bluetooth Module: The class 2 slave Bluetooth module uses serial protocol for transparent communication. The HC-06 acts as a serial port through which data is sent or received. When using a serial terminal or a Bluetooth customized GUI on a computer, one can monitor the knitted strain sensors' outputs. When the module receives wireless data, it is sent to the Arduino through the TX pin of the HC-06 (connected to the RX pin of the Arduino). No user code specific to the Bluetooth module was used in the example microcontroller program. Arduino Nano: The Bluetooth module occupies a serial port into which data is fed. The Arduino supplies the module with a 5V input; with its RX pin receiving serial data from the Bluetooth module and the TX pin transmitting serial data to the Bluetooth module. Resistors: 1 kΩ and 2 kΩ resistor are included in the HC-06 RX pin connection to create a voltage divider; bringing the Arduino's 5V down to 3.3V.

Voltage divider: The output of the Arduino is a 0-5V logic level. The HC-06 requires a 0-3.3V logic level. The two included resistors divide the 5V high level to create a voltage at the RX pin of 3.3V. The divider must be connected to ground in order to work.

Connection (Bluetooth module to Arduino Nano): VCC—5V pin (supplied by 5V battery pack), GND—GND pin, RX via a voltage divider—TX pin, TX—RX pin High Performance Desktop/Laptop: when viewing the Bluetooth-transmitted serial data, a high-speed desktop/laptop was used to prevent lag of real-time data display and poor communications.

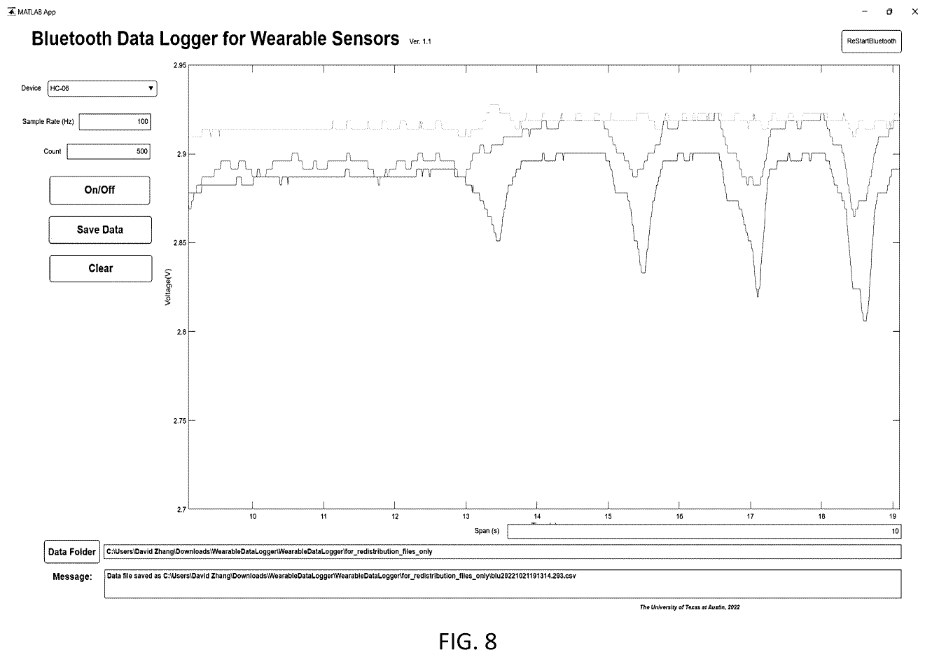

MATLAB GUI: for the fabric sensing data acquisition and display, A GUI window was created using MATLAB, as shown in FIG. 8. This allows users to control testing and save measured data.

Preliminary Test Results: Testing Procedure 1:
1. For the 3-sensor test, the laryngeal prominence was placed directly on the middle sensor with the other two sensors (top and bottom sensor) accommodating for movements above and below the laryngeal prominence.
2. The hardware and data acquisition program was then initialized.
3. The garment was then fit onto the neck of the subject, placing the sensors onto the designated areas of the neck.
4. When test subject was ready, the data acquisition program was started and the subject was directed to perform the following activities:
   a. Sit still for approx. 5 seconds to allow for lag,
   b. Have the subject repeat the specified action 3 times,
   c. Once subject finishes, record for another 5 seconds,
5. The data collection was then ended and the data saved as a CSV,
6. The above steps were repeated until all actions were collected.

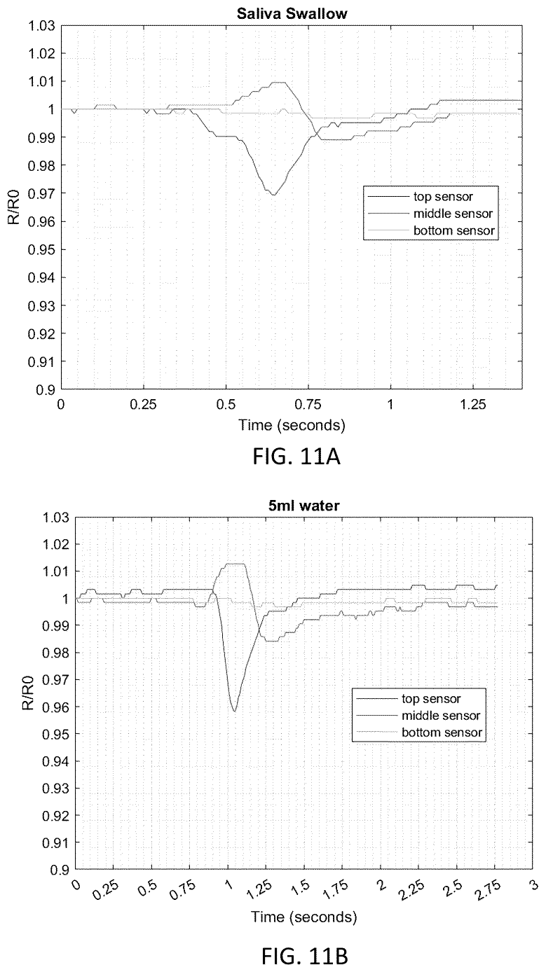

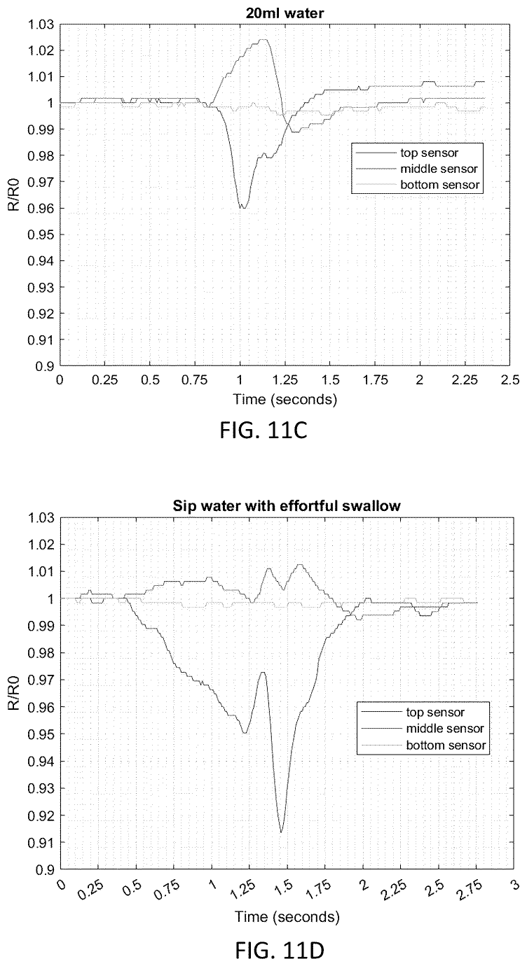

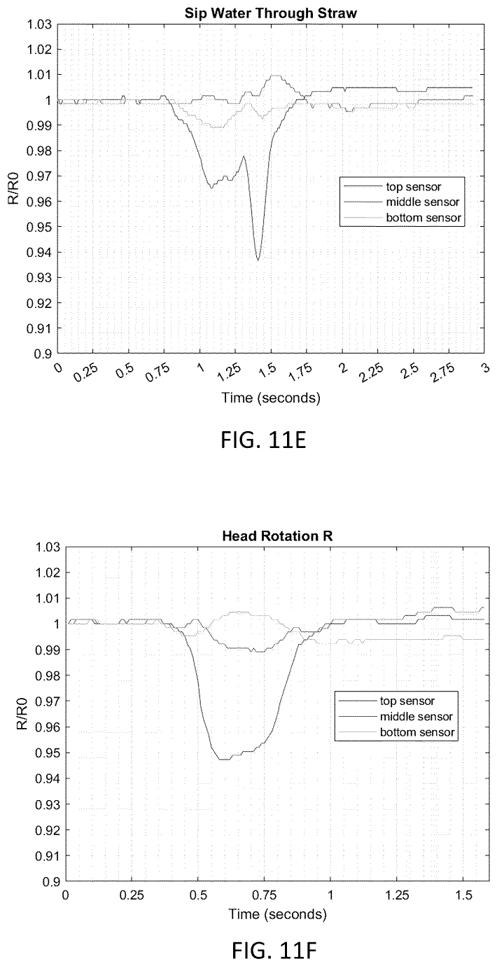

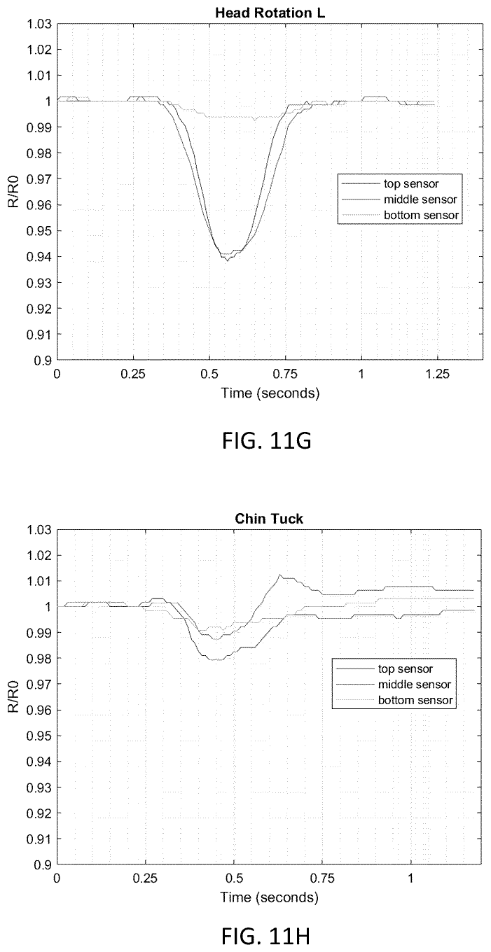

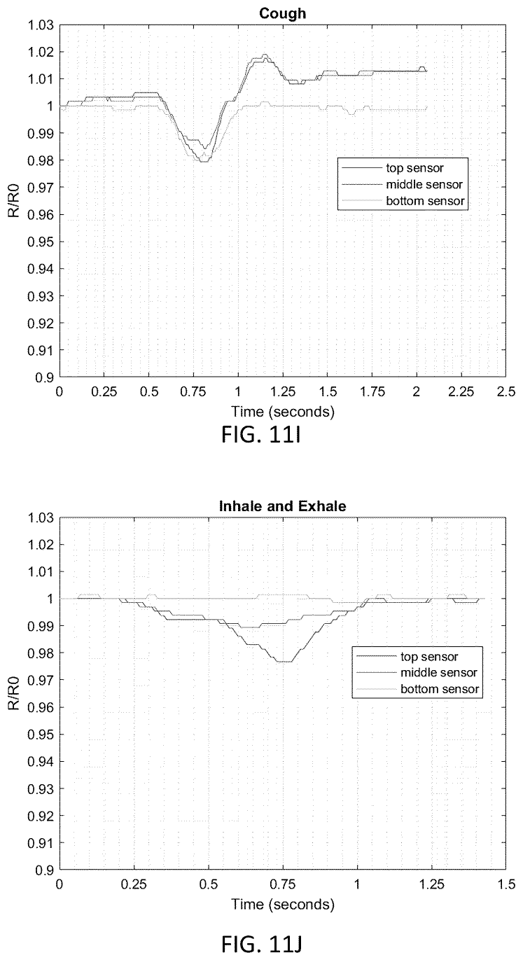

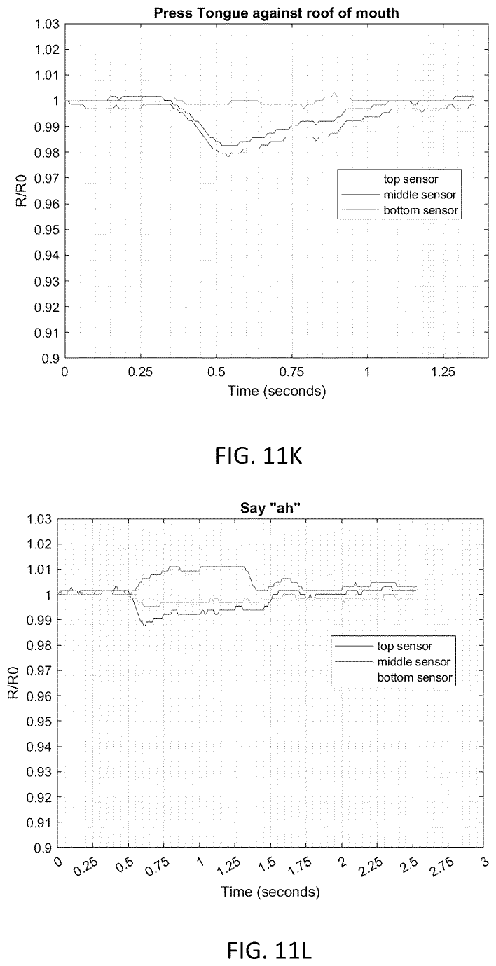

The obtained preliminary test data indicated that all three fabric sensors could generate meaningful signals from swallowing and head moving. Results from the preliminary test data are found in FIG. 11A through FIG. 11L. The signal samples provided distinct signal patterns related to differentiate between water swallowing, head moving, coughing and inhaling/exhaling, and vocalizing. The signal samples revealed that Top Sensor and Middle Sensor produced significant signals in all swallowing, coughing, vocalizing, and head moving, while the Bottom Sensor generated significant signals mainly in head moving only. This meant that the Bottom Sensor could be used to monitor head movement, so that subjects' swallowing processes could be identified more accurately.

### Example 3: Swallow Sensing Collar Dimensions and Approach for Swallow Diagnosis

A swallow dataset was then created for data analysis and swallow prediction model development. The approach taken is illustrated in FIG. 12. First, measured swallowing signals were analyzed to extract characteristics to define a swallow vector. Then, multivariate analysis and neural network computing were used to determine the best algorithm for swallowing recognition and classification. After establishing a prediction model, a computer program was developed to implement a real-time and online computation for incoming swallow signals, together with a user-interface app for display of analyzed results.

FIG. 5B depicts an exemplary knitted strain sensor device according to aspects of the present invention. Shown in FIG. 5B are various positions and dimensions for an exemplary knitted strain sensor device having at least one collar and at least one button. In some embodiments, the at least one collar has a length (L) and a height (H). Also shown in FIG. 5B are other positions and dimensions as set forth in Table 2 below.

TABLE 2
Measurement of Sensing
Collar Dimensions and Sizes

| | Collar 1 | Collar 2 |
|---|---|---|
| Location | (mm) | (mm) |
| L | 587 | 600 |
| H | 71 | 72 |
| M | 140 | 140 |
| N | 125 | 125 |
| a | 13 | 12 |
| b | 54 | 50 |
| c | 7 | 7 |
| d | 2 | 11 |
| e | 20 | 19 |
| f | 23 | 23 |
| g | 15 | 15 |
| h | 20 | 20 |
| i | 100 | 100 |
| j | 55 | 55 |
| k | 40 | 40 |

TABLE 3
Measurement of Button Position,
Diameter and Circumference

| | Button | Diameter | Circumference |
|---|---|---|---|
| Collar | Position | (mm) | (mm) |
| 1 | P1-S1 | 138 | 432 |
| 1 | P1-S2 | 131 | 412 |
| 1 | P1-S3 | 125 | 392 |
| 1 | P1-S4 | 118 | 372 |
| 1 | P2-S1 | 125 | 392 |
| 1 | P2-S2 | 118 | 372 |
| 1 | P2-S3 | 112 | 352 |
| 1 | P2-S4 | 106 | 332 |
| 2 | P1-S1 | 144 | 451 |
| 2 | P1-S2 | 137 | 431 |
| 2 | P1-S3 | 131 | 411 |
| 2 | P1-S4 | 125 | 391 |
| 2 | P2-S1 | 131 | 411 |
| 2 | P2-S2 | 125 | 391 |
| 2 | P2-S3 | 118 | 371 |
| 2 | P2-S4 | 112 | 351 |

### Example 4: The Development of a Wearable Sensing Device for Swallow Monitoring and Classification

The disclosed example presents the development and evaluation of a non-invasive, wireless wearable sensing device (e.g., a knitted strain sensor system) for continuous monitoring and classification of swallowing and non-swallowing behaviors in healthy individuals. The device employs knitted strain sensors for enhanced comfort and seamless integration into daily life. Using machine learning algorithms, swallowing patterns were identified to provide insights into the behaviors' features and characteristics. Data collected from three strategically placed sensors were processed and analyzed, with feature extraction and selection methods employed to optimize input for the machine learning models. The fine k-Nearest Neighbors (KNN) model demonstrated superior performance, achieving an accuracy of 75.6%, precision of 72.4%, sensitivity of 81.5%, specificity of 74.4%, and F1-score of 76.7%. Results indicated that incorporating multiple sensors improved classification accuracy, with the highest accuracy achieved using three sensors (75.6% with 51 features). The wearable sensing device and accompanying algorithms show potential in dysphagia diagnosis, treatment, and monitoring, in hopes of paving the way for further refinement and potential applications in other aspects of dysphagia care, such as swallow therapy and telemedicine integration.

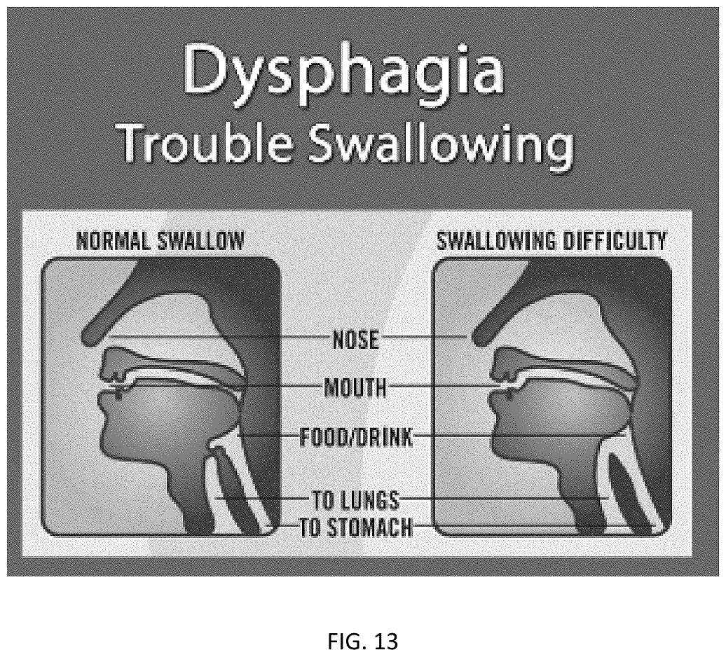

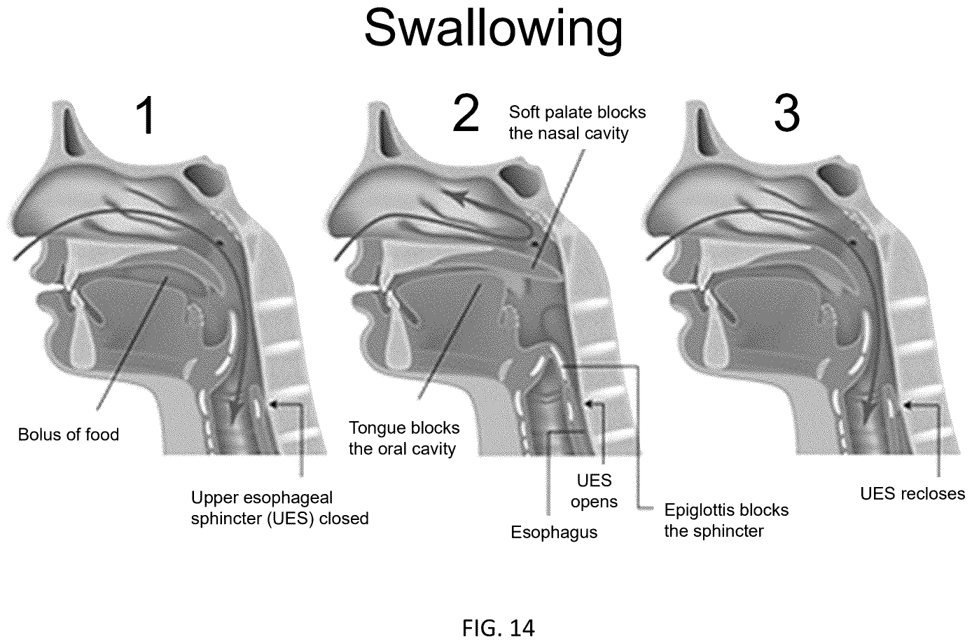

Swallowing is an intricate physiological process that plays a vital role in human nutrition and health (FIG. 13—Comparison between a healthy and unhealthy swallow (Mohannak, Pattison, Hird, & Needham, 2019)). Dysphagia, or difficulty swallowing, is a pervasive condition affecting a significant portion of the population, with approximately 500,000 children and 9 million adults suffering from this issue in the United States alone (Bhattacharyya, 2014; Borowitz & Borowitz, 2018). Swallowing disorders can arise due to a variety of causes, including neurological damage from cerebral palsy, stroke, Parkinson's disease, or traumatic brain injury (González-Fernández et al., 2013; Heuschmann, 2004; Sörös et al., 2009). Dysphagia can have severe consequences such as aspiration, malnutrition, dehydration, and even death, highlighting the importance of early detection and monitoring (Marik & Kaplan, 2003; Rofes et al., 2018).

The conventional approach to evaluating swallowing in clinical settings involves visual examination or manual palpation, but these methods can be inaccurate and are subject to variability between clinicians (Marik & Kaplan, 2003; Sulzer & Karfeld-Sulzer, 2021). As a result, more objective, non-invasive techniques have been sought, such as video fluoroscopy and fiberoptic endoscopic evaluation of swallowing (FEES), although these are expensive, time-consuming, and often inaccessible in non-clinical environments (Aviv, 2000; Martin-Harris et al., 2008a). In recent years, advances in wearable technologies have shown promise in addressing these limitations, particularly in the area of biosensing for health monitoring (Patel et al., 2012). Wearable devices offer several advantages, including continuous and real-time data collection, improved patient compliance, and a greater capacity for remote monitoring and personalized care (Patel et al., 2012). However, existing solutions are primarily designed for adults and may not be suitable for children who have distinct anatomical, physiological, and behavioral requirements (Patel et al., 2012). Additionally, usability is a critical consideration for pediatric populations, as devices must be affordable, comfortable, durable, easy to use, and able to integrate seamlessly into daily activities (Sulzer & Karfeld-Sulzer, 2021).

The motivation behind this research lies in the development of a fabric-based, wearable sensing device for the non-invasive monitoring and classification of swallowing behavior in children and adults, particularly those at risk of aspiration due to dysphagia. Building upon previously disclosed work (Li et al., 2021), this project aims to create a low-profile, comfortable, and easily integrated solution using knitted strain sensors, incorporating silver-coated yarn to form the sensing areas (FIG. 1C). This design has been shown to exhibit high sensitivity and rapid response times for detecting surface strain on both 2D and 3D surfaces (Li et al., 2021). In addition to providing a non-invasive and cost-effective alternative to traditional assessment methods, the proposed wearable sensor offers a multitude of other benefits. By enabling continuous, real-time monitoring, it can facilitate early detection of dysphagia and improve patient outcomes. The sensor's low-profile design and integration with existing clothing can increase user acceptance and reduce the burden of remembering to carry or retrieve the device for daily use. Furthermore, its compatibility with smartphone apps allows for easy data transmission and analysis, promoting collaboration between patients, caregivers, and healthcare providers. Through the development of this innovative wearable sensing device, this research aims to address the pressing need for accessible, reliable, and user-friendly solutions to monitor and manage swallowing disorders in pediatric and adult populations.

Swallowing is a complex process, encompassing a sequence of precisely coordinated events that involve both the central nervous system and the muscles of the upper aerodigestive tract. It can be divided into three primary phases: the oral phase, the pharyngeal phase, and the esophageal phase (FIG. 14) (Ertekin & Aydogdu, 2003).

The oral phase involves manipulating food in the mouth to create a cohesive bolus and propel it towards the back of the oral cavity. The tongue plays a vital role in this phase, as it forms a seal with the hard palate, ensuring the bolus remains within the oral cavity and does not enter the pharynx prematurely. This phase is mainly voluntary and is controlled by the somatic nervous system (Logemann, J., 1984).

The pharyngeal phase commences when the bolus reaches the posterior part of the oral cavity and activates the swallow reflex. The soft palate elevates and closes the nasopharynx, preventing nasal regurgitation. The larynx elevates and closes, protecting the airway from aspiration. The epiglottis folds down over the laryngeal opening, further ensuring airway protection. The upper esophageal sphincter (UES) relaxes, allowing the bolus to enter the esophagus. This phase is involuntary and is coordinated by the central pattern generator in the brainstem, which controls the sequence and timing of muscle contractions (Logemann, J., 1984).

The esophageal phase is responsible for propelling the bolus from the UES to the stomach through peristaltic waves. The lower esophageal sphincter (LES) opens to allow the bolus to enter the stomach and closes afterward to prevent reflux. This phase is also involuntary and is regulated by both the enteric nervous system and the central nervous system (Logemann, J., 1984).

Swallowing disorders, or dysphagia, can occur due to a variety of factors, including neurological diseases (e.g., stroke, Parkinson's disease), structural abnormalities, muscle weakness, or injury to the nerves and muscles involved in swallowing. Dysphagia can lead to severe complications, including aspiration pneumonia, malnutrition, dehydration, and decreased quality of life (Logemann, 1998).

For a wearable device to accurately classify patterns and detect swallowing abnormalities, it must be sensitive to the key biomechanical events that occur during the swallowing process. These events include laryngeal elevation, hyoid bone movement, and UES opening, all of which can be observed through the detection of muscle contractions or movement patterns. Surface electromyography (sEMG) can be employed to monitor muscle activity, while accelerometers, gyroscopes, and strain sensors can be used to detect movements associated with swallowing.

To enhance the accuracy and reliability of the device, it is crucial to consider factors such as sensor placement, signal-to-noise ratio, and the need for filtering and preprocessing of the acquired data. The selection of appropriate machine learning models for pattern recognition and classification is also essential, as it can significantly impact the device's ability to detect and distinguish between swallowing and non-swallowing events.

Overall, understanding the biomechanics of swallowing and the factors that contribute to dysphagia is crucial for the development of a wearable device capable of accurately classifying swallowing patterns. By focusing on key events during the swallowing process and incorporating appropriate sensor technologies, data preprocessing, and machine learning models, a wearable device can potentially contribute to the early detection and management of swallowing disorders in both pediatric and adult populations.

Machine learning (ML) has gained significant momentum in recent years, becoming an integral part of modern healthcare systems. This technology leverages algorithms and statistical models to analyze data, identify patterns, and make predictions. The application of machine learning in healthcare has the potential to transform various aspects of patient care, from diagnosis and treatment planning to management of chronic conditions and personalized medicine (Esteva et al., 2019).

One of the critical applications of machine learning in healthcare is the early detection and diagnosis of diseases. For example, ML algorithms have been utilized in the analysis of medical images, such as X-rays, MRIs, and CT scans, to identify subtle patterns that might be indicative of pathological conditions, like cancer (Litjens et al., 2017). This approach has shown promising results in improving the accuracy of diagnosis, thereby facilitating better treatment outcomes (Gulshan et al., 2016). Moreover, ML can aid in the development of predictive models for patient risk stratification, helping clinicians identify high-risk individuals and tailor treatment plans accordingly (Obermeyer & Emanuel, 2016).

Another area where machine learning has shown promise is in the personalization of medical treatment. ML algorithms can analyze a patient's genetic information, lifestyle factors, and clinical data to identify individual patterns and predict their response to specific therapies (Collins & Varmus, 2015). This enables healthcare professionals to develop personalized treatment plans that are more likely to be effective for the patient, reducing the likelihood of adverse side effects and improving overall outcomes (Jameson & Longo, 2015).

Machine learning has also been applied in the management of chronic diseases, such as diabetes and cardiovascular disorders. By analyzing large datasets from wearable devices and other sources, ML algorithms can identify patterns and provide real-time feedback to patients and healthcare providers. This approach can help patients maintain better control over their conditions and promote more effective disease management strategies (Steinhubl et al., 2015).

In the context of dysphagia assessment, machine learning has been employed to analyze data from various sources, such as videofluoroscopy, FEES, and surface electromyography (sEMG) (Takahashi et al., 1994). By leveraging ML algorithms, researchers have been able to develop models for classifying swallows, identifying swallowing abnormalities, and predicting patient outcomes. The integration of machine learning with wearable sensors for swallow monitoring offers exciting possibilities for non-invasive, continuous assessment and real-time feedback, ultimately leading to improved patient care and outcomes.

Some recent studies have focused on the use of machine learning algorithms in the analysis of sEMG signals to differentiate between normal and abnormal swallows. For instance, researchers have utilized various ML techniques, including support vector machines (SVM), artificial neural networks (ANN), and random forests (RF), to classify swallows based on the recorded sEMG data (Zoratto et al., 2010). These approaches have shown promising results in improving the accuracy and reliability of swallow classification, highlighting the potential benefits of integrating ML with wearable sensor technology in dysphagia assessment.

In summary, machine learning has demonstrated significant potential in various healthcare applications, from early disease detection and diagnosis to personalized medicine and chronic disease management. The integration of ML with wearable sensor technology for swallow monitoring offers promising possibilities for non-invasive, continuous assessment of dysphagia and improved patient outcomes. As research in this area progresses, it is likely that machine learning will play an increasingly critical role in the development and implementation of innovative healthcare solutions.

Wearable Fabric Device: The disclosed wearable fabric collar (i.e., knitted strain sensor system), designed to provide a comfortable and non-intrusive solution for detecting swallowing events, especially in children, comprises a knitted structure comprising a stable and stretchy fabric base and three strain sensing areas (FIG. 1A). The base structure is formed by a textured filament nylon yarn (75 dtex, 36f) and a nylon-covered spandex yarn (50 dtex), ensuring comfort and adaptability to various neck sizes and shapes. This knitted fabric strain sensor structure has been used in a previous study (Li et al., 2021) for strain sensing. In the study conducted by Li et al. (2021), the knitted fabric strain sensor (in some examples referred to as just a knitted strain sensor) was tested in a different scenario. It was strategically placed on the knees of a user to record and output biosignals during activities such as walking, running, climbing stairs, and descending stairs. With the different activities displaying distinct signal signatures through their knee outputs, this showcased the sensor's adaptability and ability to accurately capture strain changes across various activities.

In contrast, the knitted fabric sensor has adapted and employed this versatile fabric sensor structure in our wearable collar, specifically targeting the detection and monitoring of swallowing events. This innovative application extends the sensor's utility, demonstrating its potential in the healthcare sector.

Incorporated into the base fabric, three strain sensing areas utilize a silver-coated nylon filament yarn (40 dtex) that provides electrical resistance data for detecting body motions related to swallowing. Each of the three strain sensors is strategically placed to capture the movement in the larynx (Adam's apple). The middle sensor is placed directly on the larynx, the top sensor above the larynx but below the chin, and the bottom sensor below the larynx to capture head and body movements (FIG. 2B). The disclosed design allows for the efficient detection of swallowing events, with a reported strain sensing sensitivity reaching 60% for uniaxial tension in the 2D fabric surface, and 120% for all-direction tension in the 3D fabric surface. The response time for the fabric strain sensors is 400 ms for the 2D fabric surface and 350 ms for the 3D fabric surface (Li et al., 2021). These performance characteristics make the wearable fabric collar a promising solution for real-time monitoring of swallowing events in both adults and children.

Moreover, the wearable fabric collar is easily connected to the hardware for signal acquisition and data logging. Metal snap buttons are attached to the leads at the ends of the strain sensors, serving as connection points for the mini microcontroller unit (MCU) (Li et al., 2021). This ease of connection ensures that the wearable collar can be comfortably and securely worn by users during monitoring.

Hardware components: The device hardware includes a mini microcontroller unit (MCU), a Bluetooth module for wireless data transfer, and a 5V battery pack for power supply. The MCU is a compact Elegoo Nano Board based on the ATmega328P, compatible with Arduino Nano V3.0. This board is a powerful yet small component, capable of performing complex data processing tasks and providing the foundation for the system's operations (FIG. 6).

The wireless transmission is handled by a HiLetgo HC-06 RS232 Wireless Bluetooth Serial RF Transceiver. This module features a 4-pin bi-directional serial channel in slave mode and is compatible with Arduino-based systems. It works with any USB Bluetooth adapter and offers low power consumption, high sensitivity, and small form-factor. The Bluetooth version is V2.0+EDR, and its operating voltage is 3.3V. This module enables efficient wireless data transfer between the wearable device and the user interface, allowing for real-time monitoring and analysis of the collected swallowing data.

The power supply for the device is provided by a 2000 mAh TOPUSSE Lithium-Ion Polymer battery, ensuring continuous and stable operation during testing. The Arduino Nano can be powered via a USB connection, a 6-12V unregulated external power supply (VIN pin), or a 5V regulated external power supply (5V pin). The HC-06 Bluetooth module requires a supply voltage of 3.6V to 6V, and its RX pin logic level is 3.3V, which is not 5V tolerant. These power requirements must be considered when designing the power supply system for the device to ensure optimal performance and component longevity.

To integrate all hardware components, they were soldered onto a printed circuit board (PCB) breadboard. This PCB serves as the backbone for the hardware configuration, facilitating electrical connections between the components and providing a compact, organized platform for the entire system. Proper design and layout of the PCB are crucial for minimizing electrical interference and ensuring optimal performance of the device.

Software components: Disclosed herein are various software elements used in conjunction with the hardware for the wearable sensing device are crucial for data acquisition, processing, and presentation to the user, ensuring seamless and effective operation of the system

To facilitate data acquisition and display, a Graphical User Interface (GUI) is developed using MATLAB's App Designer. The GUI allows users to control the testing process and save the measured data from the knitted strain sensors, providing an interactive platform for the real-time monitoring of swallowing activities (FIG. 8).

The Arduino Nano, equipped with a multi-channel, 10-bit analog-to-digital converter, is utilized to map input voltages from the knitted strain sensors into integer values. Voltage dividers, formed by a combination of the variable resistance of the strain sensors and a fixed 47002 resistor, allow the MCU to read the variable resistances as voltage signals. This voltage signal is then processed by the Arduino IDE during testing, enabling real-time monitoring of the sensor outputs.

For wireless communication between the wearable device and the user interface, the HC-06 Bluetooth module is used. This class 2 slave Bluetooth module employs a serial protocol for transparent communication. By using a serial terminal or a customized GUI on a computer, users can control and monitor the outputs from the knitted strain sensors. The Bluetooth module requires no user-specific code, allowing seamless integration with the Arduino-based system.

A high-performance desktop or laptop is needed to view the Bluetooth-transmitted serial data, ensuring minimal lag during real-time data display, and preventing any communication issues. The Arduino IDE code provided is designed to read analog inputs on pins A0, A1, and A2, corresponding to the top, middle, and bottom strain sensors, respectively. The sensor values are printed to the Serial Monitor and can also be visualized using the Serial Plotter (Tools>Serial Plotter menu) for graphical representation.

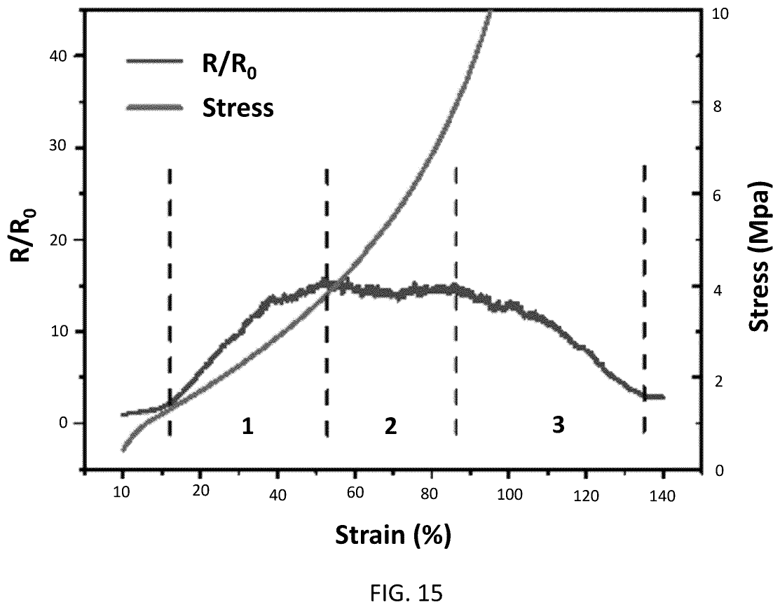

Fabrication of Knitted Sensor: The strain sensors embedded within the collar are designed to provide accurate and reliable resistance data for the detection of body motions related to swallowing events. The wearable collar features three strain sensors, strategically placed to capture the movements of the larynx and minimize interference from other bodily movements. The middle sensor is positioned directly on the larynx, the top sensor above the larynx but below the chin, and the bottom sensor below the larynx. These placements allow for the accurate detection of swallowing events while mitigating the influence of head and body movements. Made from silver-coated nylon filament yarn (40 dtex), the sensors are sensitive to variations in tension resulting from laryngeal movement. These strain sensors exhibit high sensitivity and rapid response times, enabling real-time monitoring of swallowing events. The sensitivity and response times of this type of knitted sensors in general have been reported at 60% for uniaxial tension in the 2D fabric surface and 120% for all-direction tension in the 3D fabric surface, with response times of 400 ms and 350 ms, respectively (FIG. 15) (Li et al., 2021).

The fabrication process of the wearable collar entails the use of a specialized knitting technique to seamlessly integrate the strain sensors into the fabric. The process takes place on a single circular knitting machine with a 28E machine gauge, ensuring precise and consistent sensor placement. The base structure, formed by a textured nylon filament yarn (75 dtex, 36f) and a nylon-covered spandex yarn (50 dtex), provides the foundation for the sensor integration. The silver-coated nylon filament yarn (40 dtex) is then knitted into the base fabric to form the strain sensing areas (Li et al., 2021).

After the knitting process, the collar undergoes a cut-and-sew process to shape it into a wearable form. Metal snap buttons are then attached to the leads at the ends of the strain sensors, providing a convenient and secure means of connecting the collar to the hardware, such as the mini microcontroller unit (MCU) (Li et al., 2021).

Integration of Hardware and Software Components: The integration process begins with the attachment of the hardware components to the wearable fabric collar. The metal snap buttons on the ends of the sensor leads facilitate the electrical resistance measurement as clamping the button togethers conjoin the hardware to the fabric sensor system. The knitted sensors are integrated into a voltage divider circuit, resulting in different voltage values corresponding to the changes in strain experienced during swallowing. This allows for measurements of the sensor's behavior during the swallowing process.

The MCU, Bluetooth module, and power supply are mounted on a PCB board and enclosed in a compact, lightweight housing. This housing is attached to the collar, ensuring that the system is unobtrusive and comfortable for the patient during the swallowing assessment.

In the context of software integration, the Arduino Nano is programmed using the Arduino IDE to acquire, process, and transmit the data from the strain sensors to the user interface. As described in the “Software Components” sub-section, the Arduino IDE code is designed to read the analog inputs from the three strain sensors and convert them into digital values. These values are then transmitted wirelessly via the HC-06 Bluetooth module to a customized GUI on a computer for real-time visualization and control.

The MATLAB-based GUI, as mentioned in the “Software Components” sub-section, plays a crucial role in displaying the data received from the Arduino Nano through the Bluetooth module. The GUI enables users to monitor the real-time data from the sensors, control the testing process, and save the measured data for further analysis.

The seamless integration of hardware and software components is crucial to the success of this wearable sensing device. The hardware components are carefully designed to work together with the fabric collar, while the software components ensure efficient data acquisition, processing, and display. The result is a cohesive system that enables accurate, real-time monitoring of swallowing activities, ultimately improving the diagnosis and management of dysphagia in patients (FIG. 5A).

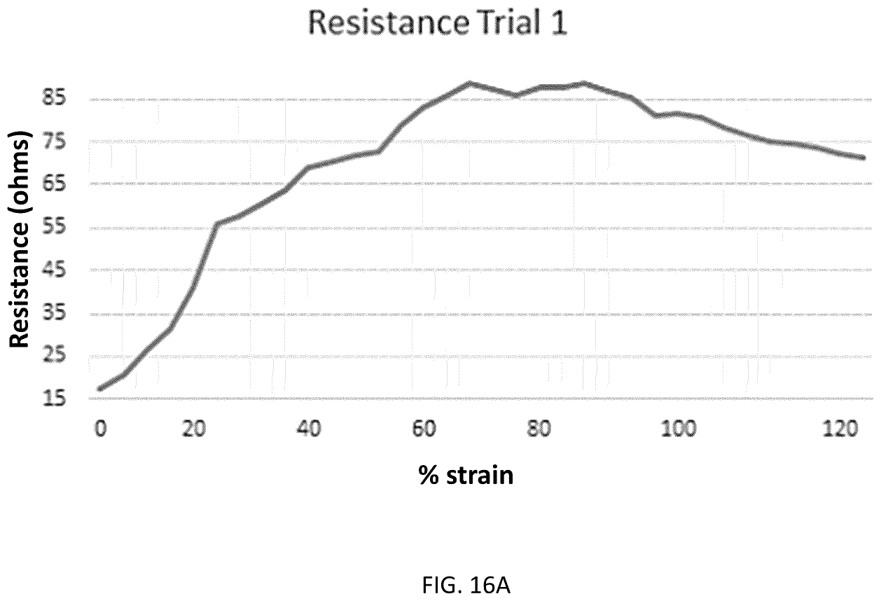

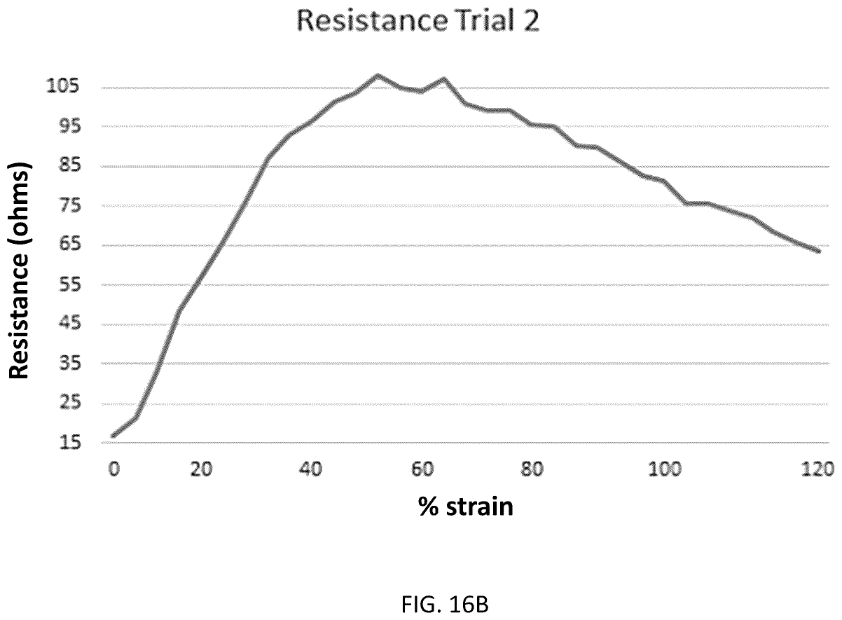

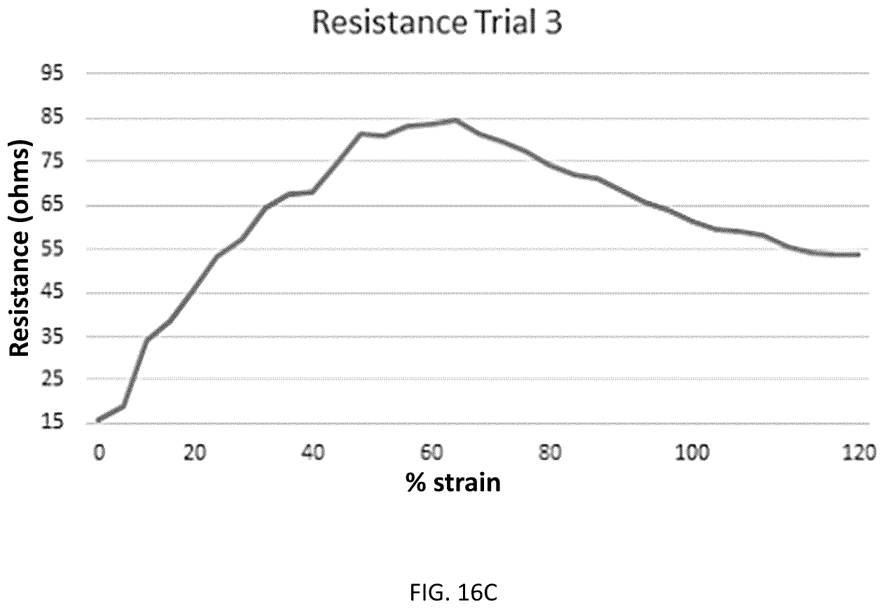

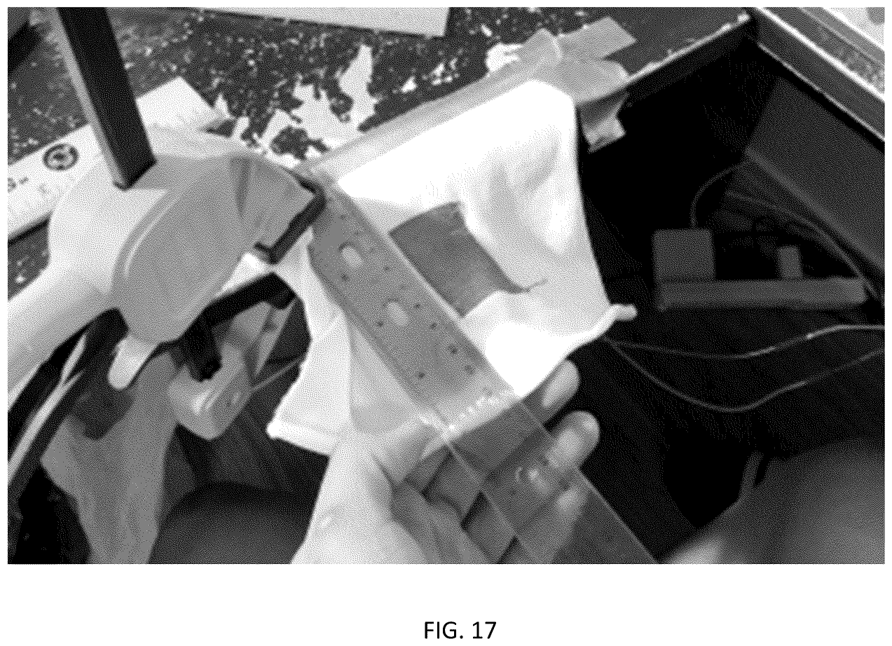

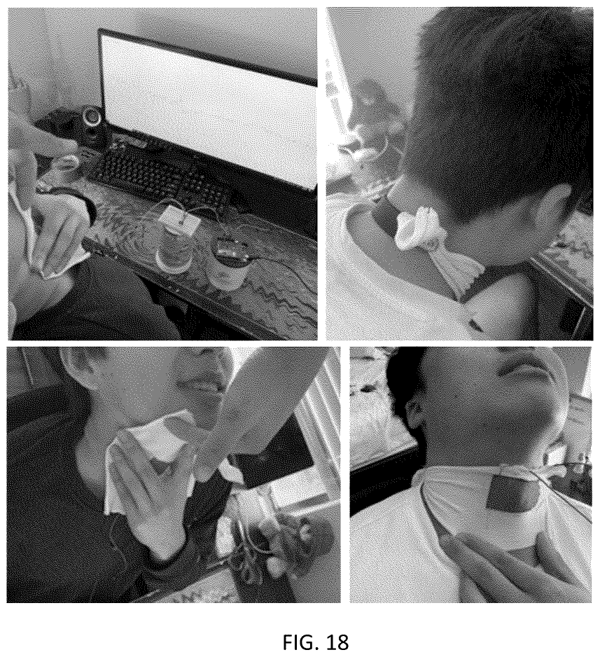

Calibration and Validation: A systematic strain calibration test is performed on the knitted fabric sensors to establish their response to different levels of strain. This process involves subjecting the sensors to varying strains ranging from 0 to 120% (FIG. 16A, FIG. 16B, FIG. 16C). The sensor's resistance is measured and recorded at each step of the calibration test, and the corresponding strain levels are correlated with the changes in electrical resistance (Huang et al., 2017).

In order to study the sensing properties of the knitted strain sensor, the sensor was stretched in the longitudinal direction in a uniaxial tensile test. The sensor was mounted on one end with a C-Clamp and duct tape (FIG. 17). A ruler is clamped on with the mounted sensor to provide a length gauge, so that it could allow the sensor to accurately stretch up to a 120% strain. For data acquisition, an Arduino code is generated with a voltage divider circuit to collect voltage and resistance changes related to the knitted strain sensor. In attempts to replicate the 2D tensile test results in Li et. al. (2021), their result's resistance change is divided into three distinct stages. In the first stage, due to gradual stretching of the knitted loops along the tensile direction, loop contact points in the strain sensor are reduced and the stress and relative resistance increase accordingly. In the second stage, the resistance increases flattens, because contact points among loops tend to increase with a larger stretching. Finally, during the third stage, the knitted loops are further straightened by stretching and the resistance shows a reversed proportional relation with the increase of tensile stress.

During the calibration process, a number of key factors are considered to ensure accurate and reliable measurements:

Consistency: The calibration test is repeated multiple times to confirm the consistency of the sensor's response to different strain levels. This helps in identifying any potential sources of error or variability in the sensor's performance (Yamada et al., 2011).

Hysteresis: Hysteresis, which refers to the difference in sensor response when subjected to increasing and decreasing strains, is evaluated during the calibration process. This allows for the identification and correction of any discrepancies between the sensor's performance during stretching and relaxation (Wang et al., 2020).

Nonlinearity: The relationship between the applied strain and the change in electrical resistance is examined to determine the linearity of the sensor's response. Any deviations from linearity can be accounted for by implementing appropriate mathematical models or compensation techniques in the data processing stage (Amjadi et al., 2014). As shown in FIG. 16A, FIG. 16B and FIG. 16C, the disclosed 2D tensile deformation model is only suitable for strain sensing within 10-60% in order for the sensor to maintain linear responsiveness.

Sensitivity: The sensitivity of the sensors is assessed by calculating the gauge factor, which is the ratio of the relative change in electrical resistance to the applied strain. A higher gauge factor indicates a higher sensitivity of the sensor to strain variations, which is essential for detecting subtle changes in the swallowing process (Li et al., 2019).

Once the calibration process is complete, the obtained calibration curve is used to convert the sensor's resistance readings into accurate strain measurements during the assessment of swallowing activities. By carefully calibrating the knitted strain sensors, the wearable fabric collar can provide reliable and precise data to aid in the accurately detecting and monitoring swallowing.

The validation process of the wearable sensing device involves a comprehensive assessment of the device's performance, including its sensitivity, specificity, and repeatability, by comparing it to existing techniques or devices used for swallow monitoring. This section will provide an overview of the steps taken to validate the developed wearable sensing device.

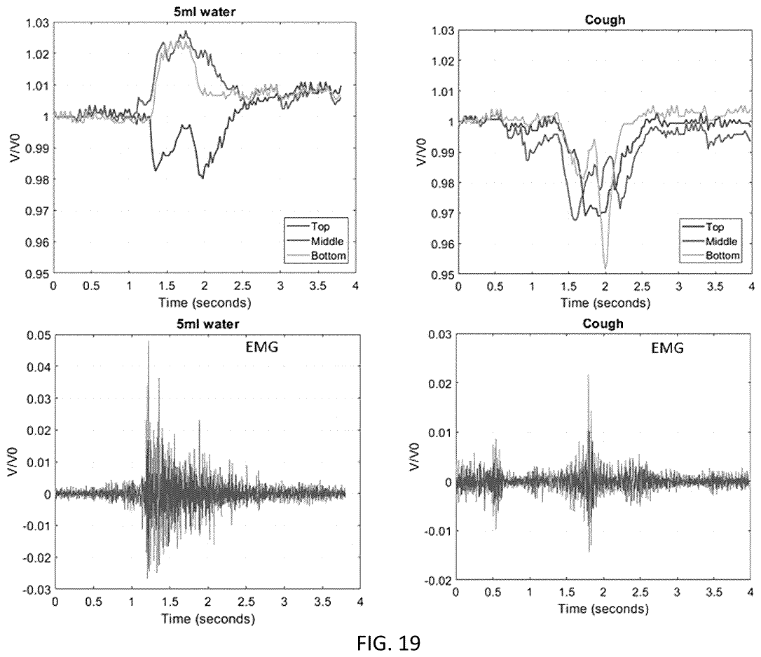

Bench Testing: Prior to subject testing, the wearable device's sensitivity and specificity were evaluated through a series of bench tests (FIG. 19). These tests were conducted using two test subjects to validate the device's response to different swallowing and non-swallowing patterns. The device's ability to detect swallows and differentiate them from other activities, such as speaking, coughing, and breathing, was assessed.

Subject Testing: After the initial bench testing, the wearable sensing device was tested on human subjects. A diverse group of healthy volunteers was recruited, and each participant was asked to perform a series of swallowing tasks, including dry swallows, saliva swallows, and swallows with varying volumes and consistencies of food and liquid. The device's performance was compared to a gold standard swallowing assessment technique, surface electromyography (sEMG), to assess its sensitivity and specificity in detecting and characterizing swallows (FIG. 19).

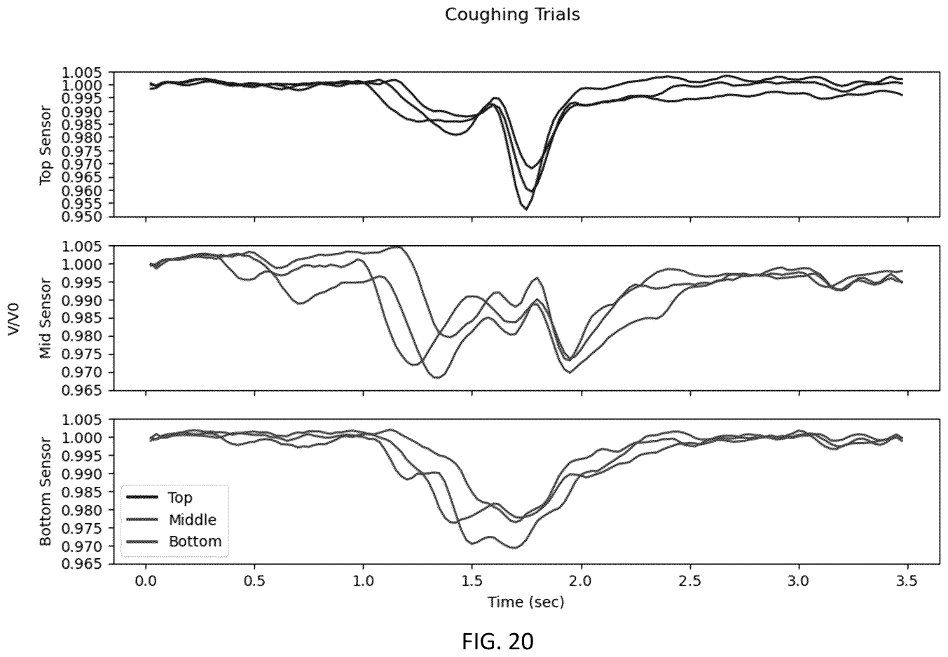

Repeatability Assessment: To evaluate the repeatability of the wearable sensing device, multiple measurements were taken for each participant during the subject testing phase. The consistency of the measurements was analyzed to ensure that the device provides reliable data under varying conditions and repeated applications (FIG. 20).

Data Analysis: The collected data from the bench and subject tests were analyzed using statistical methods, such as sensitivity, specificity, positive predictive value, negative predictive value, and intra-class correlation coefficients. These metrics allowed for a comprehensive evaluation of the wearable device's performance and accuracy in detecting and characterizing swallowing events compared to the gold standard assessment techniques.

Refinement and Optimization: Based on the validation results, the wearable sensing device are refined and optimized to improve its future performance. Any identified limitations or issues are addressed, and the device retested to ensure its enhanced performance and reliability.

Wireless Communication and Data Transmission: Disclosed herein is a detailed description of the Bluetooth protocol, its advantages, and its utilization with the MCU to enable wireless communication and data transmission for real-time monitoring and classification of swallowing performance.

This section provides a detailed description of the Bluetooth protocol, its advantages, and its utilization with the MCU to enable wireless communication and data transmission for real-time monitoring and classification of swallowing performance.

Bluetooth Protocol: In the wearable fabric collar system, the Bluetooth module used is HiLetgo HC-06, which is a class 2 device with a range of approximately 10 meters. The Bluetooth protocol relies on a master-slave architecture, where the wearable collar functions as a slave device that communicates with a master device, such as a smartphone or computer.

Advantages: The utilization of Bluetooth technology in the wearable fabric collar system offers several advantages, such as low power consumption, ease of use, and real-time data transmission. The low power consumption of the HC-06 module allows for extended use of the device without frequent battery replacement, making it suitable for continuous monitoring of swallowing performance in patients with dysphagia. Furthermore, the Bluetooth protocol allows for seamless integration with a wide range of devices, enabling the development of user-friendly applications for data visualization and analysis.

Integration with the MCU: The integration of the Bluetooth module with the Elegoo Nano Board (based on the ATmega328P) involves connecting the module's RX and TX pins to the MCU's TX and RX pins, respectively. As the module operates at 3.3V, a voltage divider is used to step down the MCU's 5V output to a safe 3.3V level, preventing damage to the module. The Arduino Nano then uses the Bluetooth module to transmit sensor data wirelessly to a master device, such as a computer or smartphone, for real-time monitoring and assessment.

Software Configuration: To facilitate wireless communication between the wearable fabric collar and the master device, a custom graphical user interface (GUI) is developed using MATLAB's App Designer. This GUI enables the user to control the testing process, visualize the data in real-time, and save the recorded measurements for further analysis.

Disclosed herein are the details of data transmission, including the sampling rate and baud rate, as well as the data processing techniques used, such as filtering and noise reduction, to ensure the accuracy and reliability of the acquired data.

Sampling Rate and Baud Rate: In this study, a sampling rate of 40 Hz is employed, which provides adequate resolution to capture the dynamics of swallowing events. To transmit the data from the Elegoo Nano Board (based on the ATmega328P) to the master device, a baud rate of 9600 is used. This baud rate facilitates the efficient and reliable transmission of the sampled data over the Bluetooth connection.

Filtering: The acquired data may contain unwanted noise and artifacts that can affect the accuracy of the swallowing assessment. To mitigate this issue, the Savitzky-Golay filtering technique is applied to the data (Savitzky & Golay, 1964). This filter smooths the data while preserving its essential features by fitting a low-degree polynomial to a sliding window of data points. In this study, a window size of 21 samples and a polynomial order of 9 are used, providing an effective trade-off between noise reduction and signal preservation.

Noise Reduction: In addition to filtering, further noise reduction is achieved by isolating the swallowing and non-swallowing events in the recorded data.

This is done by manually examining the output CSV files generated by the MATLAB GUI and aligning the trials for each action and subject. This procedure eliminates extraneous noise and retains only the relevant signal components. Moreover, a third sensor is placed below the middle sensor (larynx) to serve as a reference for eliminating head and body movement artifacts, further improving the signal quality.

Data Processing Workflow: The overall data processing workflow begins with the acquisition of sensor data at a 40 Hz sampling rate. The raw data is transmitted at a baud rate of 9600 via Bluetooth to the master device, where it is visualized and stored using the custom MATLAB GUI. The data is then filtered using the Savitzky-Golay technique and subjected to a manual segmentation process to isolate and categorize the swallowing and non-swallowing events. The processed data can then be used for further analysis and interpretation, providing valuable insights into the swallowing performance of patients with dysphagia.

Subject Testing and Data Collection: Disclosed herein is the subject recruitment process, the inclusion and exclusion criteria, and the demographics of the study population for the wearable fabric collar system's validation and performance assessment were discussed to aim to recruit a diverse cohort of participants, ensuring a representative sample for the evaluation of swallowing performance.

Participant Recruitment: The study involved a total of 12 participants, including an equal amount of male and female participants. The recruitment process was conducted using multiple channels, such as email invitations, word of mouth, and announcements made by teachers or professors during class sessions.

Inclusion and Exclusion Criteria: To ensure a consistent and reliable evaluation of the wearable fabric collar system, specific inclusion and exclusion criteria were established. The 6 males and 6 females were aged between 18 and 33, with a requirement of under the age 45. All participants were required to be healthy, with no history of swallowing disorders or any medical condition that could potentially affect the swallowing process. This criterion helped to obtain baseline data on healthy swallowing performance, which can be used as a reference for future studies involving patients with dysphagia.

Demographics: The study population consisted of a diverse group of individuals, including students and full-time workers, in order to capture the variability in the swallowing performance among different lifestyles and daily activities. The age range of 18-33 was selected to focus on a young adult population that could provide valuable insights into normal swallowing physiology, serving as a foundation for future research targeting older adults or individuals with specific health conditions.

Ethical Considerations: The participants were informed about the purpose of the study, the procedure, and their rights as study subjects, including the right to withdraw from the study at any time without any consequences. Informed consent was obtained from all participants before their involvement in the trials.

Experimental Setup and Procedure for Testing: Disclosed herein is the detailed setup and procedure employed during the participant testing process, including the positioning of the collar, the specific swallowing tasks performed, and the simultaneous electromyography (EMG) measurement.

Experimental Setup: The participants were seated upright while wearing the wearable collar, equipped with three strain sensors, and EMG electrodes. The collar was carefully placed around the neck, ensuring that the laryngeal prominence was positioned directly on the middle sensor. The other two sensors were positioned above and below the laryngeal prominence to accommodate for movements associated with swallowing tasks. In parallel, EMG electrodes were taped below the chin bilaterally 1 cm. from midline and on the forehead as a ground (green), enabling the concurrent measurement of muscle activity during swallowing and non-swallowing tasks (Crary et al., 2005; Ertekin & Aydogdu, 2003).

Testing Procedure: The following step-by-step procedure was followed for the participant testing:
a. Before the clinical testing, a pre-test survey is filled out by the participants to assess their overall health and physical ability, focusing on their swallowing function.
b. Ensure the hardware and data acquisition program were set up and ready to go.
c. Fit the collar and EMG electrodes onto the participant, ensuring the proper positioning of the sensors.
d. Start the data acquisition program and instruct the participant to remain still for approximately 3 seconds to account for any initial lag.
e. Have the participant perform one of the 17 specified tasks, with a list of tasks provided in the description below.

TABLE 4
Clinical Trial Task List
Tasks List

| | |
|---|---|
| Swallowing Actions | Non-Swallowing Actions |
| 20 ml water | Chin Tuck |
| 5 ml water | Cough |
| Saliva Swallow | Head Rotation to the Left |
| Sip Water Through Straw | Inhale and Exhale |
| Sip Water with Effortful | Say ‘Ah’ |
| Swallow | |

f. Once the task was completed, record data for another 3 seconds.
g. Stop the data acquisition program and save the collected data as a CSV file.
h. Repeat the process for each of the 17 tasks, performing three trials for each action until all data have been collected. One of the tasks, reading a paragraph, had two trials; however, each trial consisted of different written content for the patients to read.
i. After the data collection is completed, the participant is asked to complete a comfortability survey based on a numbered rating scale.

Rationale for EMG Electrode Placement: EMG electrodes were placed below the chin to measure the activity of the submental muscles, primarily a group of suprahyoid muscles that contribute to the complex swallowing process (Crary et al., 2005). The suprahyoid muscles play an essential role in the swallowing process. During the pharyngeal phase of swallowing, these muscles contract to elevate the hyoid bone and the larynx. This movement assists in the opening of the upper esophageal sphincter, allowing the food bolus to pass from the pharynx into the esophagus. Simultaneously, it helps close the epiglottis, a flap of cartilage located at the base of the tongue, to prevent aspiration of the food or liquid into the airways. By measuring the electrical activity of these muscles, it is possible to differentiate between swallowing and non-swallowing events, providing valuable information for the validation of the wearable collar system.

Parameters included: The primary parameters included during the data collection process were age, sex, and health status of the participants. Prior to the experiment, a pre-test survey was conducted to assess the participants' overall health and physical ability, particularly focusing on their swallowing function. This survey helped to ensure that only healthy individuals with no history of swallowing disorders were included in the study. Additionally, after the experiment, participants were asked to complete a comfortability survey to evaluate their experience with the wearable collar and provide valuable feedback for potential design improvements. According to Table 5, the low average scores of both the pre-test and post-test indicated that the included participants didn't struggle with performing swallowing or non-swallowing actions and found that the disclosed wearable device was relatively comfortable.

TABLE 5
Pre- and Post-Test Survey Scale

| | | Post-Test | |
|---|---|---|---|
| Pre-Test Survey | | Comfortability Survey | |
| 0 | 100 | 0 | 100 |
| Never | Occurs | No | Worse |
| occurs/No | every | discomfort | discomfort |
| difficulty | time/Unable | at all | imaginable |
| | to perform | | |

Average score

| | |
|---|---|
| 4.058 | 7.333 |

Duration of Data Collection: Each participant completed 10 tasks (swallowing and non-swallowing) for three trials each, as outlined in the “Experimental Setup and Procedure for Participant Testing” sub-section. The total data collection time for each participant, therefore, was the time taken to complete these 30 actions (10 tasks×3 trials), plus the time required for sensor calibration and resting periods between trials. The exact duration of data collection for each participant varied depending on individual performance and adherence to the testing procedure. Nevertheless, the duration was kept reasonably consistent across all subjects to ensure comparability of the data.

Storage and Organization of Data: The data collected during the experiments were stored as CSV files, with each file corresponding to a specific trial. To facilitate easy access and organization, the files were stored in separate folders for each participant, identified by their unique subject number. This storage and organization system facilitated efficient data retrieval, allowing for convenient access and analysis during the subsequent data processing and analysis phase of the study.

Data Analysis and Processing: Disclosed herein is a detailed description of the preprocessing steps undertaken to clean the raw data obtained from the experiments, ensuring that the data is suitable for further analysis and interpretation. Preprocessing is a crucial step in the data analysis pipeline, as it helps eliminate noise and artifacts, thereby enhancing the accuracy of the results derived from the processed data.

Noise Reduction: One of the first preprocessing steps involved isolating individual swallowing and non-swallowing events by aligning the trials for each subject. This was achieved by examining the output CSV files generated by the data acquisition program and aligning each action's trials for every participant. By focusing solely on the action signals, extraneous noise from the data was effectively eliminated. Additionally, a third sensor was placed below the middle sensor (near the larynx) tasked with both monitoring and eliminating potential interference from head and body movements. This strategic sensor placement ensures that the data extracted from the middle and top sensors primarily represents the targeted swallowing/non-swallowing tasks.

Savitzky-Golay Filtering: To further enhance the quality of the collected data, a Savitzky-Golay filter was applied to the strain sensor signals. This filtering technique smooths the data by fitting successive sub-sets of adjacent data points with a low-degree polynomial using the method of least squares (Savitzky & Golay, 1964). For this study, a window size of 21 data points and a polynomial order of 9 were employed. The Savitzky-Golay filter is particularly suitable for this application as it preserves the high-frequency content of the data, ensuring that the fine details of the strain signals, critical for differentiating between swallowing and non-swallowing events, are retained.

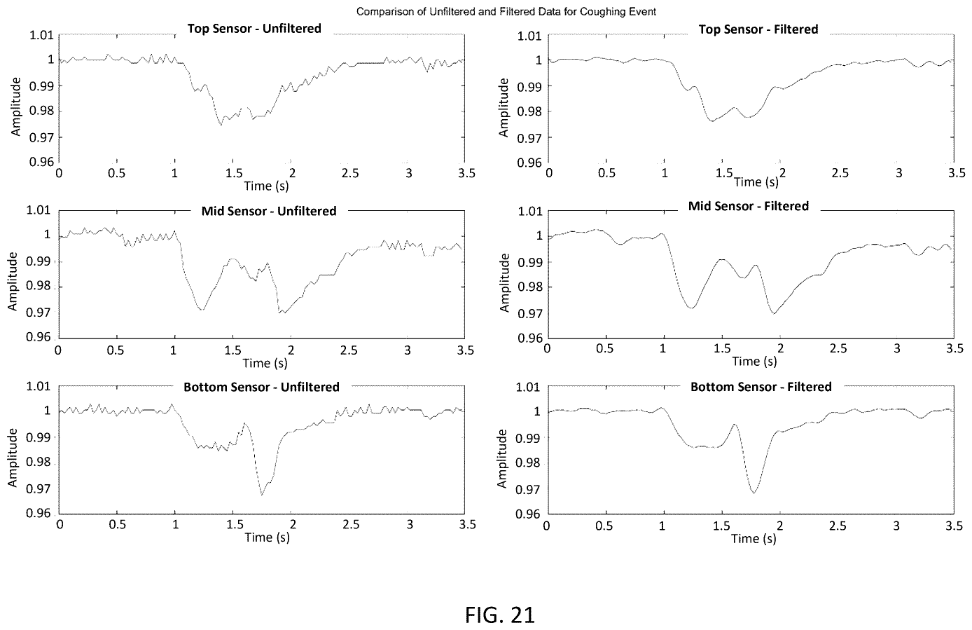

The filtering process plays a vital role in enhancing the signal quality and removing undesired noise from the raw sensor data obtained from the wearable sensing device (FIG. 21). The implemented filtering algorithm is the Savitzky-Golay filter, which combines local polynomial regression fitting and moving average techniques (Savitzky & Golay, 1964). The filter parameters, window size (21) and polynomial order (9), were determined empirically to optimize the balance between smoothing and preserving relevant signal characteristics.

The window size specifies the number of points within the moving window, while the polynomial order dictates the degree of the fitted polynomial. The choice of Savitzky-Golay filtering is motivated by its ability to preserve higher moments in the data and its effectiveness in reducing high-frequency noise while retaining essential features of the underlying signal (Savitzky & Golay, 1964).

Comparing the raw and filtered sensor data, the latter exhibits a more refined signal with less high-frequency noise, enabling a clearer representation of swallowing patterns. The unfiltered data contains various undesired disturbances, which could hinder the accurate identification of critical features or thresholds for dysphagia assessment. In contrast, the filtered data highlights the essential dynamics of swallowing events, facilitating the extraction of relevant features for further analysis. Raw and filtered sensor data for each task performed by the subjects are included in the Appendix.

Data Segmentation: Once the raw data was filtered and smoothed, it was segmented into individual trials corresponding to the 10 tasks performed by each participant. To enhance the precision of this segmentation, each participant was instructed to remain static for 3 seconds prior to and following each task. This provision of ‘quiet’ periods ensured a clear demarcation between the trials, allowing for the precise identification of the start and end times of each task. This step allows for the isolation of specific actions and facilitating the subsequent analysis and comparison of the data across different trials and participants.

In the context of swallowing assessment using the wearable sensing device, extracting relevant features from the collected data is essential for understanding and interpreting the swallowing and non-swallowing actions of patients. The features extracted from the raw data can reveal essential information about the swallow duration, frequency, and amplitude, and help differentiate between normal and dysphagic swallowing patterns. This sub-section delves into the feature extraction methods employed in this study and their mathematical foundations.

Standard Deviation: In the context of swallowing assessment, a higher standard deviation may indicate greater variability in swallowing pressure or muscle activity during the swallowing process. Standard deviation can help identify potential abnormalities or inconsistencies in swallowing patterns.

Dip Amplitude: Dip amplitude refers to the magnitude of the negative peak in a bio-signal wave. It represents the difference between the baseline value and the minimum value of the wave. Mathematically, the dip amplitude can be calculated using the following equation:

$$Dip Ampltiude = Baseline Value - Minimum Value \qquad Equation 2$$

The baseline value is typically the static signal value when no significant activity is occurring, while the minimum value is the lowest point of the wave.

The dip amplitude helps to quantify the magnitude of a negative peak in a wave, which can be useful for characterizing certain aspects of swallowing. In some cases, the dip amplitude can be indicative of the strength or intensity of the physiological event represented by the wave. For example, larger dip amplitudes might be associated with stronger or more pronounced physiological events, while smaller dip amplitudes might indicate weaker or less significant events. Analyzing the dip amplitude can provide insights into the swallowing strength and coordination of the patient.

Time Duration: Time duration refers to the time interval between the start and end of a swallow. The action of swallowing inhibits certain slope thresholds when it starts and finishes. This feature is essential in understanding the speed and efficiency of the swallowing process. Abnormal swallowing durations may indicate neuromuscular issues relating to dysphagia or other swallowing disorders (Perlman, Palmer, McCulloch, & Vandaele, 1999).

To mathematically determine the time duration of a biosignal wave, first compute the slope between five data points (Δy/Δx). Δx being time change and Δy representing the biosignal output. Given a set of data points (xᵢ,yᵢ) for i=1, 2, I, N, the slope at any point I can be calculated as:

$${s}_{i} = \frac{( {y}_{i + 2} - {y}_{i - 2} )}{( {x}_{i + 2} - {x}_{i - 2} )} \qquad Equation 3$$

This computation using five data points helps in reducing the noise impact by taking an average slope over a broader interval.

The start threshold (Tₛₜₐᵣₜ) is set at a slope greater than 0.04:

$${T}_{start} : {s}_{i} > 0.04 \qquad Equation 4$$

The time duration continues until the stop threshold (Tₛₜₒₚ) is met:

$${T}_{start} : {s}_{i} < 0.01 \qquad Equation 5$$

The region between the start and stop thresholds represents the time duration of interest. Denote the start time as tₛₜₐᵣₜ and the end time as t_{end}:
tₛₜₐᵣₜ: The first-time instance where the slope sᵢ>0.04
t_{end}: The first-time instance after tₛₜₐᵣₜ where the slope sᵢ<0.01

The time duration (TD) can be computed as:

$$TD = {t}_{end} - {t}_{start} \qquad Equation 6$$

Area Under the Curve (AUC): The area under the curve (AUC) of a biosignal wave is a quantitative measure of the total “activity” represented by the wave. It can be computed using various numerical integration techniques, one of which is the trapezoidal rule. The trapezoidal rule approximates the integral of a curve by summing the areas of a series of trapezoids under the curve. Disclosed herein is an overview of the trapezoidal rule for calculating the AUC: The AUC represents the integral of the data over the duration of a swallow.
1. Divide the curve into n equally spaced segments along the x-axis, where each segment has a width Δx. The endpoints of the segments are x₀, x₁ . . . xₙ.
2. Calculate the height of the curve at each endpoint (y=f(xᵢ), where f(xᵢ) is the value of the biosignal wave at the xᵢ position).
3. Calculate the area of each trapezoid under the curve:

$${Area}_{i} = ( \frac{1}{2} ) * ( {y}_{i} + {y}_{i + 1} ) * Δ x \qquad Equation 7$$

where yᵢ and yᵢ₊₁ are the heights of the curve at the endpoints of the i-th segment.
4. Sum the areas of all trapezoids to compute the AUC:

$$AUC = \sum {Area}_{i} for i = 0 , 1 , … , n - 1 \qquad Equation 8$$

This feature can provide insights into the overall energy or effort exerted during the swallowing process and may help identify abnormalities in muscle coordination or strength.

Mean Crossing Rate: Mean crossing rate (MCR) is a measure of the number of times a wave crosses its mean value within a given period. It provides information about the oscillatory nature and frequency content of the signal. Disclosed herein is a mathematical overview of calculating the mean crossing rate for a given biosignal wave:
1. Calculate the mean_value(μ) of the biosignal wave.
2. Identify the mean crossing points. Here's a breakdown of the MATLAB code:
   I. swallow_data>mean_value: This creates a logical array where each element is ‘1’ (true) if the corresponding element in trial_data is greater than mean_value, and ‘0’ (false) otherwise.
   II. diff(swallow_data>mean_value): This calculates the difference between adjacent elements in the logical array. A positive difference (equal to 1) indicates a crossing from below the mean to above the mean, while a negative difference (equal to −1) indicates a crossing from above the mean to below the mean.
   III. sum(diff(swallow_data>mean_value)==1): This sums up the number of positive differences (equal to 1), which represent the mean crossings from below to above the mean. This count represents the total number of mean crossings in the signal.

This feature can reveal the periodicity or regularity of the swallowing signal, with higher mean crossing rates indicating increased variability or irregularity in the swallowing process.

Skewness: Skewness is a statistical measure that describes the asymmetry of a distribution. In the context of a biosignal wave, skewness can provide insights into the shape and symmetry of the underlying distribution of the signal's amplitude values. Disclosed herein is a mathematical overview of calculating skewness for a given dataset:
1. Calculate the mean (μ) of the biosignal wave.
2. Calculate the standard deviation (σ) of the biosignal wave.
3. Compute the cube of the deviations from the mean:

$${( {y}_{i} - μ )}^{3} \qquad Equation 9$$

4. Calculate the mean of the cubed deviations:

$$E ( {( {y}_{i} - μ )}^{3} ) = \frac{\sum {( {y}_{i} - μ )}^{3}}{N} \qquad Equation 10$$

5. Calculate the skewness (y) of the dataset:

$$γ = \frac{E ( {( {y}_{i} - μ )}^{3} )}{{σ}^{3}} \qquad Equation 11$$

Skewness is a measure of the asymmetry of a distribution. For a biosignal wave, skewness can provide insights into the shape and symmetry of the underlying distribution of the signal's amplitude values. A positive skewness indicates that the distribution has a longer tail on the right side, while a negative skewness indicates a longer tail on the left side. A skewness value close to 0 suggests that the distribution is relatively symmetric.

Kurtosis: Kurtosis is a statistical measure that describes the “tailedness” or the shape of the probability distribution of a dataset. In the context of a biosignal wave, kurtosis provides insights into the shape, specifically the peakedness or flatness of the distribution of the signal's amplitude values. Disclosed herein is a mathematical overview of calculating kurtosis for a given dataset:
1. Compute the fourth power of the deviations from the mean:

$${( {y}_{i} - μ )}^{4} \qquad Equation 12$$

2. Calculate the mean of the fourth power deviations:

$$E ( {( {y}_{i} - μ )}^{4} ) = \frac{( \sum {( {y}_{i} - μ )}^{4} )}{N} \qquad Equation 13$$

where Σ is the sum of the data points, and N is the total number of data points.
3. Calculate the kurtosis (k) of the dataset:

$$k = \frac{E ( {( {y}_{i} - μ )}^{4} )}{{σ}^{4}} \qquad Equation 14$$

where σ is the standard deviation.

A higher kurtosis value indicates a more peaked distribution with heavier tails, while a lower kurtosis value suggests a flatter distribution with thinner tails. A kurtosis value of 3 indicates a normal distribution (mesokurtic). If the kurtosis is greater than 3, the distribution is considered leptokurtic (heavy-tailed), and if it's less than 3, the distribution is platykurtic (light-tailed).

Prominence: Prominence is a measure used in the context of biosignal waves to quantify the relative height and importance of a peak within a signal. It is calculated by comparing the peak's height to its neighboring peaks and the signal's baseline. Prominence is determined by the minimum vertical distance that one must descend from the peak to a lower elevation point on either side before climbing to a higher peak. Disclosed herein is a mathematical overview of calculating prominence for a given biosignal wave:
1. Identify the peak of interest pk in the signal, along with its amplitude (Ampₚ).
2. Find the neighboring peaks on either side of the pk. These are the peaks closest to peak that have a higher amplitude than pk.
3. Determine the highest baseline points on either side of the pk, within the intervals defined by the neighboring peaks. The baseline points are the points with the lowest amplitude in the interval that separates pk from its neighboring peaks. Let the amplitudes of these baseline points be Amp_{bl1} and Amp_{bl2}.
4. Calculate the prominence of the peak pk:

$$Prom = {Amp}_{p} - max ( {Amp}_{bl 1} , {Amp}_{bl 2} ) \qquad Equation 15$$

Prominence is a measure of the relative importance of a peak in a biosignal wave. It considers the peak's height compared to its neighboring peaks and the signal's baseline. A higher prominence value indicates that the peak is more significant or distinct within the signal, while a lower prominence value suggests that the peak is less prominent or less important. Prominence can be useful for identifying and characterizing specific features within a biosignal wave and comparing different peaks within a single signal or between multiple signals.

Slope: The slope of a biosignal wave is a measure of the rate of change in the signal's amplitude with respect to time. It quantifies the steepness or inclination of the wave at a particular point or over a specified interval. Mathematically, the slope of a continuous function is given by the first derivative with respect to time. Disclosed herein is an overview of calculating the slope of a biosignal wave:
1. Let y(t) represent the biosignal wave, where y is the amplitude and t is time.
2. Calculate the first derivative of y(t) with respect to time (t), which represents the instantaneous slope of the wave at any given time:

$$\frac{dy}{dt} = {y}^{′} ( t ) \qquad Equation 16$$

For a discrete biosignal, the slope can be approximated by computing the difference between successive amplitude values and dividing by the time interval between those values (Δt):
3. Given a sequence of amplitude values yᵢ at time points tᵢ, calculate the approximate slope at each point i:

$${Slope}_{i} \approx \frac{( {y}_{( i + 1 )} - {y}_{i} )}{( {t}_{( i + 1 )} - {t}_{i} )} \qquad Equation 17$$

The slope of a biosignal wave quantifies the rate of change in the signal's amplitude with respect to time. It can provide insights into the signal's dynamics, such as the rapidity of transitions between different physiological states or the presence of specific events, like abrupt changes in amplitude. It is worth noting that calculating the slope directly from raw biosignal data may be sensitive to noise; in practice, it's often useful to apply some form of smoothing or filtering to the data before calculating the slope.

Wavelet Entropy: Wavelet entropy is a measure of the complexity or randomness of a signal in the context of wavelet analysis. Wavelet entropy is computed by first decomposing the signal using a wavelet transform, which results in a multi-scale representation of the signal's frequency content. The wavelet entropy is then calculated based on the distribution of energy across the different scales of the wavelet decomposition. Disclosed herein is a mathematical overview of calculating the wavelet entropy for a biosignal wave:
1. Compute the wavelet transform of the biosignal. For a discrete signal x[n], the wavelet transform coefficients are given by:

$${W}_{x} ( a , b ) = \sum x [ n ] * ψ ( \frac{( n - b )}{a} \qquad Equation 18$$

where ψ is the wavelet function, a represents the scale, b represents the translation, and Σ is the sum over all values of n.
2. Calculate the wavelet energy (E) for each subband (level) of the wavelet transform:

$${E}_{j} = \sum \vert {W}_{x} ( {a}_{j} , b ) {\vert}^{2} \qquad Equation 19$$

where aⱼ is the scale associated with the j-th level.
3. Calculate the total wavelet energy (E_{T}):

$${E}_{T} = \sum {E}_{j} \qquad Equation 20$$

4. Compute the normalized wavelet energy (Pⱼ) for each subband:

$${P}_{j} = \frac{{E}_{j}}{{E}_{T}} \qquad Equation 21$$

5. Calculate the Shannon wavelet entropy (H_{w}):

$${H}_{w} = - \sum {P}_{j} * log ( {P}_{j} ) \qquad Equation 22$$

Wavelet entropy is a measure of the complexity or randomness of a biosignal wave in the context of wavelet analysis. It provides insights into the distribution of energy across different scales of the wavelet decomposition. A higher wavelet entropy indicates a more complex or random signal, while a lower wavelet entropy suggests a more regular or predictable signal. The interpretation of wavelet entropy depends on the type of biosignal and the context in which it is being studied. It can be useful for characterizing the complexity of physiological signals, identifying specific features, and comparing different signals or signal segments.

Entropy Rate: The function wentropy in MATLAB computes the entropy of a signal using a specified entropy function, with ‘shannon’ being one of the available options. Entropy, in the context of information theory, is a measure of the unpredictability or randomness of a signal. In the case of the Shannon entropy, it quantifies the average amount of information needed to describe the signal. Disclosed herein is a step-by-step mathematical overview of calculating the Shannon entropy for a discrete biosignal wave:
1. Determine the probability distribution of the amplitude values in the signal. This can be achieved by dividing the number of occurrences of each unique amplitude value by the total number of data points in the signal.
2. Let p(xᵢ) represent the probability of occurrence for each unique amplitude value xᵢ.
3. Calculate the information content for each unique amplitude value xᵢ:

$$l ( {x}_{i} ) = - {log}_{2} p ( {x}_{i} ) ) \qquad Equation 23$$

4. Compute the Shannon entropy (H) of the signal by taking the weighted average of the information content of each unique amplitude value xᵢ:

$$H = \sum [ p ( {x}_{i} ) * l ( {x}_{i} ) ] = - \sum [ p ( {x}_{i} ) * {log}_{2} ( p ( {x}_{i} ) ) ] \qquad Equation 24$$

The entropy rate, or Shannon entropy, is a measure of the unpredictability or randomness of a biosignal wave. It quantifies the average amount of information required to describe the signal. A higher entropy rate indicates a more complex or random signal, while a lower entropy rate suggests a more regular or predictable signal. The interpretation of entropy rate depends on the type of biosignal and the context in which it is being studied. It can be useful for analyzing the complexity of physiological signals and comparing different signals or signal segments.

Bandwidth: The bandwidth of a biosignal wave is calculated using a power spectral density (PSD) estimation, which is obtained using the periodogram method. The bandwidth represents the range of frequencies within which a significant portion of the signal's power is contained. It provides insights into the spread or dispersion of the frequency content of the signal. Here's a mathematical overview of calculating the bandwidth for a biosignal wave:
1. Calculate the periodogram of the trial_data to obtain the power spectral density (PSD) estimation. The periodogram is computed using the squared magnitude of the discrete Fourier transform (DFT) of the signal divided by the number of samples (N):

$$Pxx ( f ) = ( 1 / N ) * {\vert DFT ( trial\_data ) \vert}^{2} \qquad Equation 25$$

2. Compute the spectral centroid of the PSD:

$${Spectral}_{Centroid} = \frac{\sum ( {F}_{i} * {Pxx}_{i} )}{\sum ( {Pxx}_{i} )} \qquad Equation 26$$

Here, Pxx represents the PSD, and F denotes the corresponding frequencies.
3. Calculate the bandwidth of the signal:

$$Bandwidth = \sqrt{\frac{\sum ( {( {F}_{i} - {Spectral}_{Centroid} )}^{2} * {Pxx}_{i} )}{\sum ( {Pxx}_{i} )}} \qquad Equation 27$$

The bandwidth of a biosignal wave quantifies the spread or dispersion of the frequency content of the signal. It provides insights into the range of frequencies within which a significant portion of the signal's power is contained. It can be useful for characterizing the spectral properties of swallowing signals and comparing different action signals or signal segments.

Spectral Centroid: The spectral centroid corresponds to the “center of mass” of the frequency distribution; refer to bandwidth equation. Analyzing these features can provide insights into the frequency content and overall energy distribution of the swallowing signal and help identify deviations from normal swallowing patterns.

In summary, the feature extraction methods employed in this study aim to capture the essential characteristics of swallowing and non-swallowing actions through a combination of time-domain, frequency-domain, and statistical features. These features were selected based on their relevance to the physiological processes involved in swallowing and their potential to differentiate between swallowing and non-swallowing patterns. By analyzing the extracted features, healthcare professionals can gain valuable insights into the swallowing process, ultimately leading to improved assessment and management of dysphagia in patients.

It is crucial to note that the choice of features should be informed by a clear understanding of the underlying physiological processes, as well as the specific objectives of the research. Moreover, the selection of features should strike a balance between capturing the relevant information and avoiding overfitting, which may lead to poor generalizability in the machine learning algorithms employed for swallowing pattern classification.

By employing the feature extraction methods described in this sub-section, this research aims to develop a comprehensive understanding of the swallowing and non-swallowing actions of patients using the wearable sensing device. The analysis of these features will enable the identification of potential abnormalities in the swallowing process and contribute to the development of more effective strategies for dysphagia assessment and management.

With a detailed and engineering-driven approach to feature extraction, the study ensures that the extracted features are not only relevant and informative but also grounded in the physiological and biomechanical aspects of swallowing. This approach ultimately contributes to the overall quality and rigor of the research.

Disclosed herein are several machine learning techniques employed to analyze, classify, and predict the swallowing patterns captured by the wearable sensing device. These techniques were implemented using MATLAB's classification learner app, which provides a user-friendly interface for training, evaluating, and comparing different machine learning models. The selected techniques include:
Decision Trees: Decision trees are a popular machine learning method used for classification and regression tasks. They work by recursively partitioning the input space and defining a set of decision rules based on the most discriminative features of the data (Quinlan, 1986). In the context of this study, decision trees were employed to classify swallowing patterns by examining the relationships between the extracted features and the underlying swallowing behavior.
Support Vector Machines (SVM): SVMs are a powerful class of supervised learning algorithms that can handle both linear and nonlinear classification problems. They aim to find the optimal hyperplane that best separates the data into different classes, with the maximum margin (Cortes & Vapnik, 1995). In this study, SVMs were utilized to classify swallowing patterns by mapping the feature space into a higher-dimensional space using kernel functions and finding the optimal decision boundary.
k-Nearest Neighbors (k-NN): k-NN is a simple and effective instance-based learning algorithm that can be employed for classification and regression tasks. It works by finding the k training instances closest to a new input and assigning the most common class label among the k nearest neighbors (Cover & Hart, 1967). In the context of this research, k-NN was used to classify swallowing patterns based on the similarity between the feature vectors of the training data and the test data.
Ensemble Methods: Ensemble methods are a family of machine learning techniques that combine multiple base models to improve classification performance. These methods work by exploiting the complementary strengths of different algorithms and reducing the likelihood of overfitting (Zhou, 2012). In this study, ensemble methods, such as Bagging, Boosting, and Random Forests, were employed to enhance the classification performance of the individual models and achieve a more robust and accurate swallowing pattern classification.
Artificial Neural Networks (ANN): ANNs are a class of machine learning models inspired by the structure and function of biological neural networks. They consist of interconnected nodes or neurons that process and transmit information through weighted connections (Jain et al., 1996). In this research, ANNs were utilized to model the complex relationships between the extracted features and the swallowing behavior, enabling the classification of various swallowing patterns.

In summary, the machine learning techniques employed in this study were selected based on their ability to handle complex and high-dimensional data, as well as their suitability for classification tasks. By leveraging the capabilities of these algorithms, the study aimed to develop accurate and robust models for analyzing, classifying, and predicting swallowing patterns.

The Chi-Squared (Chi2) algorithm works by comparing observed data with expected data to assess how well they align. In the context of feature selection, it evaluates the independence between each feature and the class labels. The underlying principle is that features should be closely related to the class label and relatively independent from each other.

Mathematically, for each feature, the Chi2 statistic is calculated using the formula:

$${X}^{2} = \sum \frac{{( {O}_{ij} - {E}_{ij} )}^{2}}{{E}_{ij}} \qquad Equation 1$$

where Oᵢⱼ represents the observed frequency and Eᵢⱼ represents the expected frequency under the assumption of independence. A high Chi2 score implies that the feature and the class label are dependent, and thus the feature is important for classification. Conversely, a low Chi2 score indicates that the feature is likely not important, as its distribution does not depend on the class label.

The Chi2 algorithm was used to determine the independence between the extracted features from knitted strain sensor data and the class labels, which were whether the subject was swallowing or not. The features that showed the most dependency (and hence, had the highest Chi2 scores) were deemed to be the most significant in differentiating between swallowing and non-swallowing actions.

These top-ranked features for each sensor, assigned an importance score based on their Chi2 statistic, were then presented and analyzed in terms of their relevance to the mechanics of the swallowing action. This application of the Chi2 algorithm not only aids in understanding the relationship between the features and the swallowing process, but also serves as an effective feature selection method for machine learning tasks, particularly for the included dataset from healthy participants.

Safety Measure: As the research involves the use of a wearable collar for monitoring swallowing and non-swallowing events, it is crucial to minimize any potential hazards, such as skin irritation, choking, or discomfort, associated with the device and the experimental procedure. To ensure participant safety, the study was conducted under controlled conditions. The wearable collar was designed with comfort and safety in mind, using materials that minimized the risk of irritation or injury. Furthermore, participants were closely monitored by trained personnel during the experiment to address any issues that might have arisen.

Collar Design and Material Selection: One of the primary safety measures adopted in this study is the careful design of the wearable collar and the selection of materials used in its construction. The collar is made from hypoallergenic and biocompatible materials to minimize the risk of skin irritation or allergic reactions in the participants. Additionally, the design ensures that the collar fits comfortably around the neck, without causing undue pressure or constriction that could lead to choking or discomfort.

Pre-Experiment Assessments: Prior to commencing the experiments, participants were subjected to a pre-test survey, which assessed their overall health and physical ability, particularly in relation to swallowing. This evaluation process allowed the researchers to identify any potential health concerns or contraindications that might pose a risk during the experiment and exclude such individuals from participating.

Fitting and Adjustment: To ensure the safe and comfortable placement of the wearable collar, trained personnel were responsible for fitting the device onto the participants' necks, positioning the sensors appropriately, and making any necessary adjustments. This not only maximized the accuracy of the data collected but also reduced the risk of discomfort or injury due to incorrect placement or pressure on sensitive areas of the neck.

Supervision and Monitoring: Throughout the experiment, participants were closely monitored by the research team to promptly address any issues or concerns that might have arisen. The experimental setup and procedure were designed to minimize the risk of adverse events, and the researchers were prepared to intervene immediately in case of any safety concerns.

Comfortability Survey: After completing the test, participants were asked to complete a comfortability survey to provide feedback on their experience with the wearable collar. This allowed the researchers to identify any areas of discomfort or potential safety concerns and make adjustments or improvements to the device as needed.

Data Visulization and Extracted Features: The feature extraction process was performed using filtered sensor data. As described in previous sections, an array of time-domain and frequency-domain features is computed using a custom MATLAB function, which captures various aspects of the swallowing patterns. These features, such as standard deviation, dip amplitude, time duration, and spectral centroid, among others, provide valuable information for the machine learning algorithm to classify and analyze swallowing events effectively.

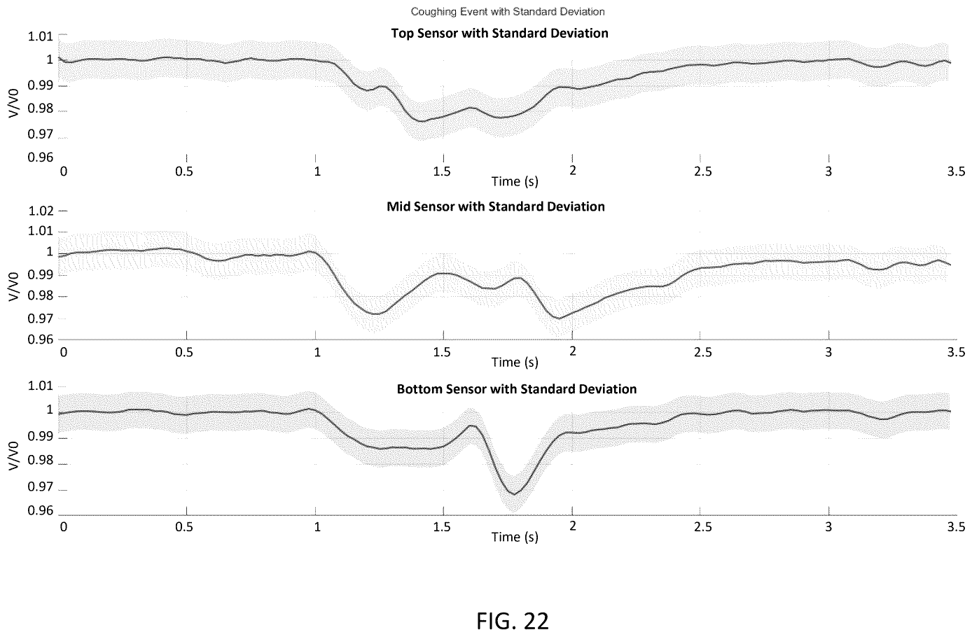

In FIG. 22, the data visualization of extracted features includes the representation of standard deviation for the top, mid, and bottom sensor signals during a coughing event. The shaded regions in the plots represent the standard deviation, providing insight into the variability and dispersion of the sensor data. The calculated standard deviations for top, mid, and bottom sensor signals are 0.0073, 0.0090, and 0.0076, respectively, for the specific set of sensor data only.

The standard deviation data visualization is achieved using the ‘fill’ function in MATLAB. The function is used to create filled polygons with vertices defined by the time vector and the sensor data added and subtracted by the standard deviation values. These polygons effectively represent the shaded regions in the plots, allowing for a visual assessment of the variability in the data.

This visualization technique provides a quantitative measure of the consistency and dispersion of the swallowing events' sensor data. By analyzing the variability of the signals, researchers can better understand the quality of the extracted features and determine the degree of confidence in the classification and analysis of swallowing patterns. Moreover, the standard deviation can assist in the identification of anomalies or outliers that may require further investigation.

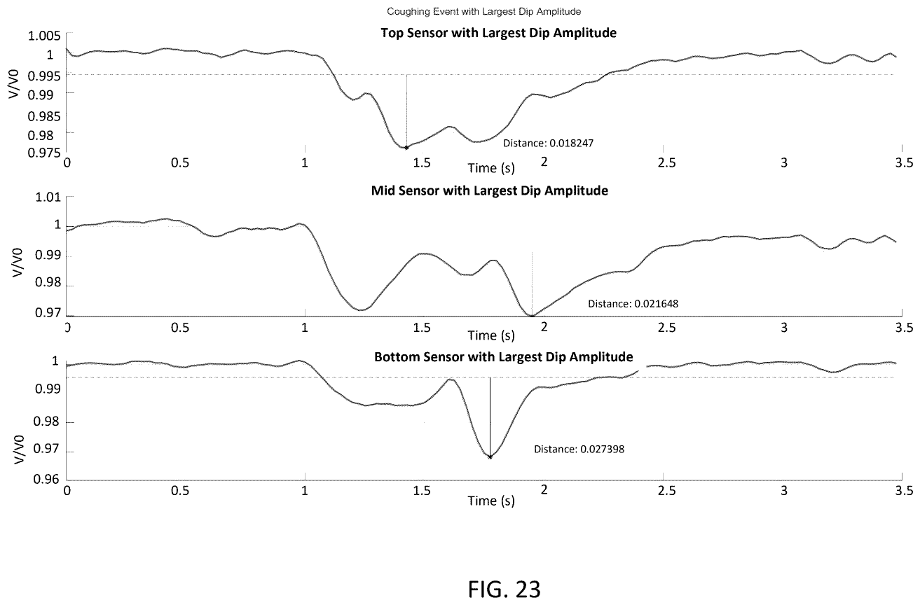

In FIG. 23, the data visualization of extracted features highlights the dip amplitude in the sensor signals for top, mid, and bottom sensors during a coughing event. The calculated dip amplitudes for top, mid, and bottom sensor signals are 0.018247, 0.021648, and 0.027398, respectively. Dip amplitude represents the difference between the mean signal value and the minimum signal value, which can be useful for identifying swallowing events and their characteristics.

The representation of dip amplitudes assists in the identification of swallowing events and contributes to the development of more effective classification algorithms. By analyzing the dip amplitudes, researchers can detect swallowing events with different intensity levels and understand their underlying characteristics. Moreover, the analysis of dip amplitude can help detect anomalies and assess the overall quality of the extracted features.

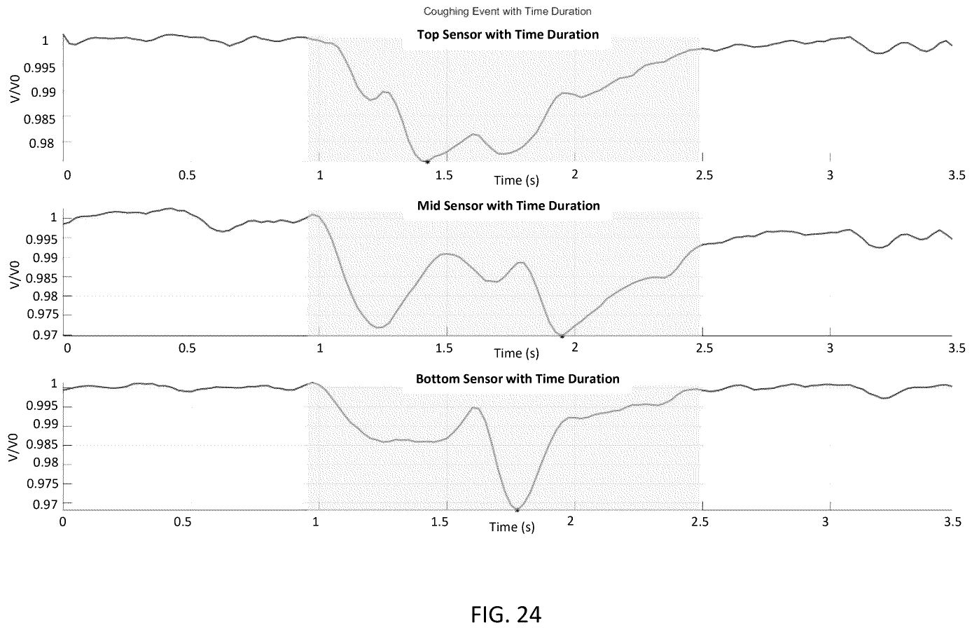

In FIG. 24, the data visualization of extracted features emphasizes the time duration of swallowing events, a critical factor in understanding and analyzing swallowing patterns. The time duration for top, mid, and bottom sensor signals were found to be 1.54 seconds each, as demonstrated by the shaded region in the respective plots.

The time duration is determined by first computing the slope between five data points, which helps in reducing the noise impact. The start threshold is set at a slope greater than 0.04, and the duration continues until the stop threshold is met. The stop threshold is defined as any slope less than 0.01. The region between the start and stop thresholds is shaded to indicate the time duration of interest.

Visualizing time duration in this manner allows researchers to better comprehend the dynamics of swallowing events and contributes to the development of more effective classification algorithms. By analyzing the time duration, researchers can identify the different phases of swallowing events, detect anomalies, assess the overall quality of the extracted features, and refine the device's performance and usability.

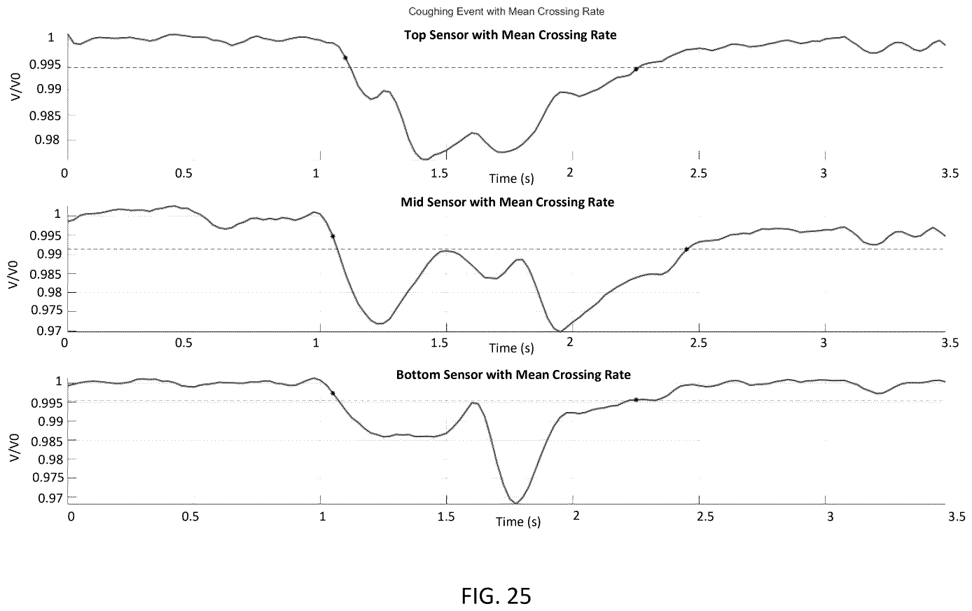

In FIG. 25, this section elaborates on the data visualization of the mean crossing rate, a crucial extracted feature to assess the sensor signal's behavior in relation to swallowing events. The mean crossing rate represents the number of times the signal crosses its mean value within a specified time interval. For top, mid, and bottom sensors, the crossing rates were 2, 2, and 2 times, respectively, as indicated by the data markers in the plots.

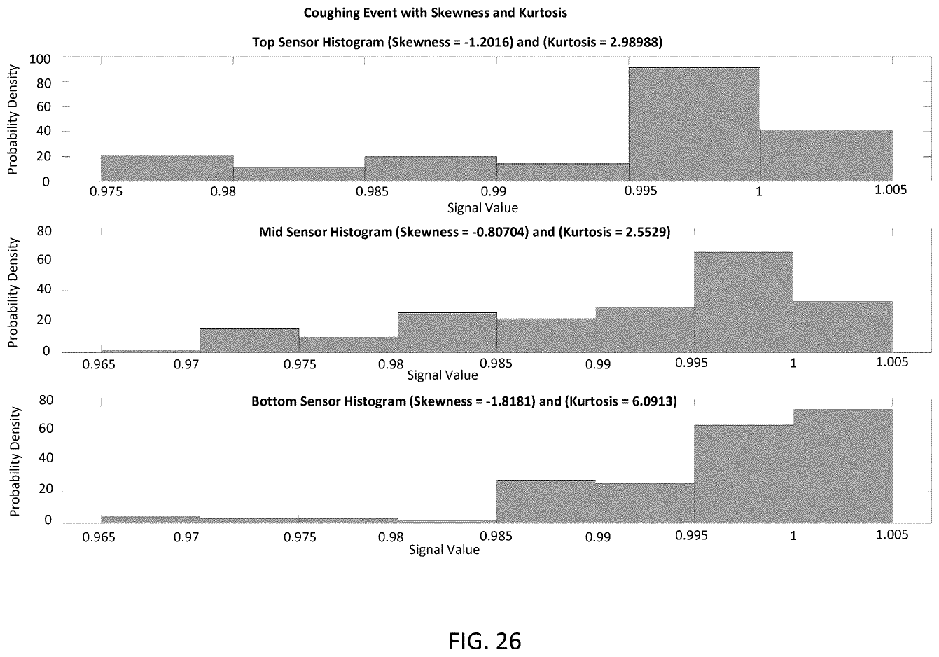

In FIG. 26, this section focuses on the data visualization of skewness and kurtosis, critical extracted features that evaluate the sensor signal's distribution characteristics. Skewness measures the asymmetry of the probability distribution, while kurtosis describes the distribution's “tailedness” or the concentration of data points around the mean.

The visualization process for skewness and kurtosis starts by generating histograms of the top, mid, and bottom sensor signals, normalized by probability density. Skewness and kurtosis values are calculated for each sensor signal using built-in MATLAB functions. For top, mid, and bottom sensors, the skewness values were −1.2016, −0.80704, and −1.8181, while the kurtosis values were 2.9898, 2.5529, and 6.0913, respectively.

These values are incorporated into the histogram plot titles, providing a comprehensive visualization of the data distribution for each sensor. A negative skewness value indicates a left-skewed distribution, whereas a kurtosis value greater than 3 suggests that the distribution has more substantial tails than a normal distribution. Analyzing these values can help identify patterns and irregularities in the sensor signals, thereby improving the machine learning algorithm's ability to classify and analyze swallowing events and enhancing the wearable sensing device's performance and utility for potential dysphagia assessment and management.

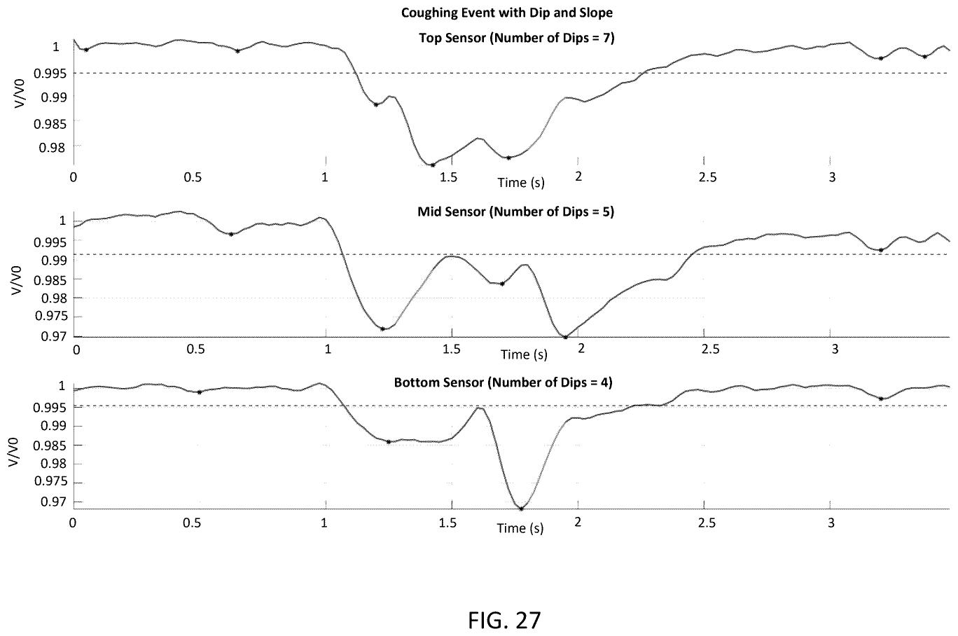

In FIG. 27, this section concentrates on the data visualization of two essential extracted features: the number of dips and the slope. These features help characterize swallowing events and aid in the classification process. Dips represent local minima, while the slope measures the signal amplitude's rate of change over time.

The number of dips is identified using the find peaks function, with a prominence threshold defined as 0.2 times the standard deviation for each respective sensor. The number of dips for the top, mid, and bottom sensors are 7, 5, and 4, respectively. These dips emphasize the local minima within the signal, providing insight into swallowing event dynamics.

The maximum slope for the top, mid, and bottom sensors are 0.0982, 0.1034, and 0.1737, respectively. The slopes are calculated using the diff function in MATLAB and divided by the difference in time. A sliding window of 5 data points is employed to obtain the average slope in each window. The steepest slope sections are visualized by plotting a green line over the window with the highest average slope.

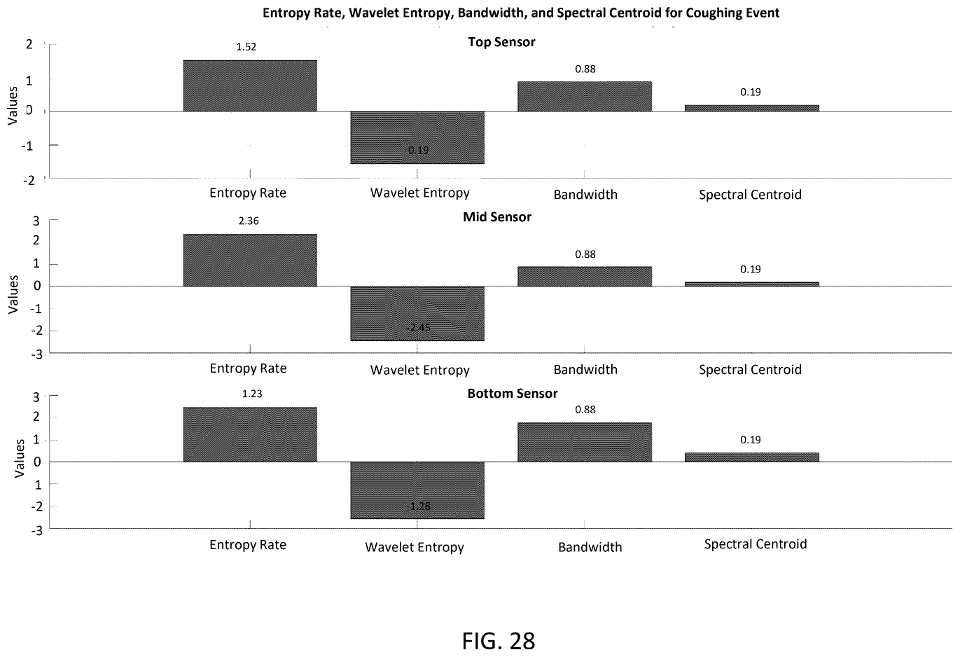

In FIG. 28, the data visualization process for the extracted features focuses on four primary parameters: entropy rate, wavelet entropy, bandwidth, and spectral centroid. These parameters were calculated for each sensor signal (top, mid, and bottom) to evaluate and analyze their distinct characteristics during swallowing and non-swallowing events.

Entropy rate, or Shannon entropy, quantifies the randomness or unpredictability in the given data, serving as a crucial measure of the signal's complexity. The values calculated for top, mid, and bottom sensors were 1.52, 2.36, and 1.23, respectively. Higher entropy rates indicate increased complexity in the signal patterns, which can signify various swallowing conditions or challenges.

Wavelet entropy represents the signal's energy distribution across different frequency bands, capturing localized changes in frequency over time. The wavelet entropy values for top, mid, and bottom sensors were −1.57, −2.45, and −1.28, respectively.

Bandwidth measures the range of frequencies present in the sensor signal, providing insights into the signal's frequency content. The bandwidth values were consistent for all three sensors at 0.88, reflecting similar frequency distribution in the detected signals.

Spectral centroid characterizes the signal's “center of mass” in the frequency domain, giving a measure of the signal's perceived brightness. The values obtained for top, mid, and bottom sensors were 0.19, 0.19, and 0.19, respectively, implying similar brightness perception in all three sensor signals.

Feature Selection and Ranking: Disclosed herein are various features that were extracted from the data collected using the wearable sensing device to capture relevant information about swallowing and non-swallowing actions. The feature selection process aimed to identify the most relevant and informative features that could effectively distinguish between different swallowing patterns. The selected features are described below in terms of their technical, mathematical, and engineering aspects, as well as their relation to the swallowing process

TABLE 6
Feature Rankings for each Sensor

| | | Middle Sensor | | Bottom Sensor | |
|---|---|---|---|---|---|
| Top Sensor Features | | Features | | Features | |
| Time | 70.57 | Bandwidth | 72.69 | Bandwidth | 65.22 |
| Duration | | | | | |
| Spec Centd | 57.05 | Spec Centd | 60.94 | AUC | 63.44 |
| AUC | 56.49 | AUC | 60.37 | Time | 58.52 |
| | | | | Duration | |
| Bandwidth | 53.46 | Time Duration | 58.52 | Spec Centd | 57.18 |
| Min Dip | 35.77 | Min Dip | 38.67 | Skewness | 43.88 |
| Entropy Rate | 30.18 | Skewness | 38.16 | Mean Prom | 35.32 |
| Wavelet | 30.1 | Std | 32.96 | Min Dip | 32.11 |
| Entropy | | | | | |
| Std | 29.57 | Wavelet | 25.57 | Num of | 25.69 |
| | | Entropy | | Dips | |
| Max Slope | 27.47 | Mean Prom | 25.02 | Std | 24.48 |
| Min Slope | 21.76 | Entropy Rate | 24.54 | Entropy | 20.9 |
| | | | | Rate | |
| Mean Prom | 19.82 | Min Slope | 17.09 | Wavelet | 20.82 |
| | | | | Entropy | |
| Num of Dips | 19.64 | Num of Dips | 15.49 | Min Slope | 14.58 |
| Mean Cross | 11.53 | Max Slope | 13.88 | Mean Cross | 13.72 |
| Rate | | | | Rate | |
| Skewness | 8.514 | Mean Cross | 11.41 | Max Slope | 13.72 |
| | | Rate | | | |
| Dip Amp | 7.676 | Dip Amp | 5.872 | Dip Amp | 6.785 |
| Max Dip | 7.676 | Max Dip | 5.872 | Max Dip | 6.785 |
| Kurtosis | 6.886 | Kurtosis | 0.807 | Kurtosis | 4.356 |

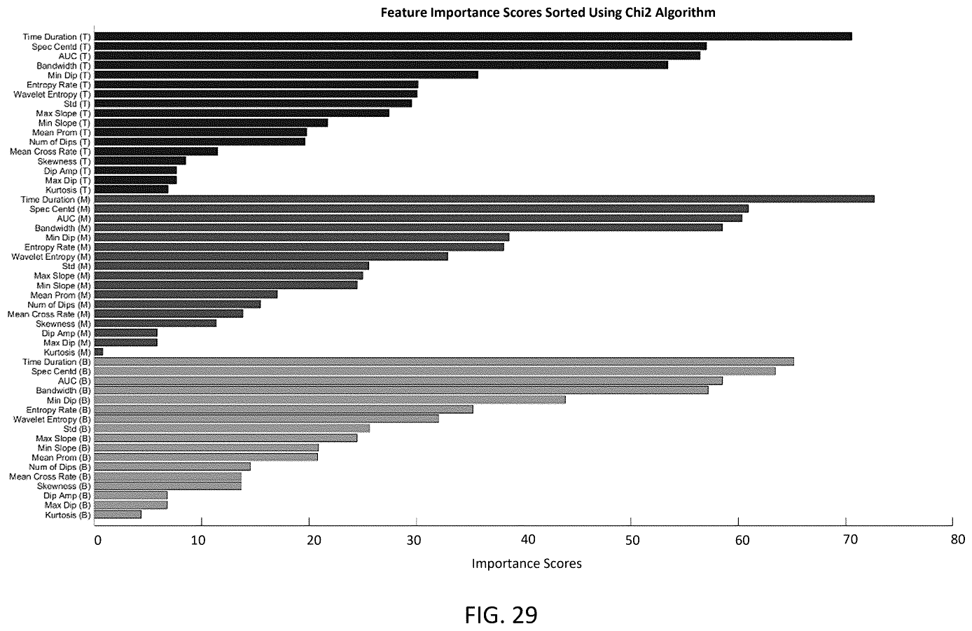

The feature ranking process was conducted using MATLAB's Classification Learner App, employing the Chi2 algorithm to assign an importance score to each feature extracted from the sensor data of healthy subjects performing either non-swallowing or swallowing actions (FIG. 29). The Chi2 algorithm is widely used for feature selection in machine learning tasks due to its simplicity and computational efficiency. It ranks features based on their relevance to the classification task, allowing researchers to identify the most informative features for a given problem. In the disclosed study, the Chi2 algorithm helps us understand the relationship between the features and the swallowing process in healthy participants, enabling the identification of key features that contribute to differentiating swallowing and non-swallowing actions. The top-ranked features for each sensor are presented in this section, along with an analysis of their relevance to the action of swallowing and the underlying mechanics.

The feature ranking process has identified several key features that contribute to the accurate classification of swallowing and non-swallowing events in healthy subjects. These features are related to the temporal, spectral, and energy aspects of the swallowing signal, which are all indicative of the complex biomechanics and muscle coordination involved in swallowing. Understanding the importance of these features not only aids in the development of more effective classification algorithms for differentiating swallowing and non-swallowing actions but also provides valuable insights into the underlying physiological mechanisms of swallowing in healthy individuals.

By analyzing the ranked features, the importance of temporal characteristics such as time duration can be inferred, which is indicative of the efficiency of swallowing in healthy subjects. Additionally, spectral features, such as the spectral centroid and bandwidth, provide information about the coordination and complexity of muscle activation during swallowing. Energy-based features, such as the area under the curve (AUC), can be used to assess the intensity of muscle activation, which is a critical aspect of swallowing function.

These findings suggest that an optimal combination of temporal, spectral, and energy features can provide a comprehensive and robust representation of the swallowing process in healthy subjects. Future work in this field should focus on exploring additional features and feature selection methods to further enhance the accuracy and efficiency of swallowing event detection and assessment, eventually enabling the development of tools for dysphagia screening and monitoring.

Machine Learning Model Comparison and Evaluation: To evaluate the performance of different machine learning models, various metrics can be calculated from a confusion matrix. A confusion matrix is a table that shows the true positive (TP), false positive (FP), true negative (TN), and false negative (FN) values for a binary classification problem. The confusion matrix can be extended to multi-class classification problems as well.

Here's an explanation of how to calculate various performance metrics from a confusion matrix:
1. Accuracy: It is the proportion of correctly classified instances out of the total instances. Formula: Accuracy=(TP+TN)/(TP+TN+FP+FN)
2. Sensitivity (Recall): It is the proportion of actual positive instances that were predicted as positive. Formula: Sensitivity=TP/(TP+FN)
3. Specificity: It is the proportion of actual negative instances that were predicted as negative. Formula: Specificity=TN/(TN+FP)
4. Precision: It is the proportion of predicted positive instances that were actually positive. Formula: Precision=TP/(TP+FP)
5. F1-score: It is the harmonic mean of precision and recall, which provides a balance between them. Formula: F1-score=2*(Precision*Sensitivity)/(Precision+Sensitivity)

The machine learning models employed for classifying swallowing events include Fine K-Nearest Neighbors (Fine KNN), Cubic Support Vector Machine (Cubic SVM), and Medium Neural Network. The performance of these models is evaluated based on the accuracy, sensitivity, specificity, and other relevant performance metrics calculated from each model's confusion matrix.

Fine KNN: The Fine KNN algorithm classifies instances based on the majority class of its K-nearest neighbors in the feature space. It is a non-parametric, lazy learning method that makes no assumptions about the underlying data distribution. Strengths: Fine KNN is simple, easy to implement, and has relatively low computational requirements during training. It is also robust to noisy data and can capture complex decision boundaries. Limitations: Fine KNN's performance is sensitive to the choice of K and the distance metric. It also suffers from the curse of dimensionality and has high computational complexity during inference due to the need to compute distances to all training instances.

Cubic SVM: The Cubic SVM algorithm constructs a decision boundary (a hyperplane) that separates the classes in the feature space while maximizing the margin between them. The Cubic SVM employs a cubic kernel function to transform the data into a higher-dimensional space, making it possible to find a separating hyperplane. Strengths: SVMs can efficiently handle high-dimensional data and are less prone to overfitting. The kernel trick allows for modeling complex, non-linear relationships between features. Limitations: SVMs are sensitive to the choice of kernel function and hyperparameters. They can have high computational requirements for large datasets and can be challenging to interpret and explain.

Medium Neural Network: The Medium Neural Network is an artificial neural network with one or more hidden layers, capable of modeling complex relationships between input features and output classes. Strengths: Neural networks are universal function approximators, capable of modeling highly complex, non-linear relationships between features. They can be fine-tuned by adjusting the architecture and training hyperparameters. Limitations: Neural networks require large amounts of data and extensive computational resources for training. They can be prone to overfitting and are often considered “black-box” models, making their inner workings challenging to interpret.

TABLE 7
Machine Learning Model Results

| | | | Medium |
|---|---|---|---|
| | Fine | Cubic | Neural |
| Model | KNN | SVM | Network |
| Accuracy | 75.60% | 67.50% | 56.30% |
| Precision | 72.40% | 59.10% | 48.20% |
| Sensitivity | 81.50% | 68.40% | 59.90% |
| Specificity | 74.40% | 68.00% | 55.10% |
| F1-score | 76.70% | 63.40% | 53.40% |

Comparing the results of the three models (Table 7), Fine KNN demonstrates the best performance in terms of accuracy, sensitivity, and F1-score. The high sensitivity of Fine KNN suggests that it is particularly effective in detecting true swallowing events, which is crucial for dysphagia assessment. Cubic SVM performs moderately well, while the Medium Neural Network has the lowest accuracy and F1-score.

In light of the performance metrics and the strengths and limitations of each model, Fine KNN emerges as the best choice for this application. However, it is essential to optimize the choice of K and the distance metric to ensure the best performance.

Developing a high-performing machine learning model necessitates the careful tuning of hyperparameters. This process involves determining the optimal combination of hyperparameters, which can significantly influence the model's performance (Table 8). In this study, the k-Nearest Neighbors (KNN) algorithm was employed for classification, and its hyperparameters were carefully tuned to balance the classification accuracy and computational complexity.

TABLE 8
Hyperparameters for
the disclosed Model
Model Hyperparameters

| | Fine |
|---|---|
| Preset | KNN |
| Number of | 1 |
| Neighbors | |
| Distance Metric | Euclidean |
| Distance Weight | Equal |
| Standardize Data | Yes |

The KNN algorithm's hyperparameters include the following:
Number of Neighbors (k): This hyperparameter determines the number of neighboring data points considered when making a classification decision. A smaller value of k results in a more flexible model, which can capture intricate patterns in the data but may be prone to overfitting. Conversely, a larger value of k yields a smoother decision boundary, potentially increasing the model's generalization ability but potentially sacrificing sensitivity to local patterns. In this study, a value of k=1 was chosen after evaluating different values of k using cross-validation. The choice of k=1 provided the best performance in accuracy, precision, and F-1 score ensuring accurate classification while minimizing the risk of overfitting.
Distance Metric: The distance metric is used to compute the distance between data points in the feature space. Common distance metrics include Euclidean, Manhattan, and Mahalanobis distances. The choice of distance metric can influence the model's performance and should be selected based on the data characteristics and the problem's nature. In this study, the Euclidean distance metric was employed because it is invariant to rotation, making it suitable for detecting differences in feature space across various orientations. Furthermore, the Euclidean distance is computationally efficient and provides satisfactory performance in practice.
Distance Weight: This hyperparameter determines the weighting scheme applied to the neighbors' votes. When using equal weighting, each neighbor contributes equally to the classification decision. Alternatively, distance weighting schemes, such as inverse distance or Gaussian weighting, assign higher importance to closer neighbors. In this study, equal weighting was chosen after comparing the performance of various weighting schemes. Equal weighting yielded similar classification accuracy to more complex weighting schemes while requiring fewer computational resources, making it a suitable choice for this application.
Standardize Data: Standardizing the data involves scaling the feature values to have zero mean and unit variance. This process ensures that all features contribute equally to the distance calculation, preventing features with larger magnitudes from dominating the classification decision. In this study, the data were standardized to account for potential differences in feature magnitudes and measurement units, leading to improved model performance.

The KNN classifier was rigorously tuned by training and evaluating it on a variety of hyperparameter combinations using 10-fold cross-validation, which means that 90% of the data was used for training, and 10% was set aside for testing in each iteration. This systematic method, known as grid search or parameter sweep, is instrumental for optimal hyperparameter selection.

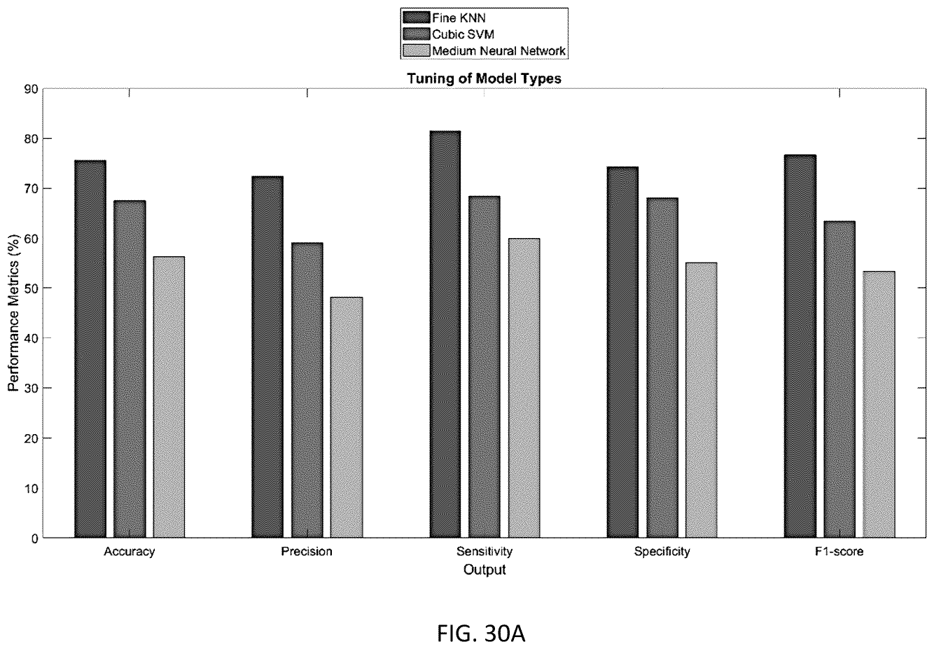

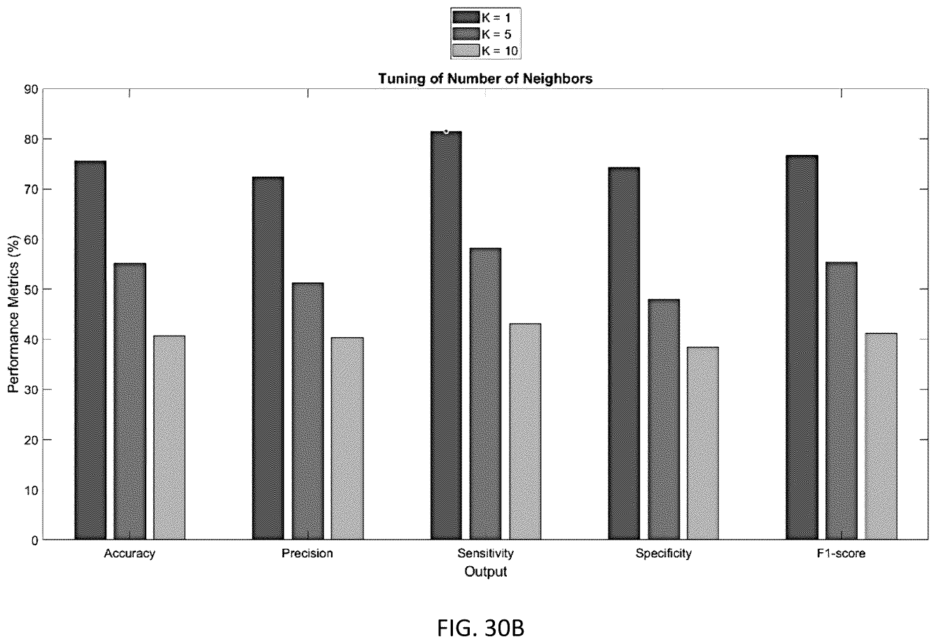

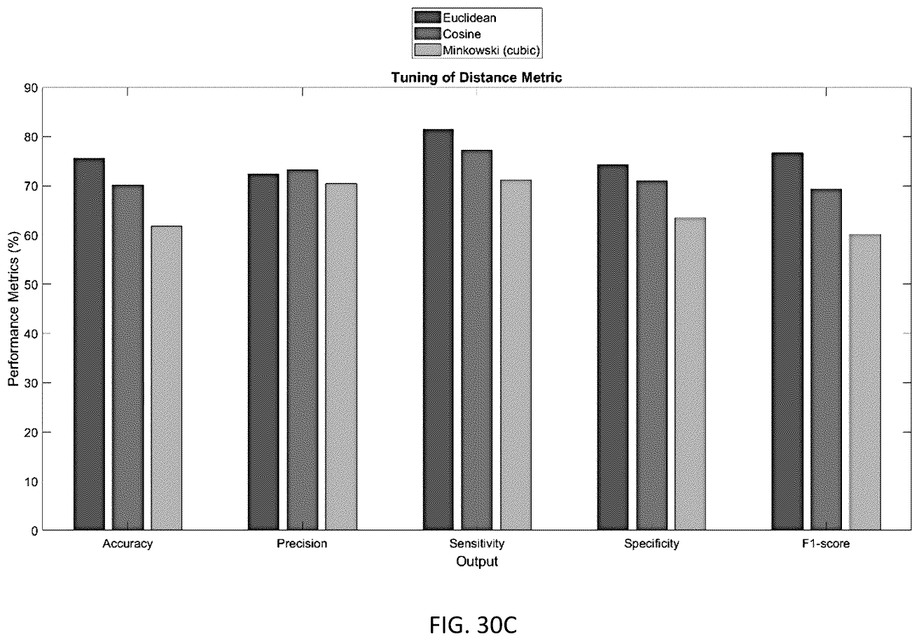

A prominent feature of the disclosed tuning process was the variation in the number of neighbors (K) in the KNN algorithm. The classifier performance metrics, such as Accuracy, Precision, Sensitivity, Specificity, and F1-score, were observed to decrease as the value of K increased from 1 to 10, indicating that a lower value of K yielded better results for the specific application (see FIG. 30B).

The selection of distance metric also played a substantial role in the disclosed model performance. The Euclidean distance metric led to the best performance, with Accuracy of 75.6%, Precision of 72.4%, Sensitivity of 81.5%, Specificity of 74.4%, and F1-score of 76.7%. Cosine and Minkowski (cubic) metrics showed slightly lower performance, indicating that the choice of distance metric has a weaker influence on the model's outcomes compared to the number of neighbors and model type (as represented in FIG. 30C).

The disclosed classifier's model type was another critical hyperparameter, with Fine KNN achieving the highest scores across all performance metrics. Cubic SVM and Medium Neural Network configurations, though still adequate, performed less well (FIG. 30A).

Overall, the carefully selected hyperparameter combinations led to a model with high classification accuracy and acceptable computational complexity. This result illustrates the potential of the disclosed wearable sensing device and algorithm for differentiating swallowing and non-swallowing actions in healthy subjects.

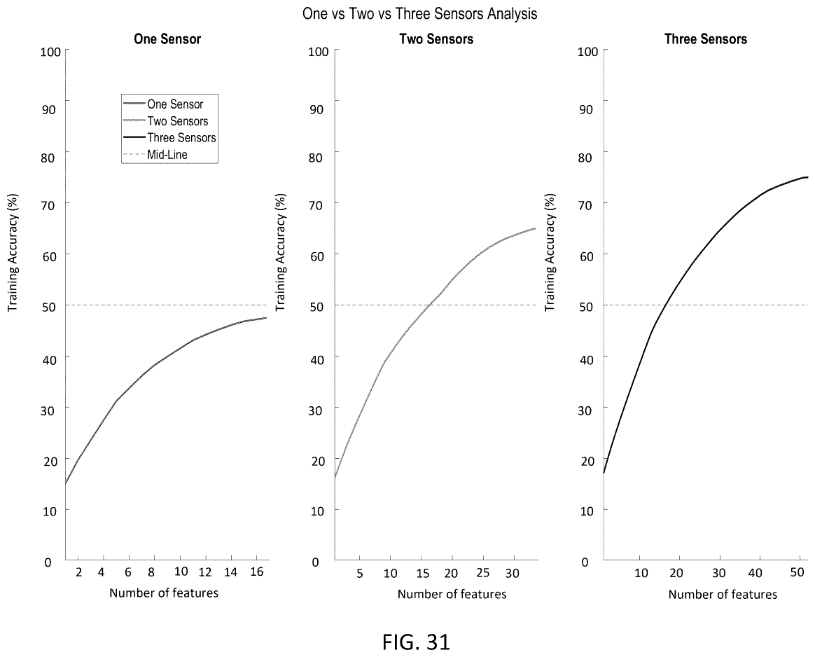

One vs Two versus Three Sensors Analysis: Disclosed herein is the analysis of the impact of using one, two, and three sensors for detecting swallowing and non-swallowing events and classifying specific actions. The wearable sensing device's performance was assessed by calculating the training accuracy based on the number of features used for each configuration (FIG. 31).

In order to enhance the granularity of the disclosed analysis, an in-depth examination of various combinations of sensor configurations was utilized, specifically single, dual, and triple sensor setups, as presented in Table 6. In the case of a single sensor arrangement, the top sensor was deployed, which demonstrated superior performance characteristics in the included trials. For the dual sensor configuration, both the top and middle sensors were utilized, while in the three-sensor setup, the top, middle, and bottom sensors were concurrently implemented. These configurations were chosen from a wide array of possibilities, based on their superior performance metrics and effectiveness in capturing the required biomechanical features.

The methodological framework for escalating the number of features was rooted in an iterative process that integrated sensor features in a prioritized manner, dictated by their importance scores. The relevance of each feature was quantified using a Chi2 test, which evaluated the contribution of each feature to the overall accuracy of the classification algorithm. Features with higher importance scores, indicating a stronger contribution to the classification process, were incorporated first, and subsequent features were added in descending order of their scores until the least contributory features were integrated.

The result of this process was the generation of an accuracy curve for each sensor setup, displaying the accuracy as a function of the number of features included in the analysis. As illustrated in FIG. 31, this accuracy curve offers valuable insights into the influence of an incremental number of features on the overall performance of the sensor setup, providing a clear visualization of the point of diminishing returns, where the addition of more features does not significantly enhance the accuracy of the system. Using a single sensor positioned on the larynx, or Adam's apple, resulted in a cross-validation accuracy of 47.6% with 17 features. This level of accuracy is relatively low, as it might not be sufficient to reliably detect swallowing events or differentiate them from other activities. One possible explanation for the limited performance is the high degree of variability in the biomechanics of swallowing, which might be challenging to capture using only one sensor. Moreover, a single sensor might be more susceptible to interference from head and body movements or environmental noise, making it less reliable for clinical applications.

When incorporating a second sensor placed above the larynx, near the hyoid bone, the training accuracy significantly increased to 65.2% using 34 features. This improvement indicates that the additional sensor provides valuable information about the complex dynamics and muscle coordination involved in swallowing. The elevation of the larynx plays a crucial role in the swallowing process, as it moves upward and forward during swallowing, contributing to the opening of the upper esophageal sphincter. Thus, monitoring its movement with a sensor can improve the system's performance in detecting and classifying swallowing events.

The highest training accuracy of 75.6% was achieved when using three sensors, with the third sensor placed below the larynx to monitor head or body movements. By employing 51 features, this configuration allows the system to account for potential confounding factors such as head or body movement, which could otherwise lead to false positives or negatives. The increased accuracy indicates that the third sensor enhances the specificity and sensitivity of the system, resulting in a more robust and reliable detection of swallowing events.

However, it is essential to consider the trade-offs associated with using multiple sensors. While the additional sensors improve accuracy, they may also increase the complexity of the system, making it more challenging to deploy, set up, and maintain. Furthermore, the increased number of sensors may lead to higher computational requirements, which could impact the real-time performance of the system. Therefore, it is crucial to balance the benefits of improved accuracy with the practical considerations of using multiple sensors.

The analysis of one, two, and three sensors demonstrates that the use of multiple sensors can significantly enhance the wearable sensing device's performance in detecting swallowing and non-swallowing events and classifying specific actions. The improved accuracy, sensitivity, and specificity achieved with three sensors make this configuration the most suitable for clinical applications in dysphagia assessment and management. Future research should focus on optimizing the placement and number of sensors to minimize complexity and computational requirements while maintaining high accuracy.

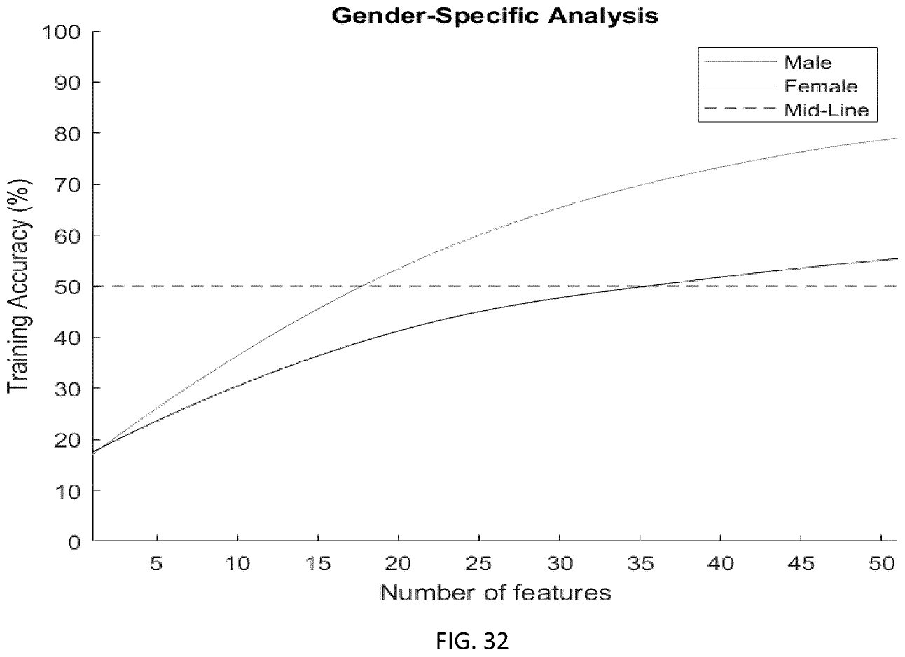

Disclosed herein is the analysis of the performance of the wearable sensing device when separating male and female participants. The three-sensor configuration was employed, and the training accuracy was reported for each gender. Understanding gender-specific trends or differences in the data can provide valuable insights for improving swallow assessment and classification, as well as the design of the wearable sensing device (FIG. 32—Gender-Specific Analysis).

The analysis of male participants resulted in a training accuracy of 78.8% with 51 features. This relatively high accuracy indicates that the three-sensor configuration effectively captures the complex biomechanics and muscle coordination involved in swallowing for male subjects. On the other hand, the training accuracy for female participants was significantly lower at 55.4%. This discrepancy can be attributed to a few factors, which will be discussed below.

One potential factor contributing to the reduced accuracy for female participants is the anatomical differences between males and females. Female neck circumference and height are generally smaller than males, which can impact the fit and sizing of the wearable garment. The prototype used in this study did not have various sizes available, and the adjustable button mechanism may not have provided sufficient flexibility to accommodate these differences. In future iterations of the wearable sensing device, it would be essential to consider a more adaptable design that accommodates various neck sizes and anatomical variations to ensure proper sensor placement and reliable signal acquisition.

Another critical difference between male and female participants is the prominence of the larynx or Adam's apple. In general, the larynx is less pronounced in females, which could impact the quality of the signals captured by the first sensor placed on the larynx. This variation could lead to a higher degree of noise or interference in the data, resulting in lower accuracy for female participants. Further research is needed to optimize sensor placement and configuration for both male and female subjects to enhance the system's performance across genders.

The observed gender-specific trends and differences in the data have important implications for dysphagia assessment and management. First, these findings highlight the need for personalized approaches that consider individual anatomical variations when developing wearable sensing devices for swallowing assessment. By tailoring the device to each subject, the accuracy and reliability of the system can be improved.

Second, the differences in training accuracy between male and female participants underscore the importance of considering gender-specific factors in the development of machine learning algorithms for swallowing event classification. By incorporating gender-specific features or developing separate models for each gender, it may be possible to improve the accuracy of swallowing event detection and classification for both male and female subjects.

This research has made significant contributions to the understanding and development of wearable sensors for monitoring swallowing. By designing and implementing a wearable sensing device equipped with three strategically placed sensors, the study has demonstrated the feasibility of non-invasive, continuous monitoring of swallowing patterns in real-world settings. This innovative approach offers advantages over traditional clinical assessments, such as videofluoroscopy, which are often limited by invasiveness, radiation exposure, and constrained environments.

The development of machine learning algorithms for the classification and analysis of swallowing events represents another important contribution of this research. By identifying key features associated with swallowing biomechanics, the study has demonstrated the potential for data-driven models to accurately and objectively classify swallowing events. This is particularly valuable for healthcare professionals as it provides a quantitative basis for dysphagia assessment, leading to more informed diagnoses and treatment decisions.

The disclosed findings have the potential to improve dysphagia diagnosis, treatment, and monitoring in several ways. The wearable sensing device enables continuous and real-time monitoring of swallowing patterns, which could allow for earlier detection of dysphagia and timely intervention. The machine learning algorithms developed in this research could be used to identify subtle changes in swallowing patterns that might otherwise go unnoticed, helping to prevent complications and optimize treatment outcomes. By enabling more accurate and efficient swallowing assessment, the device may help patients receive the appropriate treatment sooner and minimize the risk of complications, such as aspiration pneumonia. Furthermore, by allowing patients to monitor their swallowing patterns at home, the device empowers them to take a more active role in managing their condition and facilitates communication with healthcare providers.

For healthcare professionals, the wearable sensing device offers several benefits. By providing objective and quantitative data on swallowing patterns, the device could complement existing assessment methods and enhance the accuracy of dysphagia diagnoses. The machine learning algorithms developed in this research can also help professionals better understand the complex biomechanics of swallowing and identify patterns that may be indicative of underlying pathology. Additionally, the continuous monitoring capabilities of the wearable device can improve the long-term management of patients with dysphagia, enabling healthcare professionals to monitor treatment progress and adjust interventions as needed.

In conclusion, this research has made significant contributions to the development of wearable sensors for dysphagia assessment and has the potential to improve the lives of patients with swallowing disorders. By leveraging innovative sensing technology and advanced machine learning algorithms, this work represents a critical step towards more accurate and efficient dysphagia assessment and care.

### Example 5: A Wearable Fabric Sensor to Measure Laryngeal Elevation: a Proof of Concept Study

Purpose: Currently, the only way to quantify laryngeal elevation during swallowing is videofluoroscopy. A wearable device that quantifies amplitude of laryngeal elevation can add to clinical evaluations and biofeedback treatment approaches. We explored the potential for a wearable fabric sensor to detect swallowing and measure laryngeal elevation.

Methods: A knitted collar containing 3 strain sensors (superior: 1 cm above thyroid notch; middle: at thyroid notch; inferior: 1 cm below thyroid notch) using silver-coated yarn was used to collect signals from the neck of 12 healthy adults (6 male, age 18-33) during 5 ml, 20 ml, and effortful liquid swallows, vocalization, cough, head rotation, and quiet resting. Fine k-nearest neighbor (KNN) analysis was used to classify swallowing vs. non-swallowing tasks. RM-ANOVA was used to compare sensor performance on swallowing tasks known to differ with laryngeal elevation. We hypothesized that the KNN could sufficiently differentiate swallowing from non-swallow tasks and that sensor signals would be significantly different in swallows requiring more laryngeal elevation (5 ml vs. 20 ml vs. effortful liquid swallows).

Results: The best-performing KNN model used data from all 3 sensors and had an accuracy of 75.6%, sensitivity of 81.5%, and specificity of 74.4%. The superior sensor had an M-wave negative deflection, reflecting laryngeal movement to and above the sensor during swallowing. Middle and Inferior sensors had unimodal positive deflections, reflecting decreased strain from laryngeal elevation away from the sensor. Signal duration was significantly longer in effortful swallows vs. 5 or 20 ml boluses in the superior sensor only (p=0.002), and signal amplitude was significantly greater in effortful swallows across all sensors (p<0.02).

Conclusions: Findings confirm proof of concept that timing and amplitude of laryngeal excursion can be measured using fabric-based strain sensors placed on the outside of the neck. An increased number of sensors may provide more specific data on excursion amplitude, and validation work will need to be completed using videofluoroscopy.

Additional classification findings and proof of concept that the disclosed knitted fabric sensor device changes signals with different tasks known to affect laryngeal elevation.

### Example 6: The Development of a Wearable Fabric Sensing Device for Swallow Monitoring and Classification

In this work, a novel wearable fabric sensing device designed explicitly for swallow monitoring and classification is introduced. Recognizing the need for a more accessible and real-time monitoring method, this device bridges the gap between traditional clinical evaluations and modern technological advancements. Equipped with three knitted strain sensors, the disclosed device is not only sensitive to the intricacies of the swallowing process but also offers extended monitoring capabilities, capable of transmitting up to 12 hours of continuous data. Leveraging the power of wireless technology, this real-time data is transmitted seamlessly via Bluetooth to a graphical user interface (GUI), ensuring clinicians and caregivers can monitor the swallowing patterns without delay.

One of the significant advantages of the disclosed device lies in its low-profile construction, making it seamlessly integrate with the user's attire without causing discomfort or drawing attention. The use of weft-knitting with a blend of nylon, nylon-wrapped spandex, and silver-coated yarn provides not only flexibility but also durability, ensuring adaptability across various body types and movements [Li, Y., et al., Sensing performance of knitted strain sensor on two-dimensional and three-dimensional surfaces. Materials & Design, 2021. 197: p. 109273]. The recorded real-time data can be archived, providing a valuable repository of signals that can be further classified into distinct features. These features, in turn, serve as the foundational blocks for the disclosed machine learning (ML) model. Remarkably, the disclosed model boasts an accuracy rate exceeding 75% in distinguishing swallowing from non-swallowing patterns. Such precision is commendable and pivotal in ensuring early and accurate detection of dysphagia.

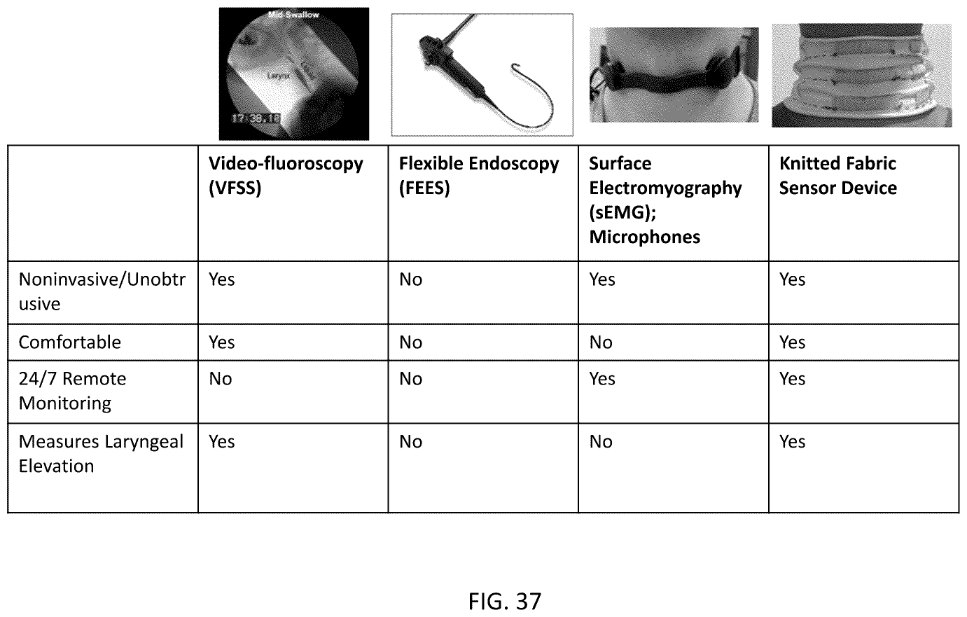

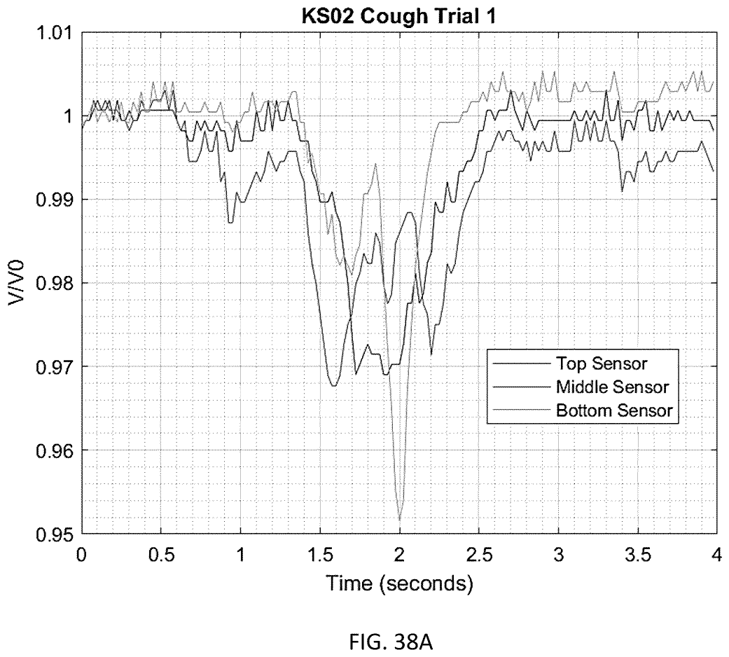

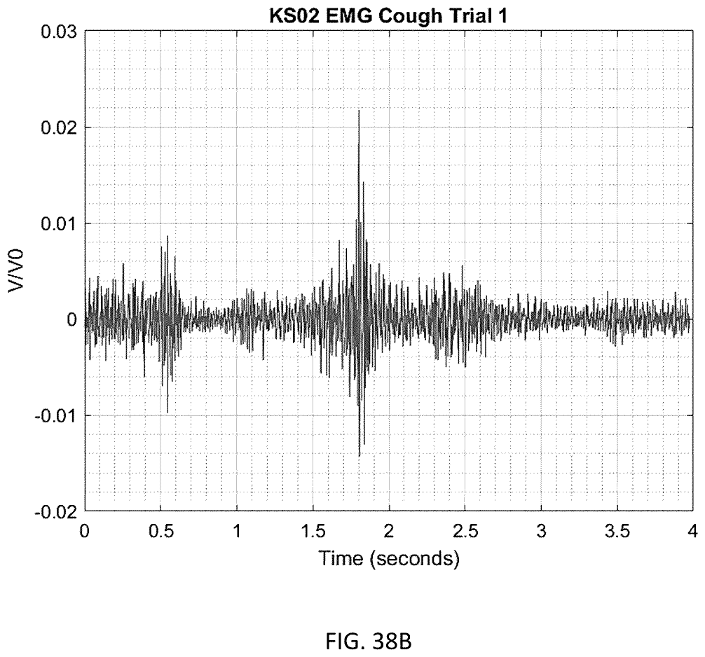

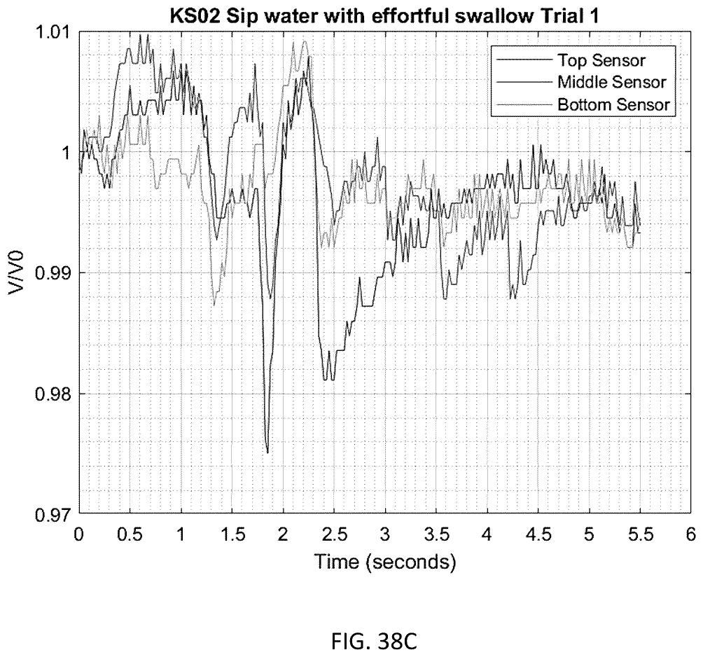

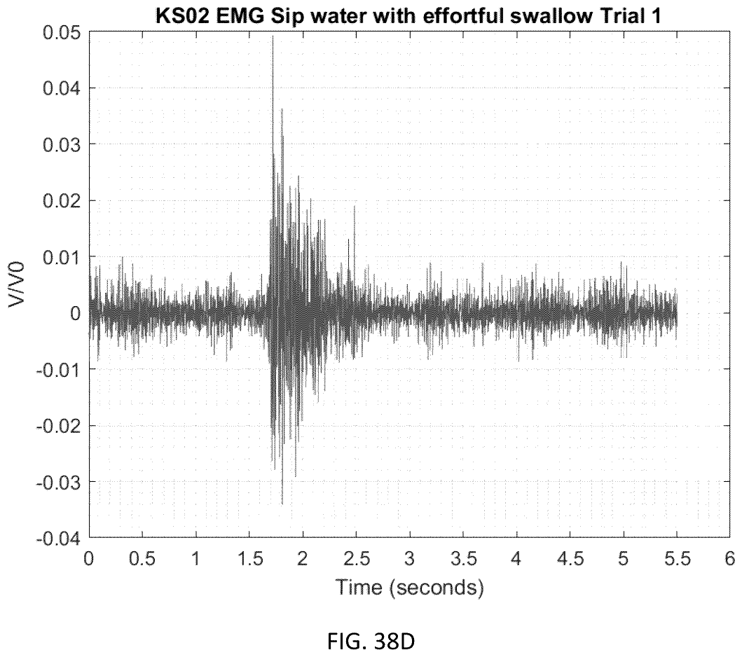

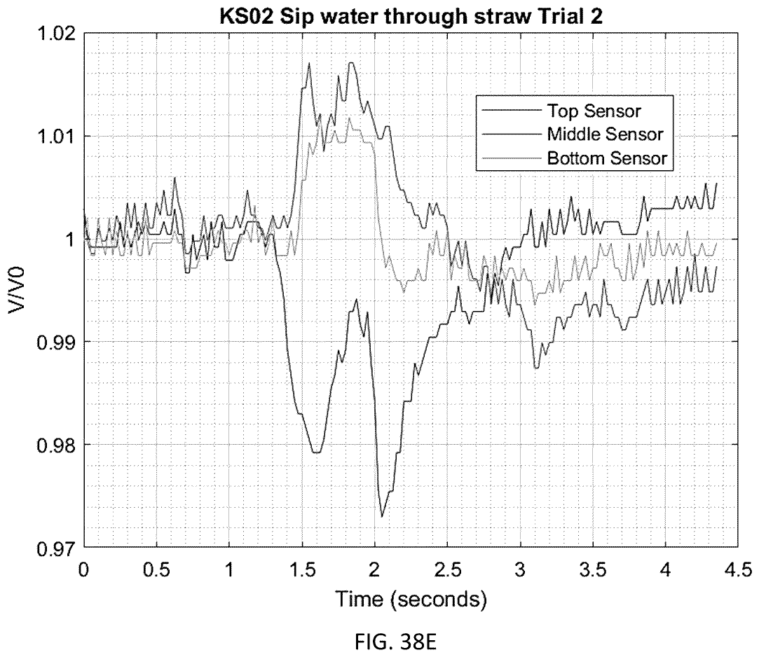

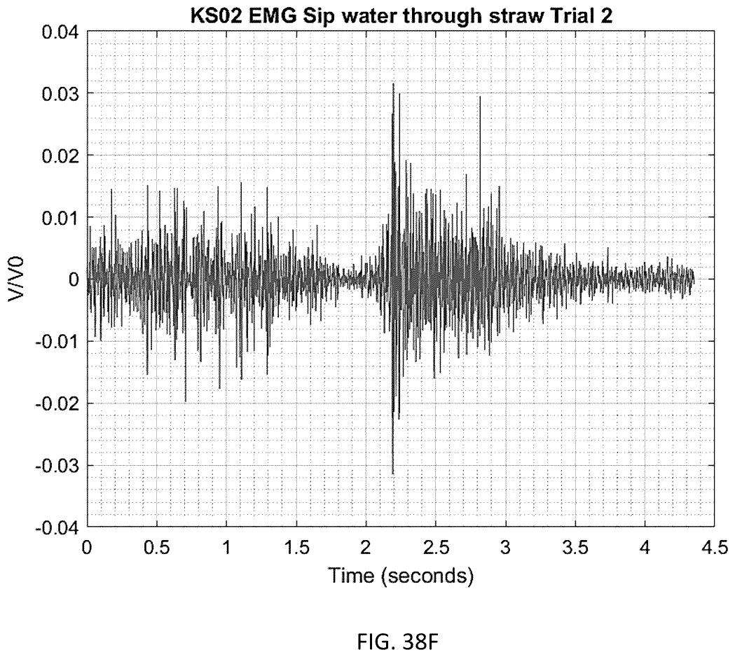

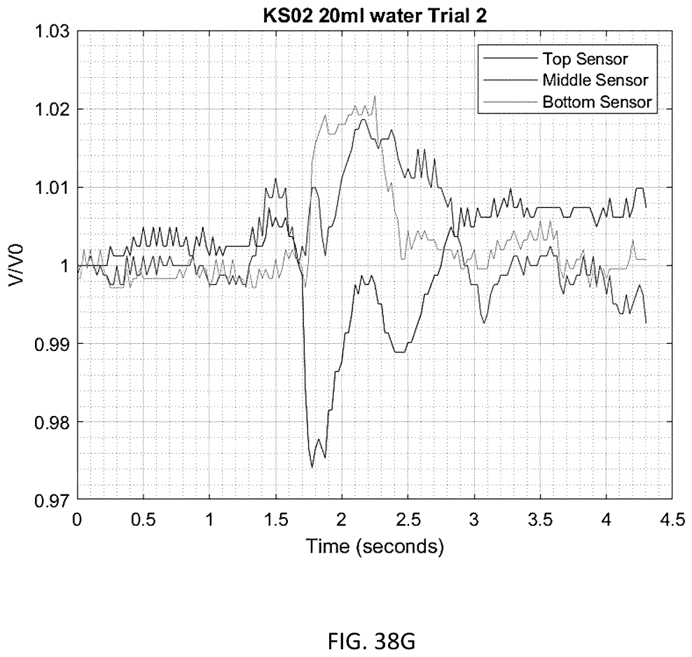

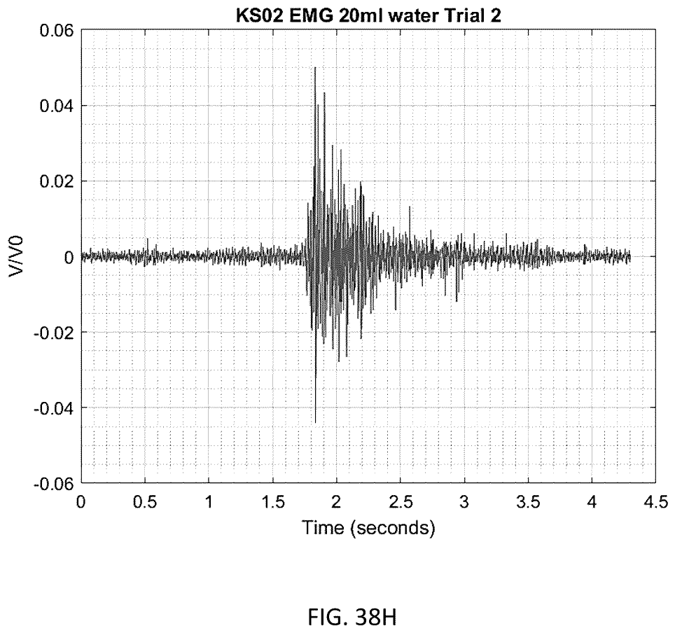

As a pioneering contribution to swallowing diagnostics, this device serves as a proof-of-concept, particularly advantageous for clinical and non-clinical settings. With its promise of continuous, real-time monitoring and ease of use, the disclosed wearable fabric sensing device offers a revolutionary approach, transforming the landscape of dysphagia detection and management. By overcoming the limitations of existing methods (see FIG. 37 comparison), the disclosed invention paves the way for more personalized, efficient, and comprehensive care for individuals susceptible to swallowing disorders.

The wearable fabric collar, designed to provide a comfortable and non-intrusive solution for real-time swallow monitoring, consists of a knitted structure composed of a stable and stretchy fabric base and three strain sensing areas. The base structure is formed by a textured filament nylon yarn (75 dtex, 36f) and a nylon-covered spandex yarn (50 dtex), ensuring comfort and adaptability to various neck sizes and shapes. Mirroring the precision of the traditional three-finger test used in clinical swallow examinations, the strategic placement of the three strain sensors emulates the fingers' positioning, capturing critical dynamics of the swallowing process with engineered precision. The top sensor monitors the upward and forward movement of the larynx, essential for airway protection, while the middle sensor, aligned with the larynx, gauges muscle tension, and symmetry, ensuring the detection of any abnormality or asymmetry in muscle coordination. The bottom sensor, placed below the larynx, assesses the timing and completeness of the swallow. This sensor array effectively replicates the clinician's tactile assessment, providing real-time, quantitative data that reflects the intricacies of the swallowing mechanism. The disclosed novel design allows for the efficient detection of swallowing events, with a reported strain sensing sensitivity reaching 60% for uniaxial tension in the 2D fabric surface, and 120% for all-direction tension in the 3D fabric surface. The response time for the fabric strain sensors is 400 ms for the 2D fabric surface and 350 ms for the 3D fabric surface [Li, Y., et al., Sensing performance of knitted strain sensor on two-dimensional and three-dimensional surfaces. Materials & Design, 2021. 197: p. 109273]. Moreover, the wearable fabric collar is easily connected to the hardware for signal acquisition and data logging. Metal snap buttons are attached to the leads at the ends of the strain sensors, serving as connection points for the mini microcontroller unit (MCU). This ease of connection ensures that the wearable collar can be comfortably and securely worn by users during monitoring.

The integration process begins with the attachment of the hardware components to the wearable fabric collar. The metal snap buttons on the ends of the sensor leads facilitate the electrical resistance measurement as clamping the button together conjoins the hardware to the fabric sensor system. The knitted sensors are integrated into a voltage divider circuit, resulting in different voltage values corresponding to the changes in strain experienced during swallowing. The MCU, Bluetooth module, and power supply are soldered on a PCB board and enclosed in a compact, discreet housing punch located behind the garment. This housing is attached to the collar, ensuring that the system is unobtrusive and comfortable for the patient during the swallowing assessment. In the context of software integration, the MCU is programmed using the Arduino IDE to acquire, process, and transmit the data from the strain sensors to a MATLAB-based user interface. The Arduino IDE code is designed to read the analog inputs from the three strain sensors and convert them into digital values. These values are then transmitted wirelessly via the Bluetooth module to a customized GUI on a computer for real-time visualization and control. The GUI plays a crucial role in displaying the data received from the Arduino Nano through the Bluetooth module. The GUI enables users to monitor the real-time data from the sensors, control the testing process, and save the measured datasets for further analysis. (See FIG. 1B and FIG. 5A)

The device hardware included a mini microcontroller unit (MCU), a Bluetooth module for wireless data transfer, and a 3.7V battery pack for power supply. The MCU is a compact Elegoo Nano Board based on the ATmega328P, compatible with Arduino Nano V3.0. This board is a powerful yet small component, capable of performing complex data processing tasks and providing the foundation for the system's operations. The wireless transmission is handled by a HiLetgo HC-06 RS232 Wireless Bluetooth Serial RF Transceiver. This module features a 4-pin bi-directional serial channel in slave mode and is compatible with Arduino-based systems. It works with any USB Bluetooth adapter and offers low power consumption, high sensitivity, and small form-factor. The Bluetooth version is V2.0+EDR, and its operating voltage is 3.3V. This module enables efficient wireless data transfer between the wearable device and the user interface, allowing for real-time monitoring and analysis of the collected swallowing data. The power supply for the device is provided by a 2000 mAh TOPUSSE Lithium-Ion Polymer battery, ensuring 12+ hours of continuous and stable operation during testing. The Arduino Nano can be powered via a USB connection, a 6-12V unregulated external power supply (VIN pin), or a 5V regulated external power supply (5V pin). The HC-06 Bluetooth module requires a supply voltage of 3.3V to 6V, and its RX pin logic level is 3.3V, which is not 5V tolerant. These power requirements must be considered when designing the power supply system for the device to ensure optimal performance and component longevity.

Using MATLAB's App Designer, a GUI is developed for real-time data visualization and saving of swallowing datasets. The Arduino Nano, integrated with a 10-bit analog-to-digital converter, translates voltage inputs from strain sensors into integer datasets. The variable resistance of the strain sensors, paired with a fixed 47002 resistor, allows voltage readings as signal outputs. These signals are processed by the Arduino IDE for real-time monitoring. The HC-06 Bluetooth module ensures wireless data transmission at a rate of 40 Hz, supporting continuous output for approximately 12 hours. This module uses a transparent serial communication protocol, compatible with both serial terminals and customized GUIs. With the Bluetooth module integrated, there's no need for additional user coding, ensuring seamless Arduino system synchronization. Data visualization requires a high-performance computing device for lag-free real-time display and communication stability. The Arduino code reads inputs from pins A0, A1, and A2, aligned with the top, middle, and bottom sensors. These values are displayed on the Serial Monitor and can be represented graphically using the Serial Plotter.

Experimental Purpose: The primary objective of this experiment was to evaluate the prototype of a sensing fabric collar's efficacy in monitoring and differentiating between swallowing and non-swallowing behaviors. Participants executed a range of swallowing and non-swallowing tasks while wearing the fabric sensor integrated collar alongside EMG electrodes. The EMG data, capturing electrical activity of specific swallowing-related muscles, served as a benchmark. This reference was crucial in assessing whether the signals obtained from the fabric sensor mirrored real-time swallowing events or exhibited congruent behavior to the EMG readings. By comparing these datasets, it was aimed to validate the reliability and accuracy of the wearable device in monitoring and categorizing swallowing actions.

Participant Recruitment: The study involved a total of 12 participants, including an equal amount of male and female participants. The recruitment process was conducted using multiple channels, such as email invitations, word of mouth, and announcements made by teachers or professors during class sessions.

Inclusion and Exclusion Criteria: To ensure a consistent and reliable evaluation of the wearable fabric collar system, specific inclusion and exclusion criteria were established. The 6 males and 6 females were aged between 18 and 33, with a requirement of under the age 45. All participants were required to be healthy, with no history of swallowing disorders or any medical condition that could potentially affect the swallowing process. This criterion helped to obtain baseline data on healthy swallowing performance, which can be used as a reference for future studies involving patients with dysphagia.

Demographics: The study population consisted of a diverse group of individuals, including students and full-time workers, in order to capture the variability in the swallowing performance among different lifestyles and daily activities. The age range of 18-33 was selected to focus on a young adult population that could provide valuable insights into normal swallowing physiology, serving as a foundation for future research targeting older adults or individuals with specific health conditions.

Ethical Considerations: The participants were informed about the purpose of the study, the procedure, and their rights as study subjects, including the right to withdraw from the study at any time without any consequences. Informed consent was obtained from all participants before their involvement in the trials.

Experimental Setup: The participants were seated upright while wearing the wearable collar; equipped with both the three strain sensors and EMG electrodes. The collar was carefully placed around the neck, ensuring that the laryngeal prominence was positioned directly on the middle sensor. The other two sensors were positioned above and below the laryngeal prominence to accommodate for movements associated with swallowing tasks. In parallel, EMG electrodes were taped below the chin bilaterally 1 cm. from midline and on the forehead as a ground (green), enabling the concurrent measurement of muscle activity during swallowing and non-swallowing tasks.

Experimental Procedure: 1) Before the clinical testing, the participants will complete a pre-test survey to assess their overall health and physical ability, focusing on their swallowing function. 2) Ensure the hardware and data acquisition program are ready to go. 3) Fit the collar and EMG electrodes onto the participant, ensuring the proper positioning of the sensors. 4) Start the data acquisition program and instruct the participant to remain still for approximately 3 seconds to establish a baseline reading. 5) Have the participant perform the specified task one by one. 6) Once the action is completed, record data for another 3 seconds. 7) Stop the data acquisition program and save the collected data as a CSV file. 8) Repeat the process for each task, performing three trials per action until all data have been collected. 9) After the data collection, the participant is asked to complete a comfortability survey based on a numbering rating scale.

TABLE 9
Task List
Tasks List

| Swallowing Actions | Non-Swallowing Actions |
|---|---|
| 20 ml water | Chin Tuck |
| 5 ml water | Cough |
| Saliva Swallow | Head Rotation to the Left |
| Sip Water Through Straw | Inhale and Exhale |
| Sip Water with Effortful | Say ‘Ah’ |
| Swallow | |

Rationale for EMG Electrode Placement: EMG electrodes were placed below the chin to measure the activity of the submental muscles, primarily a group of suprahyoid muscles that contribute to the complex swallowing process. The suprahyoid muscles play an essential role in the swallowing process. During the pharyngeal phase of swallowing, these muscles contract to elevate the hyoid bone and the larynx. This movement assists in the opening of the upper esophageal sphincter, allowing the food bolus to pass from the pharynx into the esophagus. Simultaneously, it helps close the epiglottis, a flap of cartilage located at the base of the tongue, to prevent aspiration of the food or liquid into the airways. By measuring the electrical activity of these muscles, it is possible to differentiate between swallowing and non-swallowing events, providing valuable information for the validation of the wearable collar system.

Data Collection Process: Parameters included: The primary parameters included during the data collection process were age, sex, and health status of the participants. Prior to the experiment, a pre-test survey was conducted to assess the participants' overall health and physical ability, particularly focusing on their swallowing function. This survey helped to ensure that only healthy individuals with no history of swallowing disorders were included in the study. Additionally, after the experiment, participants were asked to complete a comfortability survey to evaluate their experience with the wearable collar and provide valuable feedback for potential design improvements.

Duration of Data Collection: Each participant completed 12 tasks (swallowing and non-swallowing) for three trials each. The total data collection time for each participant, therefore, was the time taken to complete these 36 actions (12 tasks×3 trials), plus the time required for sensor calibration and resting periods between trials. The exact duration of data collection for each participant varied depending on individual performance and adherence to the testing procedure. Nevertheless, the duration (~1 hr.) was kept reasonably consistent across all subjects to ensure comparability of the data.

Storage and Organization of Data: The data collected during the experiments were stored as CSV files, with each file corresponding to a specific trial. To facilitate easy access and organization, the files were stored in separate folders for each participant, identified by their unique subject number. This storage and organization system facilitated efficient data retrieval, allowing for convenient access and analysis during the subsequent data processing and analysis phase of the study.

Preprocessing Steps: Noise Reduction: One of the first preprocessing steps involved isolating individual swallowing and non-swallowing events by aligning the trials for each subject. The action isolation was achieved by examining the output CSV files generated by the data acquisition program and aligning each action's trials for every participant. By focusing solely on the action signals, extraneous noise from the sensors was effectively eliminated.

Savitzky-Golay Filtering: To further enhance the quality of the collected data, a Savitzky-Golay filter was applied to the strain sensor signals. This filtering technique smooths the data by fitting successive sub-sets of adjacent data points with a low-degree polynomial using the method of least squares. For this study, a window size of 21 data points and a polynomial order of 9 were employed. The Savitzky-Golay filter is particularly suitable for this application as it preserves the high-frequency content of the data, ensuring that the fine details of the strain signals, critical for differentiating between swallowing and non-swallowing events, are retained.

The window size specifies the number of points within the moving window, while the polynomial order dictates the degree of the fitted polynomial. The choice of Savitzky-Golay filtering is motivated by its ability to preserve higher moments in the data and its effectiveness in reducing high-frequency noise while retaining essential features of the underlying signal.

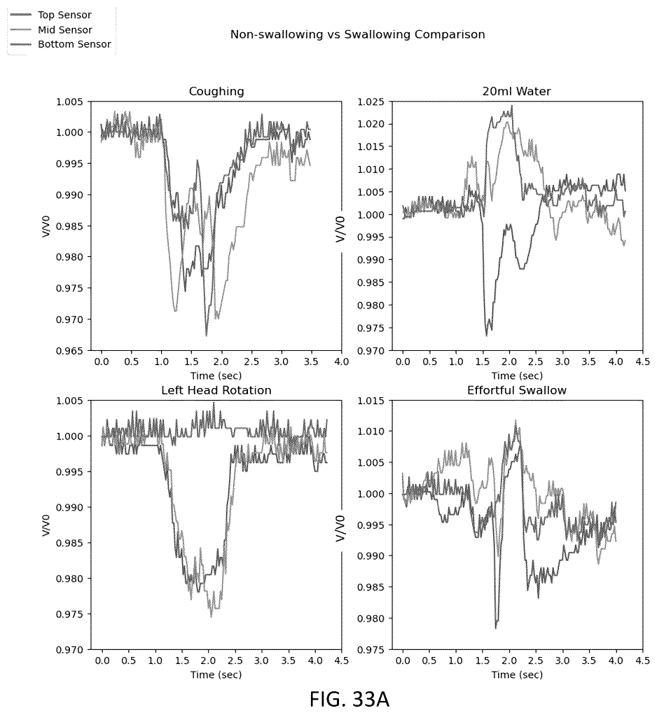

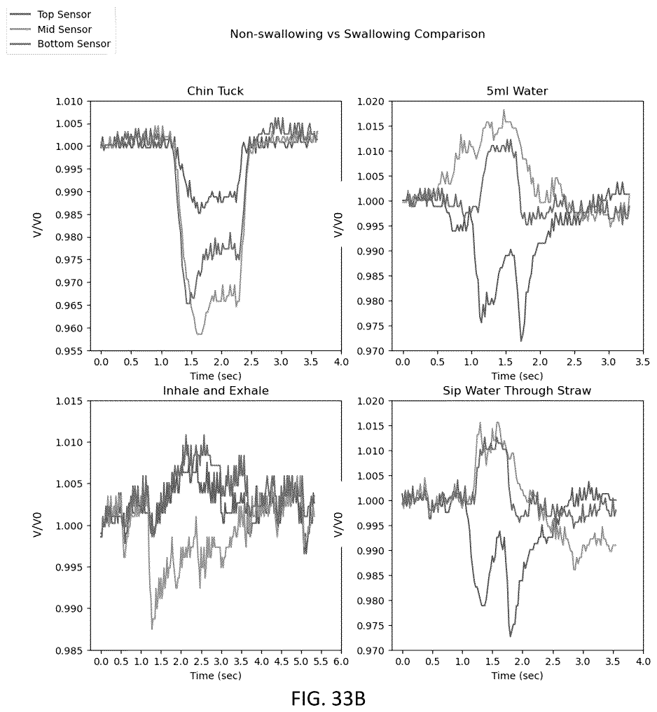

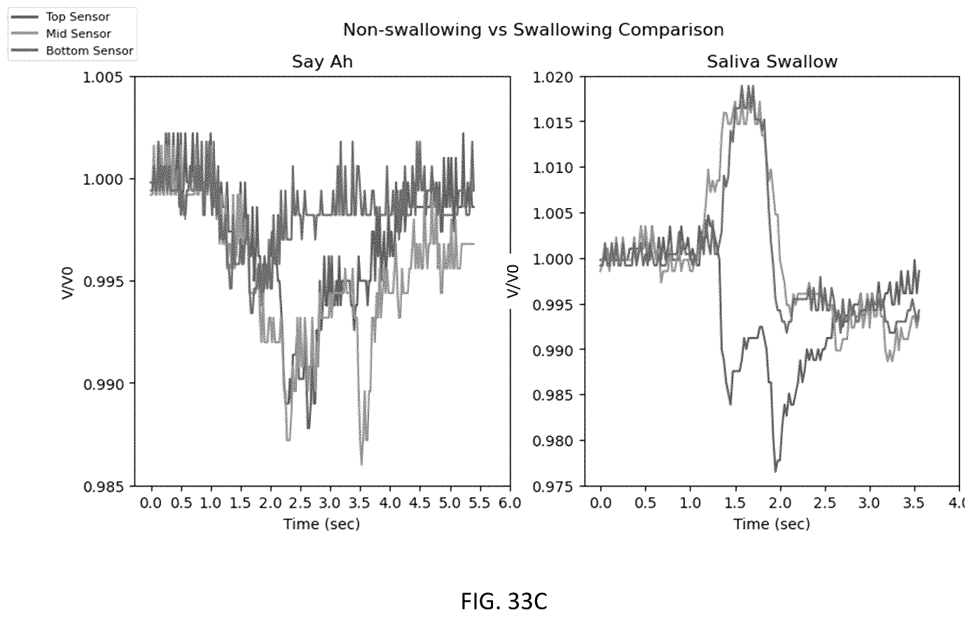

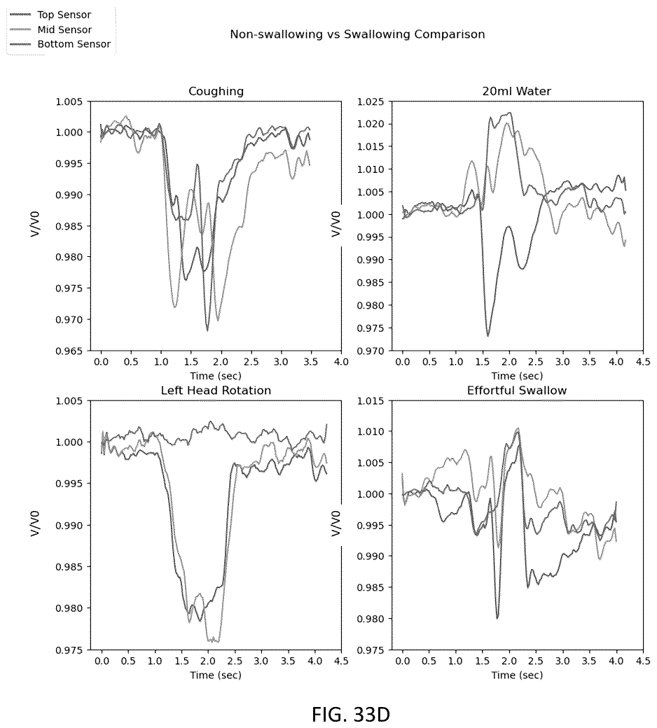

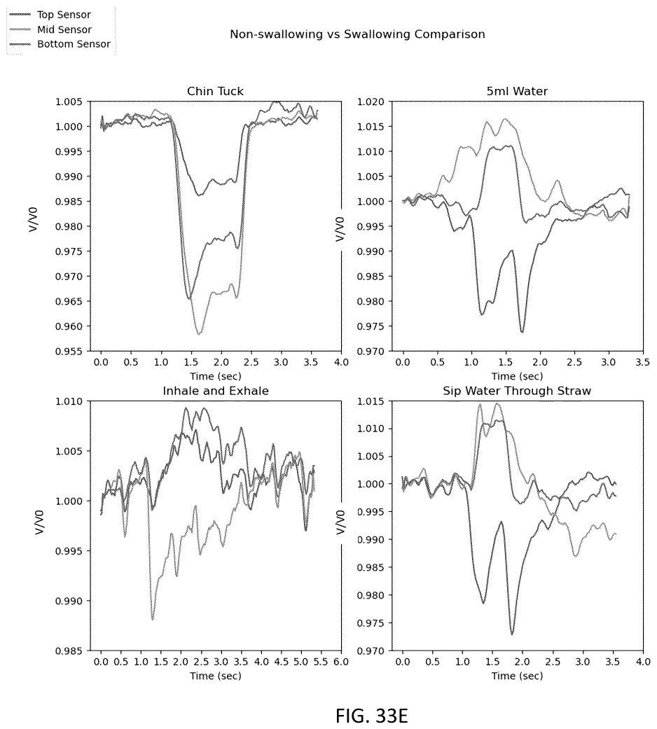

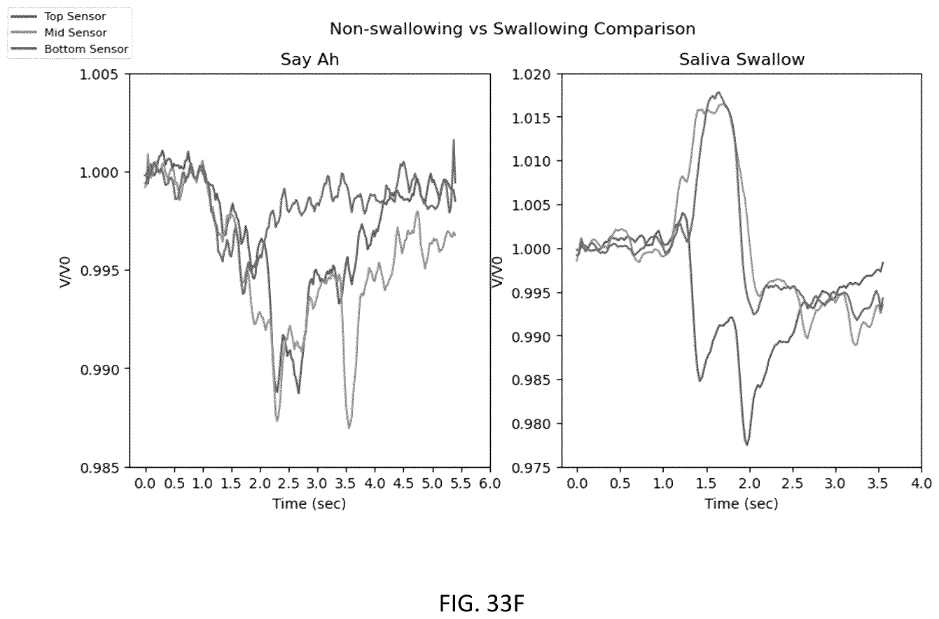

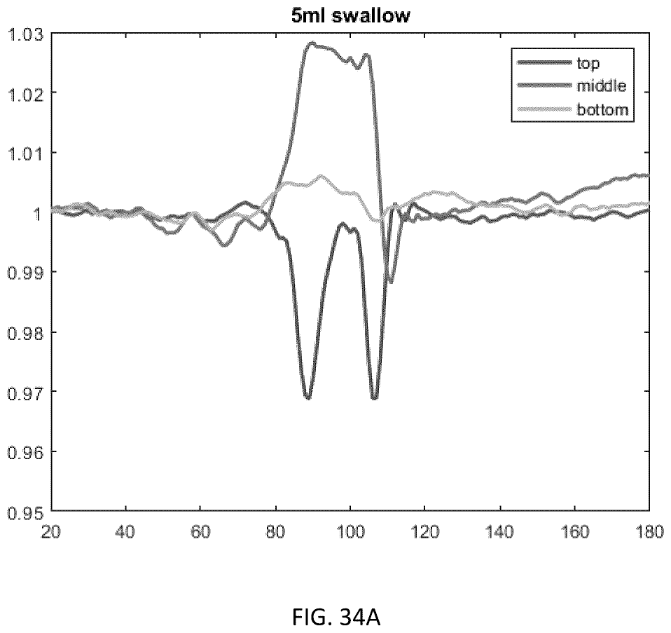

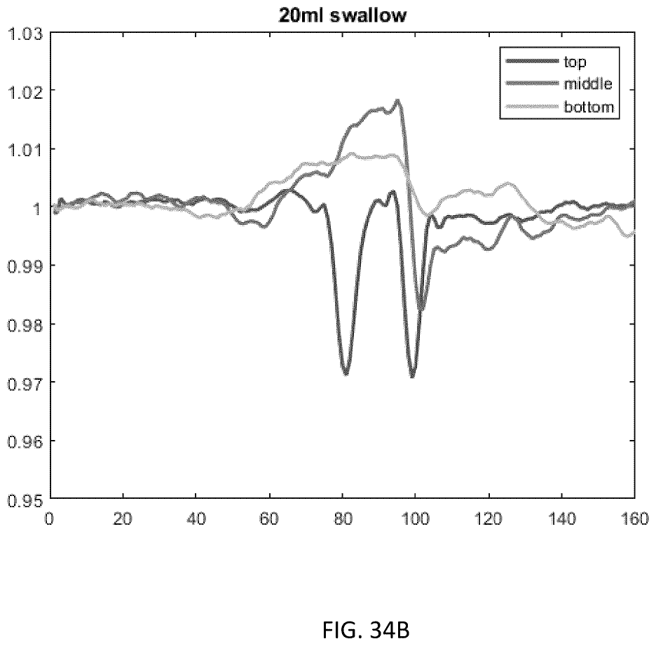

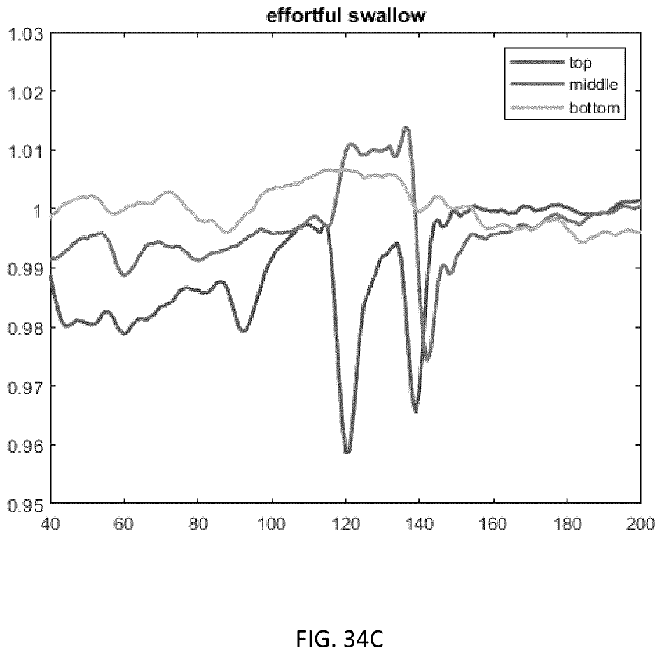

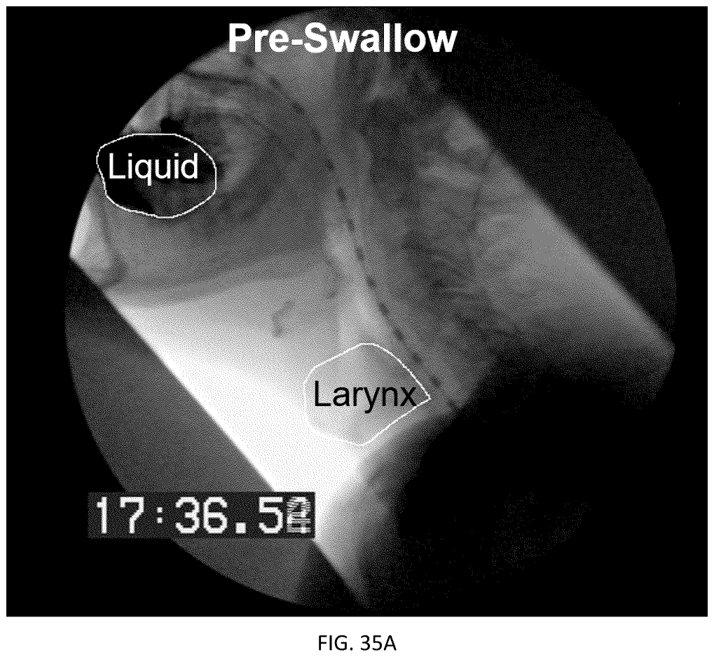

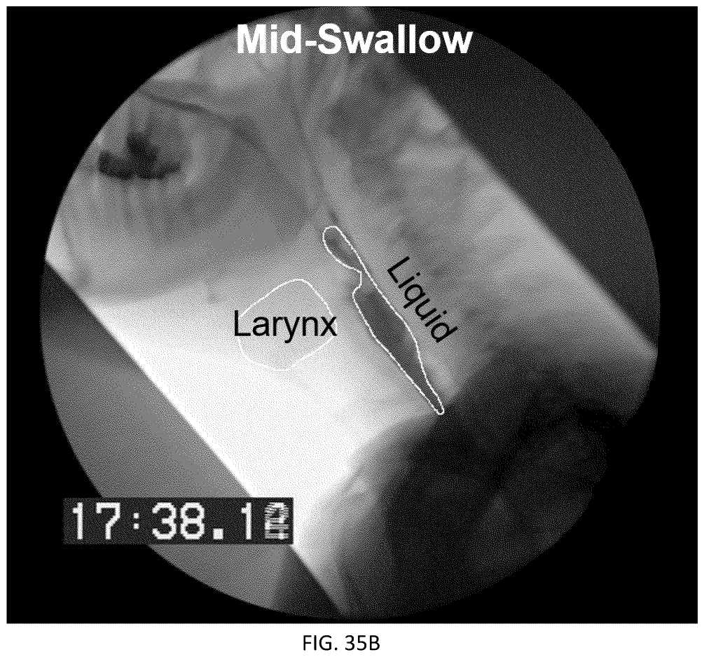

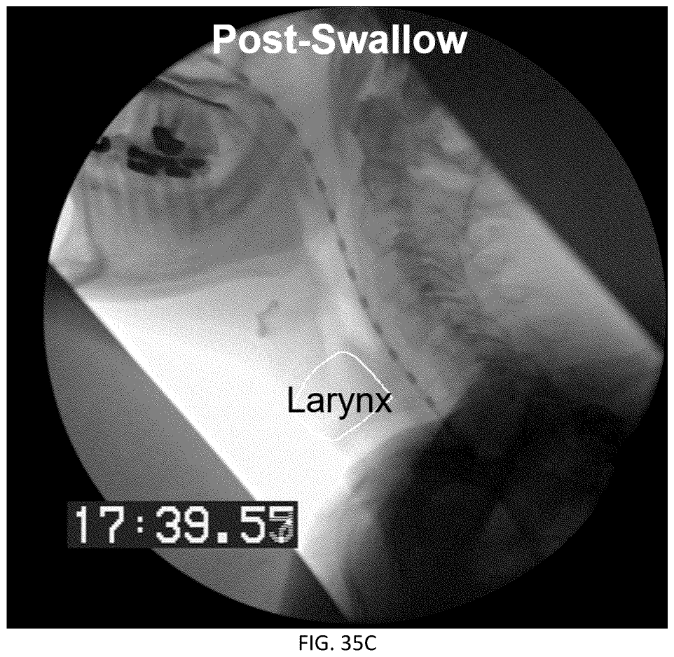

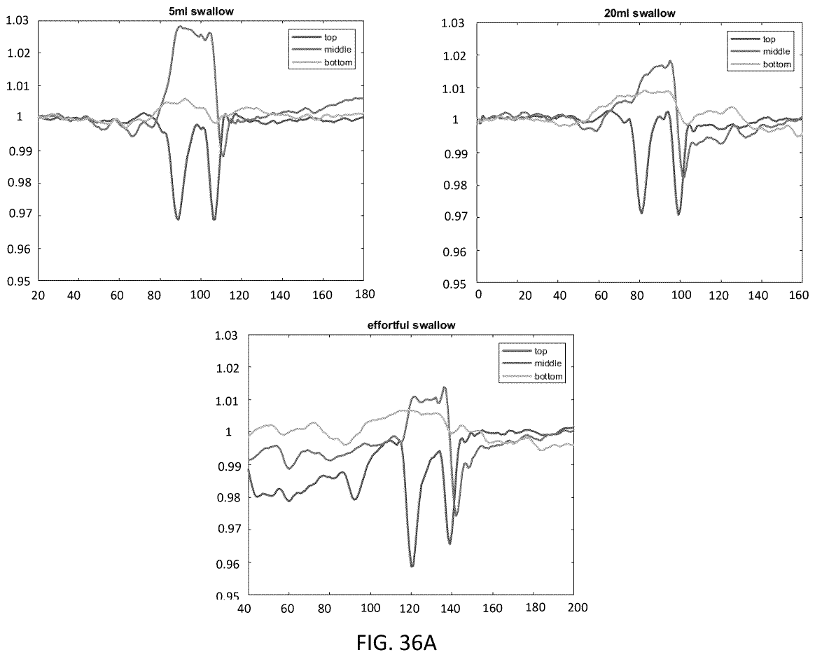

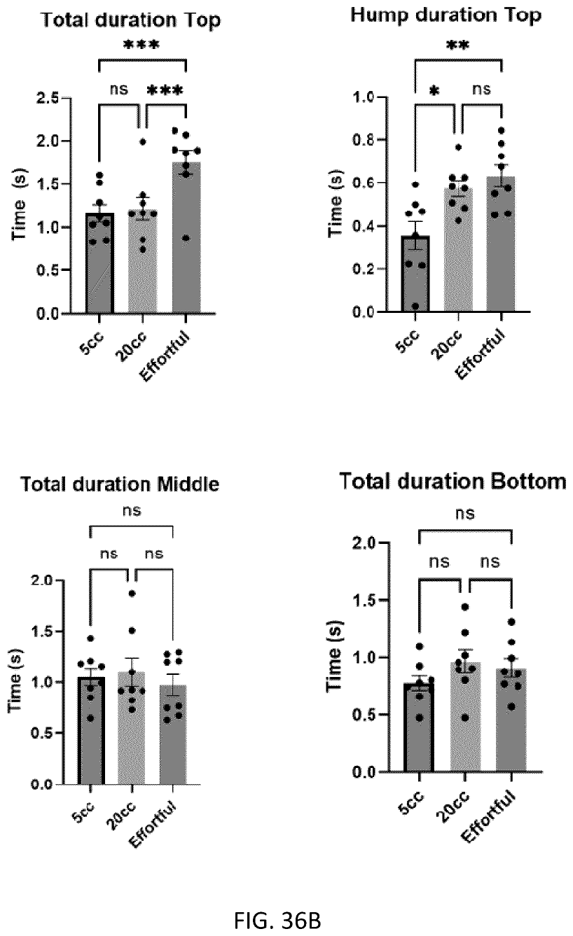

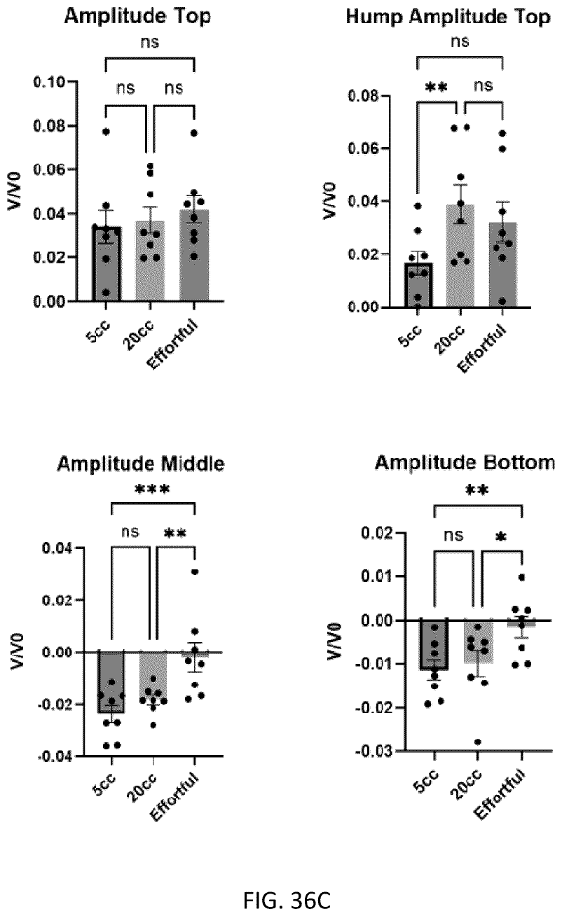

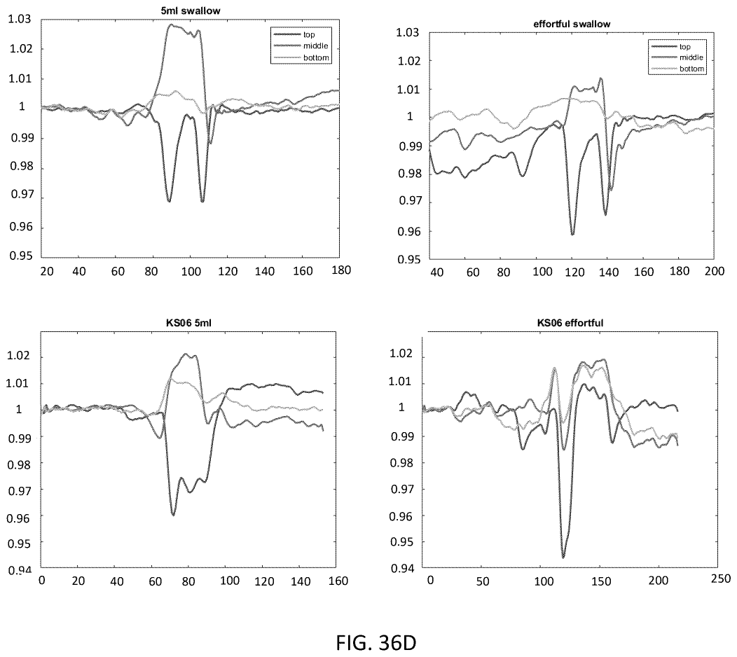

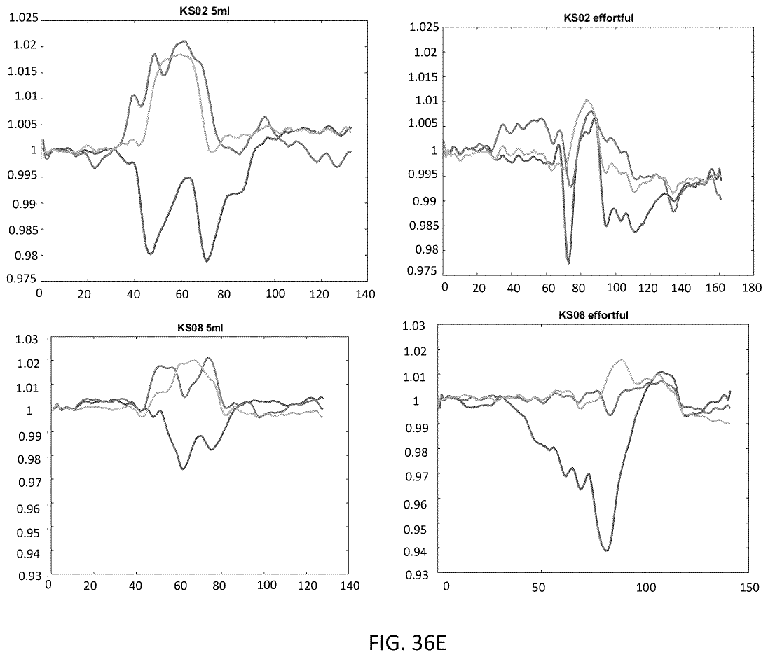

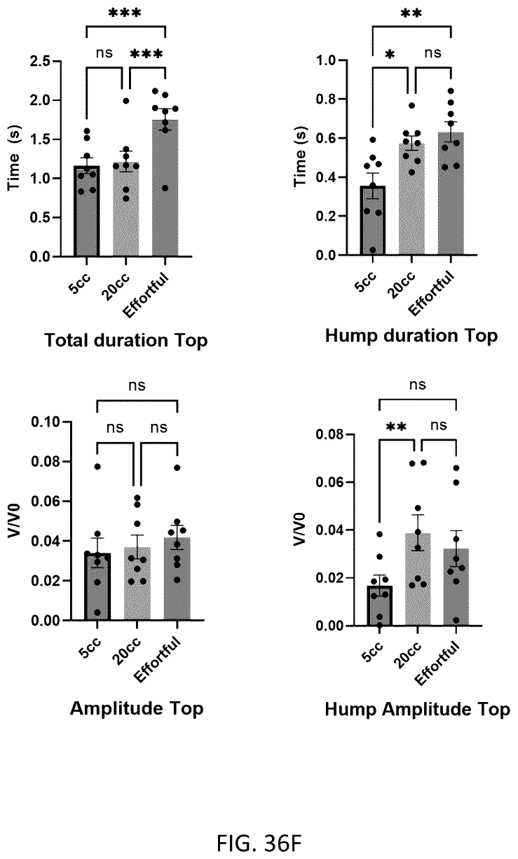

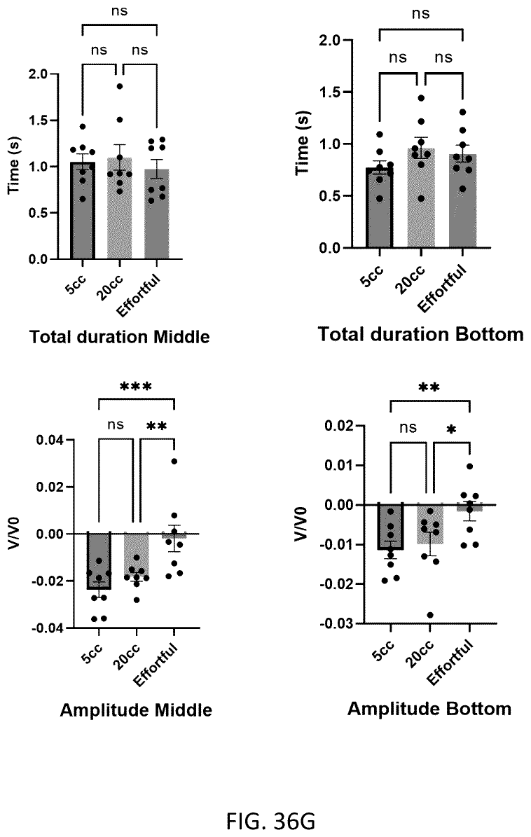

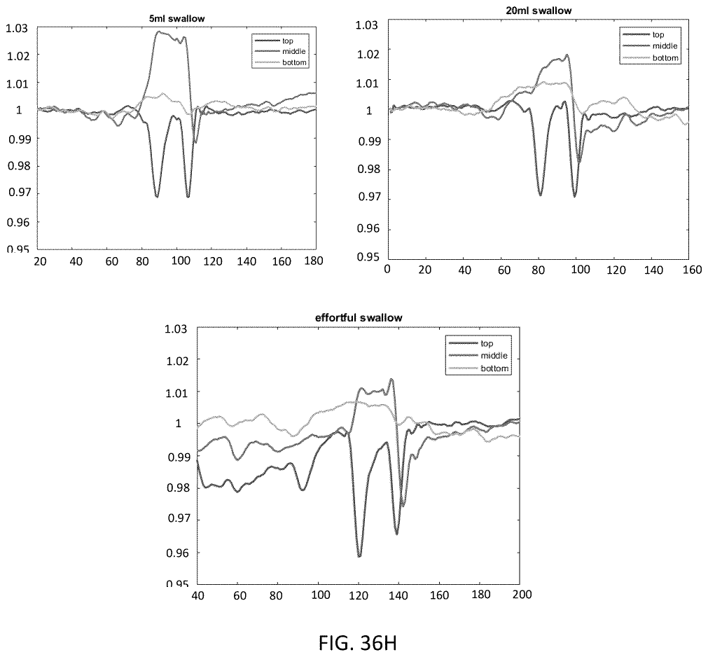

Raw and filtered sensor data for each task performed by the subjects are included in FIG. 33A through FIG. 33F.

Data Segmentation: Once the raw data was filtered and smoothed, it was segmented into individual trials corresponding to the 12 tasks performed by each participant. To enhance the precision of this segmentation, each participant was instructed to remain static for 3 seconds prior to and following each task. This provision of ‘quiet’ periods ensured a clear distinction between the trials, allowing for the precise identification of the start and end times of each task.

Machine Learning Model: In this study, several machine learning techniques were employed to analyze, classify, and predict the swallowing patterns captured by the wearable sensing device. These techniques were implemented using MATLAB's classification learner app, which provides a user-friendly interface for training, evaluating, and comparing different machine learning models. The selected techniques include: Decision Trees: Decision trees are a popular machine learning method used for classification and regression tasks. They work by recursively partitioning the input space and defining a set of decision rules based on the most discriminative features of the data.

Support Vector Machines (SVM): SVMs are a powerful class of supervised learning algorithms that can handle both linear and nonlinear classification problems. They aim to find the optimal decision hyperplane that best separates the data into different classes; with the maximum margin, by mapping the feature space into a higher-dimensional space using kernel functions.

k-Nearest Neighbors (k-NN): k-NN is a simple and effective instance-based learning algorithm that can be employed for classification and regression tasks. It works by finding the k training instances closest to a new input and assigning the most common class label among the k nearest neighbors.

Ensemble Methods: Ensemble methods are a family of machine learning techniques that combine multiple base models to improve classification performance. These methods work by exploiting the complementary strengths of different algorithms and reducing the likelihood of overfitting. In this study, ensemble methods, such as Bagging, Boosting, and Random Forests, were employed to enhance the classification performance of the individual models and achieve a more robust and accurate pattern classification.

Artificial Neural Networks (ANN): ANNs are a class of machine learning models inspired by the structure and function of biological neural networks. They consist of interconnected nodes or neurons that process and transmit information through weighted connections.

In summary, the machine learning techniques employed in this study were selected based on their ability to handle complex and high-dimensional data, as well as their suitability for classification tasks. By leveraging the capabilities of these algorithms, the study aimed to develop accurate and robust models for analyzing, classifying, and predicting swallowing patterns.

Feature Extracted: In the context of swallowing assessment using the wearable sensing device, extracting relevant features from the collected data is essential for understanding and interpreting the swallowing and non-swallowing actions of patients. The signals from the three strategically placed sensors on the wearable fabric collar emulate the traditional three-finger test used in clinical swallow examinations, which provides essential information about the dynamics of the swallowing process. This sub-section delves into the feature extraction methods employed in this study and their mathematical foundations.

Statistical Features: Statistical features play a crucial role in comprehensively understanding the characteristics of sensor signals during the swallowing process. Derived from fundamental statistics, these features offer insights into the distribution and variability of the data. The standard deviation, for instance, quantifies the dispersion of the signal around its mean, indicating the degree of fluctuation during a swallow. A pronounced deviation may correspond to the complex muscular actions in a swallowing event. Skewness provides a measure of the asymmetry of the signal distribution, while kurtosis evaluates the sharpness of its peak. Both skewness and kurtosis can be vital in pinpointing deviations from a typical swallow pattern, aiding in early detection of anomalies.

Skewness: In the context of a biosignal wave, skewness can provide insights into the shape and symmetry of the underlying distribution of the signal's amplitude values. Disclosed herein is a mathematical overview of calculating skewness for a given dataset:
1. Calculate the mean (u) of the biosignal wave.
2. Calculate the standard deviation (o) of the biosignal wave.
3. Compute the cube of the deviations from the mean:

$${( {y}_{i} - μ )}^{3} \qquad Equation 9$$

4. Calculate the mean of the cubed deviations:

$$E ( {( {y}_{i} - μ )}^{3} ) = \frac{\sum {( {y}_{i} - μ )}^{3}}{N} \qquad Equation 10$$

5. Calculate the skewness (y) of the dataset:

$$γ = \frac{E ( {( {y}_{i} - μ )}^{3} )}{{σ}^{3}} \qquad Equation 11$$

Skewness is a measure of the asymmetry of a distribution. Skewness can provide insights into the shape and symmetry of the underlying distribution of the signal's amplitude values. A positive skewness indicates that the distribution has a longer tail on the right side, while a negative skewness indicates a longer tail on the left side. A skewness value close to 0 suggests that the distribution is relatively symmetric.

Kurtosis: Kurtosis is a statistical measure that describes the “tailedness” or the shape of the probability distribution of a dataset. In the context of a biosignal wave, kurtosis provides insights into the shape, specifically the peakedness or flatness of the distribution of the signal's amplitude values. Disclosed herein is a mathematical overview of calculating kurtosis for a given dataset:
1. Compute the fourth power of the deviations from the mean:

$${( {y}_{i} - μ )}^{4} \qquad Equation 12$$

where yᵢ are the individual data points and μ is the mean.
2. Calculate the mean of the fourth power deviations:

$$E ( {( {y}_{i} - μ )}^{4} ) = \frac{( \sum {( {y}_{i} - μ )}^{4} )}{N} \qquad Equation 13$$

where Σ is the sum of the data points, and N is the total number of data points.
3. Calculate the kurtosis (k) of the dataset:

$$k = \frac{E ( {( {y}_{i} - μ )}^{4} )}{{σ}^{4}} \qquad Equation 14$$

where σ is the standard deviation.

A higher kurtosis value indicates a more peaked distribution with heavier tails, while a lower kurtosis value suggests a flatter distribution with thinner tails. A kurtosis value of 3 indicates a normal distribution (mesokurtic). If the kurtosis is greater than 3, the distribution is considered leptokurtic (heavy-tailed), and if it is less than 3, the distribution is platykurtic (light-tailed).

Time Domain Features: In signal processing for swallow detection, the time domain features serve as foundational indicators that capture the temporal progression of swallowing events. These features are directly derived from the sensor signals as they unfold over time. Peak detection, a pivotal component, focuses on identifying significant elevations or depressions in the signal, representing crucial moments like the initiation or culmination of a swallow. The provided plot, for instance, vividly showcases a trough followed by a peak between 1 to 2.5 seconds, suggesting a swallow event. Moreover, determining the duration of these peaks offers invaluable data about the swallow's entirety, revealing whether the event occurred smoothly or if interruptions were present, thus ensuring a holistic understanding of the swallowing mechanism.

Frequency Domain Features: Frequency domain features delve into the inherent spectral characteristics of these biosignals waves. One of the pivotal features in this domain is the bandwidth, which effectively encapsulates the range of frequencies encapsulating most of the signal's power. By employing power spectral density (PSD) estimation through the periodogram method, the bandwidth of a biosignal wave can be precisely determined. This bandwidth, in turn, provides a nuanced view into the frequency spread of the signal. Understanding this spread is vital, as distinct swallowing events might resonate at different frequency ranges, aiding in distinguishing between various patterns or abnormalities in the swallowing mechanism.

Disclosed herein is a mathematical overview of calculating the bandwidth for a biosignal wave:
1. Calculate the periodogram of the trial_data to obtain the power spectral density (PSD) estimation. The periodogram is computed using the squared magnitude of the discrete Fourier transform (DFT) of the signal divided by the number of samples (N):

$$P x x ( f ) = ( 1 / N ) * {\vert DFT ( trial\_data ) \vert}^{2} \qquad Equation 28$$

2. Compute the spectral centroid of the PSD:

$${Spectral}_{Centroid} = \frac{\sum ( {F}_{i} * {Pxx}_{i} )}{\sum ( {Pxx}_{i} )} \qquad Equation 29$$

Here, Pxx represents the PSD, and F denotes the corresponding frequencies. Calculate the bandwidth of the signal:

$$Bandwidth = \sqrt{\frac{\sum ( {( {F}_{i} - {Spectral}_{Centroid} )}^{2} * {Pxx}_{i} )}{\sum ( {Pxx}_{i} )}} \qquad Equation 30$$

Upon extracting these features, they can be fed into a machine learning algorithm for training. The Savitzky-Golay filtering ensures that the high-frequency content critical for distinguishing events is retained, making these features robust. By creating a feature vector comprising these values and others derived from the signal, we can train the disclosed model to differentiate between a healthy swallow and non-swallow events with precision, mimicking the expertise of a clinician in a quantitative manner.

Hyperparameter tuning process: Developing a high-performing machine learning model necessitates the careful tuning of hyperparameters. This process involves determining the optimal combination of hyperparameters, which can significantly influence the model's performance. In this study, the k-Nearest Neighbors (KNN) algorithm was employed for classification, and its hyperparameters were carefully tuned to balance the classification accuracy and computational complexity.

The KNN algorithm's hyperparameters include the following:
Number of Neighbors (k): This hyperparameter determines the number of neighboring data points considered when making a classification decision. A smaller value of k results in a more flexible model, which can capture intricate patterns in the data but may be prone to overfitting. Conversely, a larger value of k yields a smoother decision boundary, potentially increasing the model's generalization ability but potentially sacrificing sensitivity to local patterns. In this study, a value of k=1 was chosen after evaluating different values of k using cross-validation. The choice of k=1 provided the best performance in accuracy, precision, and F-1 score ensuring accurate classification while minimizing the risk of overfitting.
Distance Metric: The distance metric is used to compute the distance between data points in the feature space. Common distance metrics include Euclidean, Manhattan, and Mahalanobis distances. The choice of distance metric can influence the model's performance and should be selected based on the data characteristics and the problem's nature. In this study, the Euclidean distance metric was employed because it is invariant to rotation, making it suitable for detecting differences in feature space across various orientations. Furthermore, the Euclidean distance is computationally efficient and provides satisfactory performance in practice.
Distance Weight: This hyperparameter determines the weighting scheme applied to the neighbors' votes. When using equal weighting, each neighbor contributes equally to the classification decision. Alternatively, distance weighting schemes, such as inverse distance or Gaussian weighting, assign higher importance to closer neighbors. In this study, equal weighting was chosen after comparing the performance of various weighting schemes. Equal weighting yielded similar classification accuracy to more complex weighting schemes while requiring fewer computational resources, making it a suitable choice for this application.
Standardize Data: Standardizing the data involves scaling the feature values to have zero mean and unit variance. This process ensures that all features contribute equally to the distance calculation, preventing features with larger magnitudes from dominating the classification decision. In this study, the data were standardized to account for potential differences in feature magnitudes and measurement units, leading to improved model performance.

The KNN classifier was rigorously tuned by training and evaluating it on a variety of hyperparameter combinations using 10-fold cross-validation, which means that 90% of the data was used for training, and 10% was set aside for testing in each iteration. This systematic method, known as grid search or parameter sweep, is instrumental for optimal hyperparameter selection.

A prominent feature of the disclosed tuning process was the variation in the number of neighbors (K) in the KNN algorithm. The classifier performance metrics, such as Accuracy, Precision, Sensitivity, Specificity, and F1-score, were observed to decrease as the value of K increased from 1 to 10, indicating that a lower value of K yielded better results for the specific application (see FIG. 30B).

The selection of distance metric also played a substantial role in the disclosed model performance. The Euclidean distance metric led to the best performance, with Accuracy of 75.6%, Precision of 72.4%, Sensitivity of 81.5%, Specificity of 74.4%, and F1-score of 76.7%. Cosine and Minkowski (cubic) metrics showed slightly lower performance, indicating that the choice of distance metric has a weaker influence on the model's outcomes compared to the number of neighbors and model type (FIG. 30C).

The disclosed classifier's model type was another critical hyperparameter, with Fine KNN achieving the highest scores across all performance metrics. Cubic SVM and Medium Neural Network configurations, though still adequate, performed less well (FIG. 30A).

Overall, the carefully selected hyperparameter combinations led to a model with high classification accuracy and acceptable computational complexity. This result illustrates the potential of the disclosed wearable sensing device and algorithm for differentiating swallowing and non-swallowing actions in healthy subjects.

Validation process: The MATLAB Classification Learner app facilitates supervised machine learning processes by allowing users to interactively train, validate, and compare multiple classification models using statistical and machine learning algorithms. The intuitive interface aids in feature selection, model tuning, and visualization, streamlining the workflow for both novice and experienced users.

To evaluate the performance of the machine learning model, various metrics can be calculated from a confusion matrix. A confusion matrix is a table that shows the true positive (TP), false positive (FP), true negative (TN), and false negative (FN) values for a binary classification problem. The confusion matrix can be extended to multi-class classification problems as well.

Here's an explanation of how to calculate various performance metrics from a confusion matrix:
Accuracy: It is the proportion of correctly classified instances out of the total instances. Formula: Accuracy=(TP+TN)/(TP+TN+FP+FN)
Sensitivity (Recall): It is the proportion of actual positive instances that were predicted as positive. Formula: Sensitivity=TP/(TP+FN)
Specificity: It is the proportion of actual negative instances that were predicted as negative. Formula: Specificity=TN/(TN+FP)
Precision: It is the proportion of predicted positive instances that were actually positive. Formula: Precision=TP/(TP+FP)
F1-score: It is the harmonic means of precision and recall, which provides a balance between them. Formula: F1-score=2*(Precision*Sensitivity)/(Precision+Sensitivity)

TABLE 10
Performance Metrics

| | | | Medium |
|---|---|---|---|
| | Fine | Cubic | Neural |
| Model | KNN | SVM | Network |
| Accuracy | 75.60% | 67.50% | 56.30% |
| Precision | 72.40% | 59.10% | 48.20% |
| Sensitivity | 81.50% | 68.40% | 59.90% |
| Specificity | 74.40% | 68.00% | 55.10% |
| F1-score | 76.70% | 63.40% | 53.40% |

The performance assessment of the disclosed device, grounded in the extracted features and processed signals, pivoted on three machine learning models: Fine KNN, Cubic SVM, and Medium Neural Network. Utilizing the MATLAB Classification Learner app ensured a robust supervised learning environment, optimizing feature selection, model tuning, and comparative analysis.

Central to the evaluation was the confusion matrix, detailing true positives (TP), false positives (FP), true negatives (TN), and false negatives (FN). Through this, key metrics—accuracy, sensitivity, specificity, precision, and F1-score—were derived.

Starting with accuracy, which denotes the ratio of correctly classified instances over total instances, Fine KNN emerged superior with an accuracy of 75.6%. In comparison, Cubic SVM and Medium Neural Network trailed with 67.5% and 56.3%, respectively. This suggests that Fine KNN, among the three models, most reliably differentiates between swallowing and non-swallowing events.

Sensitivity, or recall, measures the capability of a model to correctly identify actual positive events. A high sensitivity ensures that true swallowing events are seldom missed. Fine KNN led the trio with a sensitivity of 81.5%, indicating its efficacy in true positive classification. Cubic SVM followed with 68.4%, and Medium Neural Network lagged at 59.9%.

Specificity, on the other hand, gauges a model's proficiency in correctly identifying actual negative instances. Fine KNN recorded a specificity of 74.4%, outclassing Cubic SVM and Medium Neural Network, which registered 68.0% and 55.1% respectively. This implies that Fine KNN demonstrates a balanced performance, adeptly distinguishing both positive and negative instances.

Precision determines the portion of predicted positive events that were actual positives. It is pivotal in ensuring that false alarms are minimized. Here, Fine KNN again dominated with 72.4%, while Cubic SVM and Medium Neural Network recorded 59.1% and 48.2%. A higher precision in Fine KNN indicates fewer false swallowing event detections.

Lastly, the F1-score, which balances precision and sensitivity, was used to assess overall model efficiency. With an F1-score of 76.7%, Fine KNN solidified its superiority. In contrast, Cubic SVM posted a score of 63.4%, and Medium Neural Network trailed with 53.4%.

In synthesis, the Fine KNN model stands out as the most competent model across all evaluated metrics. Its consistent performance in accuracy, sensitivity, specificity, and F1-score underscore its robustness in classifying swallowing events using the features extracted from the disclosed device. Cubic SVM occupies a median position, offering moderate reliability. Meanwhile, the Medium Neural Network, despite its intrinsic capacity for complex pattern recognition, appears less suited for this application, at least with the current architecture and training parameters.

In conclusion, anchored in the feature extraction process and evaluated metrics, Fine KNN emerges as the optimal choice for real-time swallow monitoring using the disclosed device. Future endeavors might explore further refining the KNN parameters or enhancing the neural network's architecture for improved outcomes.

The incorporation of machine learning applications into wearable devices, particularly for the assessment of dysphagia, is a burgeoning field in healthcare technology. Drawing from the literature, various innovative methods ranging from fabric-based sensors [Zhang, R., et al. A generic sensor fabric for multi-modal swallowing sensing in regular upper-body shirts. in Proceedings of the 2016 ACM International Symposium on Wearable Computers. 2016] to epidermal electronics [Constantinescu, G., et al., Epidermal electronics for electromyography: An application to swallowing therapy. Medical engineering & physics, 2016. 38(8): p. 807-812] have been proposed and tested for their efficacy in detecting and assessing swallowing events. This research's wearable device stands as a testament to the advancements in this realm, emphasizing the utilization of accurate machine learning models to enhance its precision.

Performance metrics are paramount in determining the efficacy of any machine learning application. In this context, the device in question employed three different machine learning models: Fine KNN, Cubic SVM, and Medium Neural Network. Analyzing the metrics, Fine KNN outperformed the other models, yielding an accuracy of 75.6%, sensitivity of 81.5%, and an F1-score of 76.7%. This surpasses the multi-modal swallowing detection capability reported by Zhang et al., where swallows were identified with a 75% overlap with expert annotations using fabric-based sensors. Moreover, while Lee et al. achieved a remarkable average successful response rate upwards of 94% using soft electronic systems [Lee, Y., et al., Soft electronics enabled ergonomic human-computer interaction for swallowing training. Scientific Reports, 2017. 7(1): p. 46697], the nature of their study leaned more towards ergonomic human-computer interactions, particularly for swallowing training, and was not solely focused on dysphagia assessment.

The adaptability feature of the epidermal sEMG patches discussed by Constantinescu et al. bears resemblance to the flexibility of this research's wearable device, tailored to the unique needs of both pediatric and adult populations. However, the device in this study demonstrates a more comprehensive approach, encompassing advanced machine learning models to ensure precise dysphagia assessment.

Hashimoto et al.'s non-invasive Swallow Tracking System [Hashimoto, H., et al., Non-invasive quantification of human swallowing using a simple motion tracking system. Scientific reports, 2018. 8(1): p. 5095] and Shieh et al.'s device utilizing a force-sensing resistor [Shieh, W.-Y., C.-M. Wang, and C.-S. Chang, Development of a portable non-invasive swallowing and respiration assessment device. Sensors, 2015. 15(6): p. 12428-12453] further substantiate the potential of non-invasive methodologies. Yet, their focus primarily remains on tracking and documenting motion during swallowing, whereas the disclosed research integrates the power of machine learning to offer a more detailed and accurate analysis.

The application of wearable sensors, especially in the realm of swallowing disorders, has seen remarkable developments. From Kim et al.'s submental sensor patch [Kim, M. K., et al., Flexible submental sensor patch with remote monitoring controls for management of oropharyngeal swallowing disorders. Science advances, 2019. 5(12): p. eaay3210] to the piezoelectric sensor-based necklace for monitoring eating habits by Kalantarian et al. [Kalantarian, H., et al., Monitoring eating habits using a piezoelectric sensor-based necklace. Computers in biology and medicine, 2015. 58: p. 46-55], the versatility and potential of such devices are evident. This research's device builds upon these foundations but stands distinct due to its specialized machine learning feature extraction process, which not only ensures precise detection but also offers valuable insights into dysphagia assessment.

The unique strengths of the device presented in the disclosed study are manifested in its high sensitivity and specificity, coupled with the reliability of machine learning models like Fine KNN. When juxtaposed with Wei et al.'s ultra-sensitive artificial graphene throat [Wei, Y., et al., A wearable skinlike ultra-sensitive artificial graphene throat. ACS nano, 2019. 13(8): p. 8639-8647] and Li et al.'s wearable sensor integrating graphene and cotton [Li, P., et al., A wearable and sensitive graphene-cotton based pressure sensor for human physiological signals monitoring. Scientific reports, 2019. 9(1): p. 14457], the device in the disclosed research does more than just monitoring physiological signals. It provides a comprehensive analysis, making it a powerful tool for both diagnosing and categorizing swallowing disorders.

In summary, while several studies have explored the domains of wearable sensors and machine learning in dysphagia assessment, the device under discussion in this research emerges as a front-runner. Its robust machine learning models, coupled with a precise feature extraction process, position it as a reliable, efficient, and technologically advanced tool in the field of dysphagia assessment. This endeavor not only contributes significantly to the existing body of knowledge but also paves the way for future advancements in non-invasive, accurate, and user-friendly solutions tailored for varied age groups battling swallowing disorders.

The device holds promise in various scenarios. Clinically, it can be utilized during patient consultations, post-operative monitoring, and therapy sessions, acting as a reliable diagnostic tool. Non-clinically, its applicability extends to at-home monitoring and rehabilitation centers, promoting continued patient care outside hospital settings.

Some advantages of the disclosed device is that it is built from accessible components, the device stands as an affordable alternative, making cutting-edge swallowing assessment reachable for a broader audience. This cost-effective approach, combined with its innovative features, paves the way for its widespread adoption, benefiting both patients and healthcare professionals in dysphagia management.

This research has made significant contributions to the understanding and development of wearable sensors for monitoring swallowing. By designing and implementing a wearable sensing device equipped with three strategically placed sensors, the study has demonstrated the feasibility of non-invasive, continuous monitoring of swallowing patterns in real-world settings. This innovative approach offers advantages over traditional clinical assessments, such as videofluoroscopy, which are often limited by invasiveness, radiation exposure, and constrained environments. The development of machine learning algorithms for the classification and analysis of swallowing events represents another important contribution of this research. By identifying key features associated with swallowing biomechanics, the study has demonstrated the potential for data-driven models to classify swallowing events accurately and objectively. This is particularly valuable for healthcare professionals as it provides a quantitative basis for dysphagia assessment, leading to more informed diagnoses and treatment decisions. The disclosed findings have the potential to improve dysphagia diagnosis, treatment, and monitoring in several ways. The wearable sensing device enables continuous and real-time monitoring of swallowing patterns, which could allow for earlier detection of dysphagia and timely intervention. The machine learning algorithms developed in this research could be used to identify subtle changes in swallowing patterns that might otherwise go unnoticed, helping to prevent complications and optimize treatment outcomes. By enabling more accurate and efficient swallowing assessment, the device may help patients receive the appropriate treatment sooner and minimize the risk of complications, such as aspiration pneumonia. Furthermore, by allowing patients to monitor their swallowing patterns at home, the device empowers them to take a more active role in managing their condition and facilitates communication with healthcare providers. For healthcare professionals, the wearable sensing device offers several benefits. By providing objective and quantitative data on swallowing patterns, the device could complement existing assessment methods and enhance the accuracy of dysphagia diagnoses. The machine learning algorithms developed in this research can also help professionals better understand the complex biomechanics of swallowing and identify patterns that may be indicative of underlying pathology. Additionally, the continuous monitoring capabilities of the wearable device can improve the long-term management of patients with dysphagia, enabling healthcare professionals to monitor treatment progress and adjust interventions as needed.

In conclusion, this research has made significant contributions to the development of wearable sensors for dysphagia assessment and has the potential to improve the lives of patients with swallowing disorders. By leveraging innovative sensing technology and advanced machine learning algorithms, this work represents a critical step towards more accurate and efficient dysphagia assessment and care.

In some embodiments, the disclosed knitted strain sensor system comprises features for enabling or improving the fit, comfort, and adjustability of the system. In some embodiments, the system may be formed in multiple sizes, or comprise one or more mechanisms (e.g., a button mechanism) that provides flexibility to accommodate differences in neck size and anatomical variations.

In some embodiments, the knitted strain sensor placement is based on anatomical landmarks, subject, gender, or areas or regions of interest on a subject. In some embodiments, the knitted strain sensor system comprises strain relief or a lightweight enclosure.

The knitted strain sensor systems's real-time swallow monitoring is a significant development, providing transformative advancements in patient care and diagnosis accuracy.

### REFERENCES

The following publications are incorporated herein by reference in their entirety:
Li, Y., Miao, X., Chen, J. Y., Jiang, G., and Liu, Q. Sensing Performance of Knitted Strain Sensor on Two-Dimensional and Three-Dimensional Surfaces. Materials & Design, 197 (2021), 109273. https://doi.org/10.1016/j.matdes.2020.109273.
Bian, H., Tu, P., and Chen, J. Y. Fabrication of All-Cellulose Nanocomposites from Corn Stalk. Journal of the Science of Food and Agriculture, May 9, 2020, https://doi.org/10.1002/jsfa.10476.
Wilson, A. C., Chou, S. F., Lozano, R., Chen, J. Y., and Neuenschwander, P. F. Thermal and Physico-Mechanical Characterizations of Thromboresistant Polyurethane Films. Bioengineering, 2019, 6(3), 69. https://doi.org/10.3390/bioengineering6030069.
Luo, W., Cheng, L., Yuan, C., Wu, Z., Yuan, G., Hou, M., Chen, J. Y., Luo, C., and Li, W. Preparation, characterization and evaluation of cellulose nanocrystal/poly(lactic acid) in situ nanocomposite scaffolds for tissue engineering. International Journal of Biological Macromolecules, 134(2019), 469-479.
R. J. Lozano-Escárcega, M. G. Sánchez-Anguiano, T. Serrano, J. Y. Chen, and I. Gómez. Synthesis of unsaturated polyester resin from waste cellulose and polyethylene terephthalate. Polymer Bulletin, 2018. https://doi.org/10.1007/s00289-018-2576-7.
Luo, W., Li, X., and Chen, J. Y. All-Fabric Flexible Supercapacitor for Energy Storage. Journal of Industrial Textiles, 2018, https://doi.org/10.1177/1528083718804208.
Liu, Y., Edwards, J. V., Prevost, N., Huang, Y. and Chen, J. Y. Physico- and Bio-Activities of Nanoscale Regenerated Cellulose Nonwoven Immobilized with Lysozyme. Materials Science & Engineering: C Materials for Biological Applications, 2018, 91, 389-394. https://doi.org/10.1016/j.msec.2018.05.061.
Chen, J. Y, Sun, L., Negulescu, I. I., and Xu, B. Fabrication and Evaluation of Regenerated Cellulose/Nanoparticle Fibers from Lignocellulosic Biomass. Biomass and Bioenergy, 2017, Vol 101, 1-8. https://doi.org/10.1016/j.biombioe.2017.03.024.
Huang, Y. and Chen, J. Y. All-carbon cord-yarn supercapacitor. Journal of Industrial Textiles, 2017, DOI: 10.1177/1528083717699370.
Liu, Y., Nguyen, A., Allen, A., Zoldan, J., Huang, Y., and Chen, J. Y. Regenerated cellulose micro-nano fiber matrices for transdermal drug release. Materials Science & Engineering: C Materials for Biological Applications, 2017, 74, 485-492. DOI: 10.1016/j.msec.2016.12.048.
Huang, Y., Liu, Y., Zhao, G., and Chen, J. Y. Sustainable activated carbon fiber from wood sawdust by reactivation for high-performance supercapacitors. Journal of Materials Science, 2017, 52, 478-488.
Huang, Y., Peng, L., Liu, Y., Zhao, G., Chen, Y. J., and Yu, G. Biobased nano porous active carbon fibers for high-performance supercapacitors. ACS Appl. Mater. Interfaces 2016, 8, 24, 15205-15215. DOI: 10.1021/acsami.6b02214.
Liu, Y. and Chen, J. Y. Enzyme immobilization on cellulose matrixes. Journal of Bioactive and Compatible Polymers. 2016, 31(6), 553-567. doi: 10.1177/0883911516637377.
Vinogradova, Y. S. and Chen, J. Y. Micron- and Nano-Cellulose Fiber Generated from Ionic Liquid. The Journal of The Textile Institute, 2015. DOI: 10.1080/00405000.2015.1040693.
Sun, L., Chen, J. Y., Jiang, W., and Lynch, V. Crystalline Characteristics of Cellulose Fiber and Film Regenerated from Ionic Liquid Solution. Carbohydrate Polymers, 118(2015), 150-155.
Chen, J. Y., Sun, L., Jiang, W., and Lynch, V. Antimicrobial regenerated cellulose/nano-silver fiber without leaching. Journal of Bioactive and Compatible Polymers, 2015, 30(1), 17-33. DOI: 10.1177/0883911514556960.
Chen, J. Y., Sun, L., Jiang, and Edwards, J. V. Regenerated Cellulose Fiber and Film Immobilized with Lysozyme. Bioceramics Development and Applications, 2014, 4(1): 078. doi: 10.4172/2090-5025.1000078.
Chen, J. Y. and Jiang, N. Fabrication and Characterization of Carbonized and Activated Cotton Nonwovens. Journal of Industrial Textiles, 2014, 43(3), 338-349.
Jiang, W., Sun, L., Hao, A., and Chen, J. Y. Regenerated Cellulose Fibers from Waste Bagasse Using Ionic Liquid. Textile Research Journal, 2011, 81(18), 1949-1958.
Sun, L., Chen, J. Y., Negulescu, I. I., Moore, M. M., and Collier, B. J. Kinetics Modeling of Dynamic Pyrolysis of Bagasse Fibers. Bioresource Technology, 2011, 102, 1951-1958.
Chen, J. Y. Chapter 4 Man-made fibres: regenerated cellulose fibres. in Textiles and fashion: materials, design and technology, Rose Sinclair Ed. Woodhead Publishing Ltd., Cambridge, England. 2015. Pp 79-95.
Chen, J. Y. and Sun, L. Functional Regenerated Cellulose Fibers. U.S. Patent Application Publication, No.: US 2019/0136415A1, May 9, 2019.
Allen, J. (2007). Photoplethysmography and its application in clinical physiological measurement. Physiological measurement, 28(3), R1.
Allen, J. E., Clunie, G. M., & Winiker, K. (2021). Ultrasound: an emerging modality for the dysphagia assessment toolkit?. Current opinion in otolaryngology & head and neck surgery, 29(3), 213.
Amjadi, M., Pichitpajongkit, A., Lee, S., Ryu, S., & Park, I. (2014). Highly stretchable and sensitive strain sensor based on silver nanowire-elastomer nanocomposite. ACS nano, 8(5), 5154-5163.
Aviv, J. E. (2000). Prospective, Randomized Outcome Study of Endoscopy Versus Modified Barium Swallow in Patients With Dysphagia. The Laryngoscope, 110(4), 563-574. https://doi.org/10.1097/00005537-200004000-00008
Bhattacharyya, N. (2014). The prevalence of dysphagia among adults in the United States. Otolaryngology—Head and Neck Surgery, 151(5), 765-769.
Borowitz, K. C., & Borowitz, S. M. (2018). Feeding problems in infants and children: assessment and etiology. Pediatric clinics, 65(1), 59-72.
Chen, Y., Lu, S., Zhang, S., Li, Y., Qu, Z., Chen, Y., Lu, B., Wang, X., & Feng, X. (2017). Skin-like biosensor system via electrochemical channels for noninvasive blood glucose monitoring. Science advances, 3(12), e1701629.
Chu Jennifer. (2022). Engineers fabricate a chip-free, wireless electronic “skin.” MIT Department of Mechanical Engineering. https://meche.mit.edu/news-media/engineers-fabricate-chip-free-wireless-electronic-% E2%80%9Cskin % E2%80%9D
Collins, F. S., & Varmus, H. (2015). A New Initiative on Precision Medicine. New England Journal of Medicine, 372(9), 793-795. https://doi.org/10.1056/nejmp1500523
Constantinescu, G., Jeong, J.-W., Li, X., Scott, D. K., Jang, K.-I., Chung, H.-J., Rogers, J. A., & Rieger, J. (2016). Epidermal electronics for electromyography: An application to swallowing therapy. Medical Engineering &amp; Physics, 38(8), 807-812. https://doi.org/10.1016/j.medengphy.2016.04.023
Cortes, C., & Vapnik, V. (1995). Support-vector networks. Machine Learning, 20(3), 273-297. https://doi.org/10.1007/bf00994018
Cover, T., & Hart, P. (1967). Nearest neighbor pattern classification. IEEE transactions on information theory, 13(1), 21-27.
Crary, M. A., Mann, G. D. C., & Groher, M. E. (2005). Initial psychometric assessment of a functional oral intake scale for dysphagia in stroke patients. Archives of physical medicine and rehabilitation, 86(8), 1516-1520.
Dudik, J. M., Kurosu, A., Coyle, J. L., & Sejdić, E. (2015). A comparative analysis of DBSCAN, K-means, and quadratic variation algorithms for automatic identification of swallows from swallowing accelerometry signals. Computers in biology and medicine, 59, 10-18.
Ertekin, C., & Aydogdu, I. (2003). Neurophysiology of swallowing. Clinical Neurophysiology, 114(12), 2226-2244.

Esteva, A., Robicquet, A., Ramsundar, B., Kuleshov, V., Depristo, M., Chou, K., Cui, C., Corrado, G., Thrun, S., & Dean, J. (2019). A guide to deep learning in healthcare. Nature Medicine, 25(1), 24-29. https://doi.org/10.1038/s41591-018-0316-z
Ferrari, M., & Quaresima, V. (2012). A brief review on the history of human functional near-infrared spectroscopy (fNIRS) development and fields of application. Neuroimage, 63(2), 921-935.
GlobeNewswire. (2018). [Image in ROHM's new high-speed optical heart rate sensor optimized for measuring blood pressure, stress and vascular age]. GlobeNewswire News Room. https://www.globenewswire.com/fr/news-release/2018/02/21/1372795/0/en/ROHM-s-New-High-Speed-Optical-Heart-Rate-Sensor-Optimized-for-Measuring-Blood-Pressure-Stress-and-Vascular-Age.html
González-Fernández, M., Ottenstein, L., Atanelov, L., & Christian, A. B. (2013). Dysphagia after stroke: an overview. Current Physical Medicine and Rehabilitation Reports, 1(3), 187-196. https://doi.org/10.1007/s40141-013-0017-y
Gulshan, V., Peng, L., Coram, M., Stumpe, M. C., Wu, D., Narayanaswamy, A., Venugopalan, S., Widner, K., Madams, T., Cuadros, J., Kim, R., Raman, R., Nelson, P. C., Mega, J. L., & Webster, D. R. (2016). Development and Validation of a Deep Learning Algorithm for Detection of Diabetic Retinopathy in Retinal Fundus Photographs. JAMA, 316(22), 2402. https://doi.org/10.1001/jama.2016.17216
Hammock, M. L., Chortos, A., Tee, B. C. K., Tok, J. B. H., & Bao, Z. (2013). 25th anniversary article: the evolution of electronic skin (e-skin): a brief history, design considerations, and recent progress. Advanced materials, 25(42), 5997-6038.
Hashimoto, H., Hirata, M., Takahashi, K., Kameda, S., Katsuta, Y., Yoshida, F., Hattori, N., Yanagisawa, T., Palmer, J., Oshino, S., Yoshimine, T., & Kishima, H. (2018). Non-invasive quantification of human swallowing using a simple motion tracking system. Scientific Reports, 8(1). https://doi.org/10.1038/s41598-018-23486-0
Heuschmann, P. U. (2004). Predictors of In-Hospital Mortality and Attributable Risks of Death After Ischemic Stroke<subtitle> The German Stroke Registers Study Group</subtitle>. Archives of Internal Medicine, 164(16), 1761. https://doi.org/10.1001/archinte.164.16.1761
Huang, B., Li, M., Mei, T., McCoul, D., Qin, S., Zhao, Z., & Zhao, J. (2017). Wearable Stretch Sensors for Motion Measurement of the Wrist Joint Based on Dielectric Elastomers. Sensors, 17(12), 2708. https://doi.org/10.3390/s17122708
Jain, A. K., Mao, J., & Mohiuddin, K. M. (1996). Artificial neural networks: A tutorial. Computer, 29(3), 31-44.
Jameson, J. L., & Longo, D. L. (2015). Precision medicine-personalized, problematic, and promising. Obstetrical & gynecological survey, 70(10), 612-614.
Kalantarian, H., Alshurafa, N., Le, T., & Sarrafzadeh, M. (2015). Monitoring eating habits using a piezoelectric sensor-based necklace. Computers in biology and medicine, 58, 46-55.
Kantarcigil, C., Kim, M. K., Chang, T., Craig, B. A., Smith, A., Lee, C. H., & Malandraki, G. A. (2020). Validation of a novel wearable electromyography patch for monitoring submental muscle activity during swallowing: a randomized crossover trial. Journal of Speech, Language, and Hearing Research, 63(10), 3293-3310.
Kim, M. K., Kantarcigil, C., Kim, B., Baruah, R. K., Maity, S., Park, Y., Kim, K., Lee, S., Malandraki, J. B., & Avlani, S. (2019). Flexible submental sensor patch with remote monitoring controls for management of oropharyngeal swallowing disorders. Science advances, 5(12), eaay3210.
Kuramoto, N., Jayatilake, D., Hidaka, K., & Suzuki, K. (2016). Smartphone-based swallowing monitoring and feedback device for mealtime assistance in nursing homes. In 2016 38th Annual International Conference of the IEEE Engineering in Medicine and Biology Society (EMBC) (pp. 5781-5784). IEEE.
Leder, S. B., Acton, L. M., Lisitano, H. L., & Murray, J. T. (2005). Fiberoptic Endoscopic Evaluation of Swallowing (FEES) with and without Blue-Dyed Food. Dysphagia, 20(2), 157-162. https://doi.org/10.1007/s00455-005-0009-x
Lee, J., Sejdić, E., Steele, C. M., & Chau, T. (2010). Effects of liquid stimuli on dual-axis swallowing accelerometry signals in a healthy population. Biomedical engineering online, 9(1), 1-14.
Lee, J., Steele, C., & Chau, T. (2008). Time and time-frequency characterization of dual-axis swallowing accelerometry signals. Physiological measurement, 29(9), 1105.
Lee, Y., Nicholls, B., Sup Lee, D., Chen, Y., Chun, Y., Siang Ang, C., & Yeo, W.-H. (2017). Soft Electronics Enabled Ergonomic Human-Computer Interaction for Swallowing Training. Scientific Reports, 7(1), 46697. https://doi.org/10.1038/srep46697
Leonard, R., & Kendall, K. (2018). Dysphagia assessment and treatment planning: a team approach. Plural publishing.
Li, P., Zhao, L., Jiang, Z., Yu, M., Li, Z., Zhou, X., & Zhao, Y. (2019). A wearable and sensitive graphene-cotton based pressure sensor for human physiological signals monitoring. Scientific Reports, 9(1). https://doi.org/10.1038/s41598-019-50997-1
Li, Y., Miao, X., Chen, J. Y., Jiang, G., & Liu, Q. (2021). Sensing performance of knitted strain sensor on two-dimensional and three-dimensional surfaces. Materials & Design, 197, 109273.
Lin, J., Peng, Z., Liu, Y., Ruiz-Zepeda, F., Ye, R., Samuel, E. L., Yacaman, M. J., Yakobson, B. I., & Tour, J. M. (2014). Laser-induced porous graphene films from commercial polymers. Nature communications, 5(1), 5714.
Litjens, G., Kooi, T., Bejnordi, B. E., Setio, A. A. A., Ciompi, F., Ghafoorian, M., Van Der Laak, J. A., Van Ginneken, B., & Sánchez, C. I. (2017). A survey on deep learning in medical image analysis. Medical image analysis, 42, 60-88.
Logemann, J. (1984). Evaluation and treatment of swallowing disorders. NSSLHA Journal, (12), 38-50.
Logemann, J. A. (1998). The evaluation and treatment of swallowing disorders. Current Opinion in Otolaryngology & Head and Neck Surgery, 6(6), 395-400.
Longval, M., Welch-West, P., Cl, M., Slp, B., Benton, B., Rdh, R., & Teasell. (n.d.). Clinical Guidebook 3. Dysphagia, Aspiration, and Nutrition Post Acquired Brain Injury. https://erabi.ca/wp-content/uploads/2019/06/Clinical-Guidebook_Ch3_Dysphagia-1.pdf
Marik, P. E., & Kaplan, D. (2003). Aspiration pneumonia and dysphagia in the elderly. Chest, 124(1), 328-336.
Martin-Harris, B., Brodsky, M. B., Michel, Y., Castell, D. O., Schleicher, M., Sandidge, J., Maxwell, R., & Blair, J. (2008a). MBS Measurement Tool for Swallow Impairment-MBSImp: Establishing a Standard. Dysphagia, 23(4), 392-405. https://doi.org/10.1007/s00455-008-9185-9
Martin-Harris, B., Brodsky, M. B., Michel, Y., Castell, D. O., Schleicher, M., Sandidge, J., Maxwell, R., & Blair, J. (2008b). MBS measurement tool for swallow impairment-MBSImp: establishing a standard. Dysphagia, 23, 392-405.
Mccullough, G. H., Wertz, R. T., Rosenbek, J. C., Mills, R. H., Ross, K. B., & Ashford, J. R. (2000). Inter- and intrajudge reliability of a clinical examination of swallowing in adults. Dysphagia, 15(2), 58-67.
Medbullets. (2015). Swallowing phase. Medbullets Step 1. https://step1.medbullets.com/gastrointestinal/111036/swallowing
Mohannak, N., Pattison, G., Hird, K., & Needham, M. (2019). Dysphagia-Trouble Swallowing. Myositis Support & Understanding. https://understandingmyositis.org/myositis-complications/dysphagia/
O'Reilly, M., Caulfield, B., Ward, T., Johnston, W., & Doherty, C. (2018). Wearable Inertial Sensor Systems for Lower Limb Exercise Detection and Evaluation: A Systematic Review. Sports Medicine, 48(5), 1221-1246. https://doi.org/10.1007/s40279-018-0878-4
Obermeyer, Z., & Emanuel, E. J. (2016). Predicting the Future-Big Data, Machine Learning, and Clinical Medicine. New England Journal of Medicine, 375(13), 1216-1219. https://doi.org/10.1056/nejmp1606181
Patel, S., Park, H., Bonato, P., Chan, L., & Rodgers, M. (2012). A review of wearable sensors and systems with application in rehabilitation. Journal of NeuroEngineering and Rehabilitation, 9(1), 21. https://doi.org/10.1186/1743-0003-9-21
Perlman, A. L., Palmer, P. M., McCulloch, T. M., Vandaele, D. J. (1999). Electromyographic activity from human laryngeal, pharyngeal, and submental muscles during swallowing. Journal of Applied Physiology, 86(5), 1663-1669.
Quinlan, J. R. (1986). Induction of decision trees. Machine Learning, 1(1), 81-106. https://doi.org/10.1007/bf00116251
Rofes, L., Muriana, D., Palomeras, E., Vilardell, N., Palomera, E., Alvarez-Berdugo, D., Casado, V., & Clavé, P. (2018). Prevalence, risk factors and complications of oropharyngeal dysphagia in stroke patients: A cohort study. Neurogastroenterology &amp; Motility, 30(8), e13338. https://doi.org/10.1111/nmo.13338
Sasegbon, A., & Hamdy, S. (2017). The anatomy and physiology of normal and abnormal swallowing in oropharyngeal dysphagia. Neurogastroenterology & Motility, 29(11), e13100.
Savitzky, A., & Golay, M. J. (1964). Smoothing and differentiation of data by simplified least squares procedures. Analytical chemistry, 36(8), 1627-1639.
Schwirtz, A. (n.d.). Electromyography. Startseite-Fakultät für Sport-und Gesundheitswissenschaften. https://www.sg.tum.de/en/biomechanik/lab/electromyography/
Sejdic, E., Steele, C. M., & Chau, T. (2009). Segmentation of dual-axis swallowing accelerometry signals in healthy subjects with analysis of anthropometric effects on duration of swallowing activities. IEEE Transactions on Biomedical Engineering, 56(4), 1090-1097.
Shieh, W.-Y., Wang, C.-M., & Chang, C.-S. (2015). Development of a Portable Non-Invasive Swallowing and Respiration Assessment Device. Sensors, 15(6), 12428-12453. https://doi.org/10.3390/s150612428
Shull, P. B., & Damian, D. D. (2015). Haptic wearables as sensory replacement, sensory augmentation and trainer—a review. Journal of NeuroEngineering and Rehabilitation, 12(1). https://doi.org/10.1186/s12984-015-0055-z
Smithard, D. G., O′NEILL, P. A., Park, C., England, R., Renwick, D. S., Wyatt, R., Morris, J., Martin, D. F., & Group, N. W. D. (1998). Can bedside assessment reliably exclude aspiration following acute stroke? Age and ageing, 27(2), 99-106.
Sörös, P., Inamoto, Y., & Martin, R. E. (2009). Functional brain imaging of swallowing: An activation likelihood estimation meta-analysis. Human Brain Mapping, 30(8), 2426-2439. https://doi.org/10.1002/hbm.20680
Speech-Language Pathology. (n.d.). Www.uofmhealth.org. Retrieved Jul. 27, 2023, from http://uofmhealth.org/conditions-treatments/rehabilitation/speechlanguage
Steinhubl, S. R., Muse, E. D., & Topol, E. J. (2015). The emerging field of mobile health. Science Translational Medicine, 7(283), 283rv283-283rv283. https://doi.org/10.1126/scitranslmed.aaa3487
Stengel, M. (2017). DIY project-Wearable Imu Tracking Sensor. in magic we trust. https://inmagicwetrust.wordpress.com/2015/11/04/diy-project-wearable-imu-tracking-sensor/
Stepp, C. E. (2012). Surface electromyography for speech and swallowing systems: Measurement, analysis, and interpretation.
Sulzer, J., & Karfeld-Sulzer, L. S. (2021). Our child's TBI: a rehabilitation engineer's personal experience, technological approach, and lessons learned. Journal of NeuroEngineering and Rehabilitation, 18(1). https://doi.org/10.1186/s12984-021-00862-y
Takahashi, K., Groher, M., & Michi, K.-I. (1994). Methodology for detecting swallowing sounds. Dysphagia, 9(1). https://doi.org/10.1007/bf00262760
Tao, L.-Q., Tian, H., Liu, Y., Ju, Z.-Y., Pang, Y., Chen, Y.-Q., Wang, D.-Y., Tian, X.-G., Yan, J.-C., & Deng, N.-Q. (2017). An intelligent artificial throat with sound-sensing ability based on laser induced graphene. Nature communications, 8(1), 14579.
Vaiman, M., Eviatar, E., & Segal, S. (2004). Surface electromyographic studies of swallowing in normal subjects: a review of 440 adults. Report 3. Qualitative data. Otolaryngology-Head and Neck Surgery, 131(6), 977-985.
Wang, J., Lu, C., & Zhang, K. (2020). Textile-based strain sensor for human motion detection. Energy & Environmental Materials, 3(1), 80-100.

Wei, Y., Qiao, Y., Jiang, G., Wang, Y., Wang, F., Li, M., Zhao, Y., Tian, Y., Gou, G., & Tan, S. (2019). A wearable skinlike ultra-sensitive artificial graphene throat. ACS nano, 13(8), 8639-8647.
Wilhelm, M. (2017). Soft sensors might make wearables actually wearable. Wired. https://www.wired.com/story/soft-sensors-might-make-wearables-actually-wearable/
W S Coriolano, M. d. G., R Belo, L., Carneiro, D., G Asano, A., Al Oliveira, P. J., da Silva, D. M., & G Lins, O. (2012). Swallowing in patients with Parkinson's disease: a surface electromyography study. Dysphagia, 27, 550-555.
Yamada, T., Hayamizu, Y., Yamamoto, Y., Yomogida, Y., Izadi-Najafabadi, A., Futaba, D. N., & Hata, K. (2011). A stretchable carbon nanotube strain sensor for human-motion detection. Nature nanotechnology, 6(5), 296-301.
Yang, S., Chen, Y. C., Nicolini, L., Pasupathy, P., Sacks, J., Su, B., Yang, R., Sanchez, D., Chang, Y. F., & Wang, P. (2015). “Cut-and-paste” manufacture of multiparametric epidermal sensor systems. Advanced materials, 27(41), 6423-6430.
Zhang, R., Freund, M., Amft, O., Cheng, J., Zhou, B., Lukowicz, P., Fernando, S., & Chabrecek, P. (2016). A generic sensor fabric for multi-modal swallowing sensing in regular upper-body shirts. Proceedings of the 2016 ACM International Symposium on Wearable Computers,
Zhou, Z.-H. (2012). Ensemble methods: foundations and algorithms. CRC press.
Zoratto, D., Chau, T., & Steele, C. (2010). Hyolaryngeal excursion as the physiological source of swallowing accelerometry signals. Physiological measurement, 31(6), 843.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A wearable device band comprising:
a knitted elastic fabric having a length and a height;
one or more conductive fibers stitched or knitted onto a portion of the wearable device and arranged into at least one knitted strain sensor; and
one or more lead fibers stitched or knitted onto a portion of the wearable device, each connected at a first end to the at least one knitted strain sensor, and at a second end to a connector.

2. The wearable device of claim 1, wherein the at least one knitted strain sensor comprises a first, second and third knitted strain sensor.

3. The wearable device of claim 2, wherein each strain sensor is arranged horizontally along the length of the wearable device.

4. The wearable device of claim 3, wherein each knitted strain sensor has a dimension along the length of the wearable device of between 40 mm and 70 mm, and a dimension along the height of between 5 mm and 20 mm.

5. The wearable device of claim 4, wherein a distance between each knitted strain sensor is between 1 mm and 20 mm.

6. The wearable device of claim 5, wherein each knitted strain sensor is formed in a shape selected from: rectangle, oval, circle, and ellipse.

7. The wearable device of claim 6, wherein the wearable device comprises textured filament nylon yarn interwoven with nylon-covered spandex yarn, the one or more conductive fibers comprises a coated nylon filament yarn, and the one or more lead fibers comprise a coated nylon filament yarn.

8. The wearable device of claim 7, wherein the coating for the conductive fibers and lead fibers is selected from: silver, carbon nanotube, graphene, conductive materials, non-conductive materials, and combinations thereof.

9. The wearable device of claim 8, further comprising adjustable fasteners positioned at each end of the length of the wearable device configured to form the wearable device into a loop.

10. The wearable device of claim 8, wherein the wearable device is a garment or a band.

11-17. (canceled)

18. The wearable device of claim 10, wherein the wearable device comprises a turtle-neck or sweater with a collar, and the at least one knitted strain sensor is stitched or knitted into a portion of the neck of the turtle-neck, or collar of the sweater, wherein the at least one knitted strain sensor is positioned over the laryngeal prominence of the subject.

19. A system for measuring swallowing performance in a subject, comprising:
the wearable device of claim 1;
a first computing device connected to the at least one knitted strain sensor, configured to receive signals from the at least one knitted strain sensor; and
a non-transitory computer-readable medium with instructions stored thereon, which when executed by a processor, perform steps comprising:
collecting data from the at least one knitted strain sensor over a time period;
calculating, from the collected data, the likelihood that a swallow was recorded during the time period; and
calculating a swallow score from the collected data when the likelihood exceeds a predetermined threshold.

20. The system of claim 19, wherein the first computing device is positioned on the wearable device.

21. The system of claim 19, wherein the first computing device further comprises a wireless transceiver and the instructions further comprise collecting data from the at least one knitted strain sensor and transmitting the data via a wireless communication interface to a second computing device.

22. (canceled)

23. A method of measuring swallowing performance in a subject, comprising:
fitting a subject with the wearable device of claim 1;
collecting strain data from the at least one knitted strain sensor over a time period;
calculating a likelihood that a swallow was recorded during the time period; and
calculating a swallow score from the collected data when the likelihood exceeds a predetermined threshold.

24. (canceled)

25. The method of claim 23, wherein the at least one knitted strain sensor comprises a first, second, and third knitted strain sensor, and the method further comprises the step of positioning the first knitted strain sensor above the laryngeal prominence of the subject, the second knitted strain sensor on the laryngeal prominence, and the third knitted strain sensor below the laryngeal prominence.

26. The method of claim 25, wherein the swallow score is calculated using a machine learning algorithm, and wherein the machine learning algorithm comprises the Chi-Squared (Chi2) algorithm.

27. The method of claim 26, further comprising the steps of:
designating at least a portion of the time period as an event; and
comparing the data collected during the portion of the time period to a set of labeled sensor data to identify the event as a swallow, a cough, breathing, a head movement, or speech.

28. The method of claim 27, further comprising transmitting the swallow score and at least a subset of the strain data to a clinician.

29. The method of claim 28, further comprising displaying strain data and swallow score from the subject to a visual display.

30. (canceled)
